(19) United States
(12) Patent Application Publication
(10) Pub. No.: US 2004/0058880 A1
(43) Pub. Date: Mar. 25, 2004

(54) Aminoglycoside antibiotics as novel anti-infective agents

(76) Inventors: Chang-Hsing Liang, San Diego, CA (US); Kenneth Marby, San Diego, CA (US); David Rabuka, San Diego, CA (US); Alex Romero, San Diego, CA (US); Paul W. M. Sgarbi, Richmond (CA); Steven J. Sucheck, San Diego, CA (US); Youe-Kong Shue, Carlsbad, CA (US); Sulan Yao, San Diego, CA (US)

Correspondence Address: CATCALYST LAW GROUP, APC, 4330 LA JOLLA VILLAGE DRIVE SUITE 220, SAN DIEGO, CA, 92122, US

(21) Appl. No.: 10/606,700

(22) Filed: Jun. 26, 2003

Related U.S. Application Data
(60) Provisional application No. 60/393,161, filed on Jul. 1, 2002.

(51) Int. Cl. C07H 5/06; A61K 31/704; A61K 31/35

(52) U.S. Cl. 514/36; 514/42; 514/459; 514/460; 536/18.7; 549/416; 549/419

(57) ABSTRACT

This invention relates to novel aminoglycoside compounds having antibacterial and anti-infective activity and to pharmaceutical compositions, methods of making and methods of treatment employing the same.

### RELATED APPLICATIONS

Benefit of priority under 35 U.S.C. 119(e) is claimed herein to U.S. Provisional application No. 60/393,161, filed Jul. 1, 2002. The disclosure of the above-reference application is incorporated by reference in its entirety herein.

### FIELD OF THE INVENTION

This invention relates to compounds having antibacterial activity and to pharmaceutical compositions, methods of making and methods of treatment employing the same.

### BACKGROUND OF THE INVENTION

The aminoglycosides (AMGs) are a class of cationic carbohydrate-based antibiotics that possess potent antimicrobial activity against gram-positive as well as gram-negative bacteria. They are frequently used in the treatment of opportunistic infections such as Pseudomonas aeruginosa, Acinetobacter species, Streptococci viridans, Proteus species, Hemophilus influenzae, Citrobacter species, Serratia marcescens, Staphylococcus aureus, Streptococcus pyogenes, Escherichia coli, Enterobacter species, Enterococcus faecalis, Enterococcus faecium Klebsiella pneumoniae and Providencia stuartii as well as other bacteria known to cause skin and eye infection, blood infection, urinary tract infection, respiratory infection, infection in burn victims and in cancer and AIDS patients whose immune systems have been compromised. They are also used in the treatment of severe infections of the abdomen, as well as bacteremia and endocarditis. AMGs act by binding to the decoding region (A-site) on the 30s domains in bacterial ribosomes and interfere with the fidelity of protein synthesis (Davies, J.; Gorini, L. and Davis, B. D. Mol. Pharmacol. 1965, 1, 93; Moazed, D. and Noller, H. F. Nature 1987, 327, 389). However, the widespread use of AMG antibiotics over the past 70 years has resulted in the development of a host of antibiotic-resistant pathogens. Numerous examples of tobramycin and gentamicin resistant infections have been documented in both the hospital and out patient settings (Byrne, M. E.; Rouch, D. A.; Skurray, R. A. Gene 1989, 81, 361). The present clinically used AMG antibiotics may be ineffective against these emerging AMGs resistant mutants. Therefore, it is of critical importance to develop and provide new drugs with broad-spectrum activity, particularly against drug-resistant strains.

### SUMMARY OF THE INVENTION

The present invention relates to aminoglycosides and methods for making and using such compounds.

In one aspect of the present invention are new aminoglycosides having the structure of (I), as well as pharmaceutically acceptable salts, prodrugs and solvates thereof:

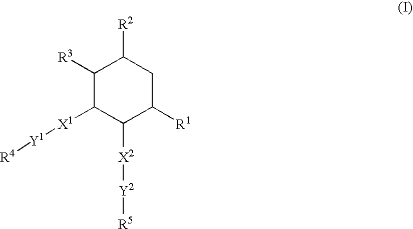

wherein,

R¹ and R² are independently amino, protected amino or modified amino,

X¹ and X² are independently O, S or NH,

Y¹ and Y² are independently a bond or a divalent linking group,

R³ is selected from the group consisting of the formula (II) or (III):

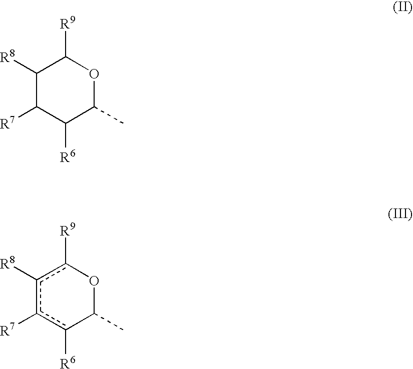

R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1).

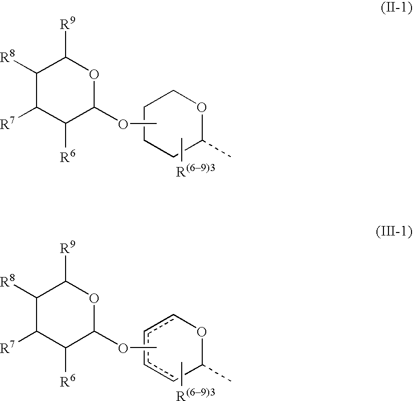

one of R⁴ and R⁵ is hydrogen, hydroxyl protecting or modified hydroxyl group when one of Y¹ or Y² is a bond and the other is selected from a group consisting of formula (II), (III), (IV), (V), (VI) or (VII):

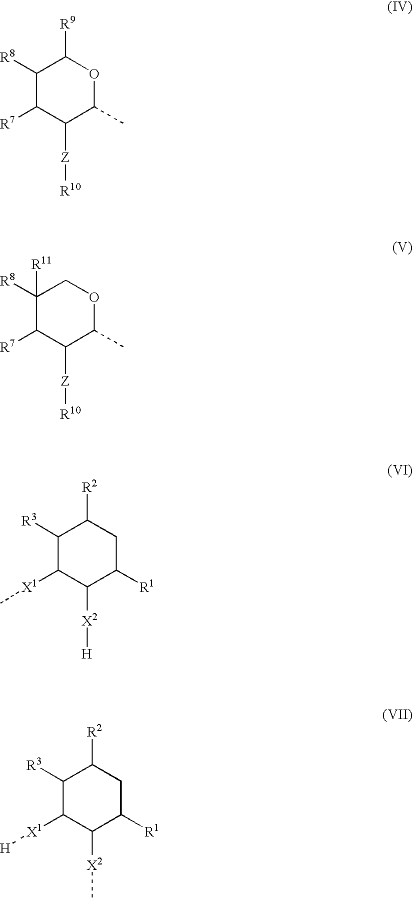

Z can be O, S or NH,

R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen. R⁷, R⁸ and R⁹ can be independently another mono- or disaccharide (II), including disaccharides (IV-1) and (V-1)

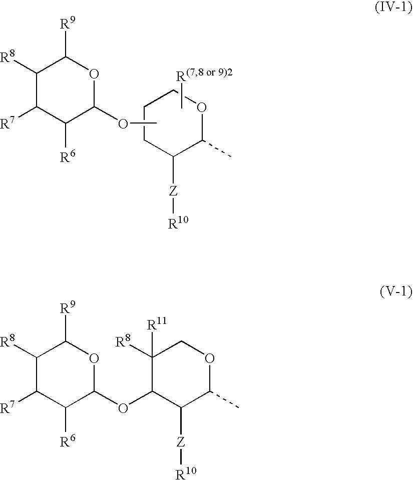

R¹⁰ can be hydrogen, an alkyl group, an amine protecting group, modified amino, hydroxyl protecting or modified hydroxyl group, and

R¹¹ can be a hydrogen, halogen or alkyl group.

In another aspect of the present invention are methods for synthesizing new aminoglycosides having the structure of (1), as well as pharmaceutically acceptable salts, prodrugs and solvates thereof:

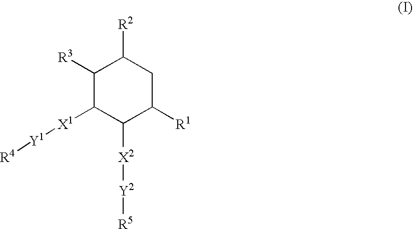

wherein,

R¹ and R² are independently amino, protected amino or modified amino,

X¹ and X² are independently O, S or NH,

Y¹ and Y² are independently a bond or a divalent linking group,

R³ is selected from the group consisting of the formula (II) or (III):

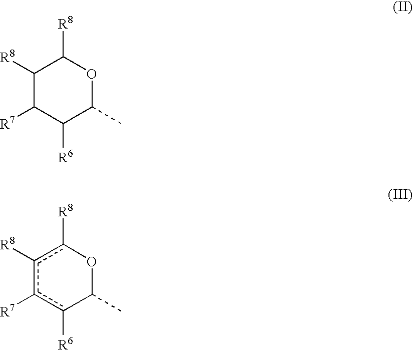

wherein,

R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1).

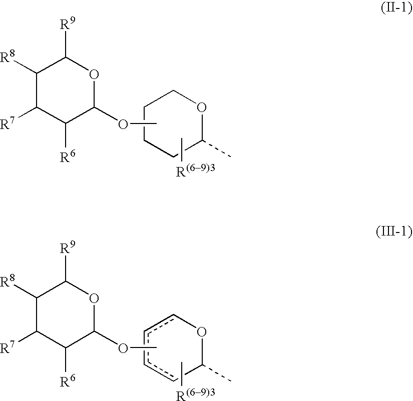

one of R⁴ and R⁵ is hydrogen, hydroxyl protecting or modified hydroxyl group when one of Y¹ or Y² is a bond and the other is selected from a group consisting of formula (II), (III), (IV), (V), (VI) or (VII):

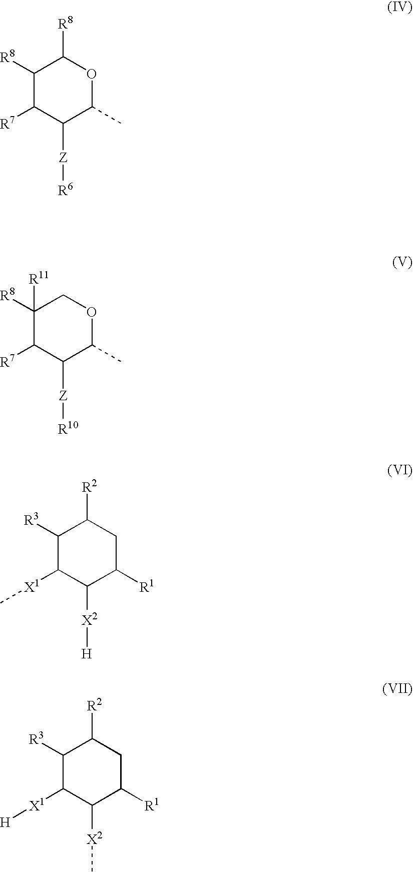

Z can be O, S or NH,

R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1)

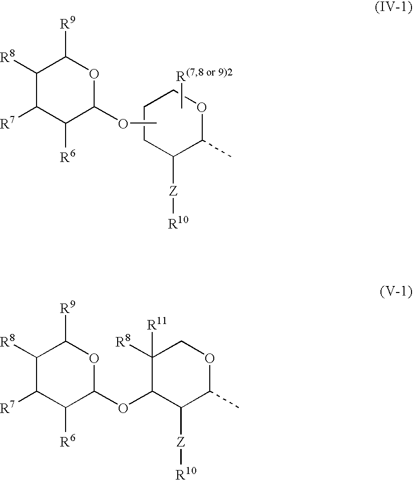

R¹⁰ can be hydrogen, an alkyl group, an amine protecting group, modified amino, hydroxyl protecting or modified hydroxyl group, and

R¹¹ can be a hydrogen, halogen or alkyl group.

In another aspect of the present invention are new intermediates for the synthesis of aminoglycosides having the structure of (Ia), (Ib), (Ic), (Id), (IIa), (IIIa), (IVa) or (Va)

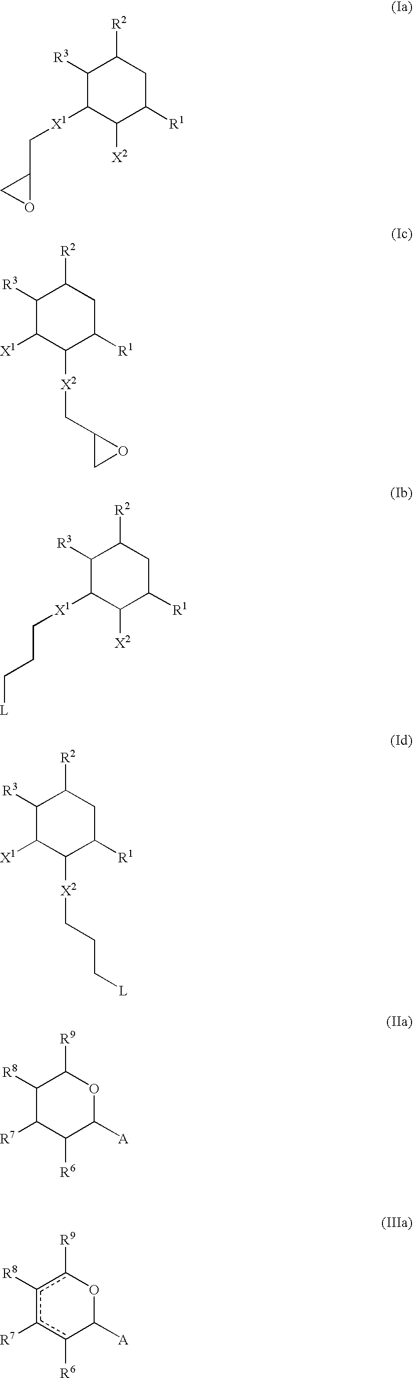

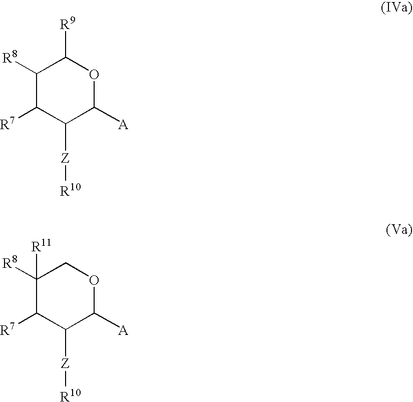

wherein, L is a leaving group,

A is a carbohydrate-activating group,

R¹ and R² are independently amino, protected amino or modified amino,

R³ is selected from the group consisting of the formula (II) or (III),

R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (IIa-1) and (IIIa-1).

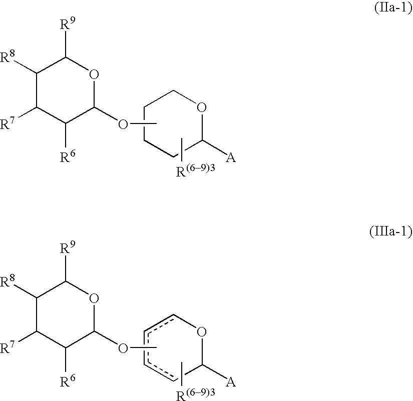

R⁷, R⁸ and R⁹ can also be independently another mono- or disaccharide (II), including disaccharides (IVa-1) and (Va-1)

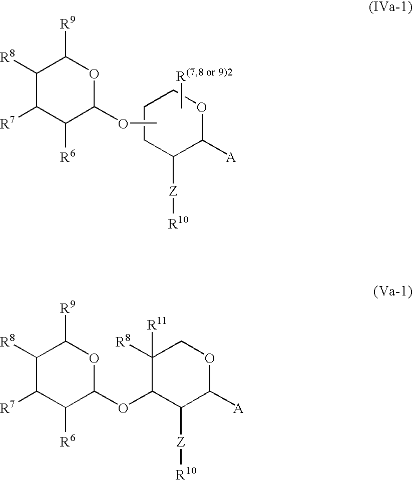

One of X¹ or X² is O, the other can be a protected hydroxyl or modified hydroxyl,

Z can be O, S or NH,

R¹⁰ can be hydrogen, an alkyl group, an amine protecting group, modified amino, hydroxyl protecting or modified hydroxyl group, and

R¹¹ can be a hydrogen, halogen or alkyl group.

In another aspect of the present invention are methods for synthesizing new intermediates for the synthesis of aminoglycosides having the structure of: (Ia), (Ib), (Ic), (Id):

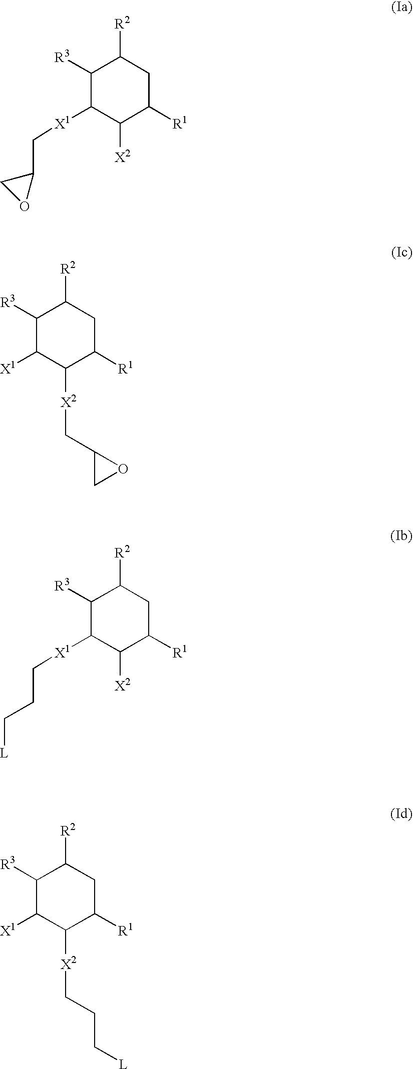

wherein, L is a leaving group,

R¹ and R² are independently amino, protected amino or modified amino,

R³ is selected from the group consisting of the formula (II) or (III), and

One of X¹ or X² is O, the other can be a protected hydroxyl or modified hydroxyl.

In another aspect of the present invention are methods for synthesizing new intermediates, in this case glycosyl donors, for the synthesis of aminoglycosides having the structure of: (IIa), (IIa), (IVa) or (Va)

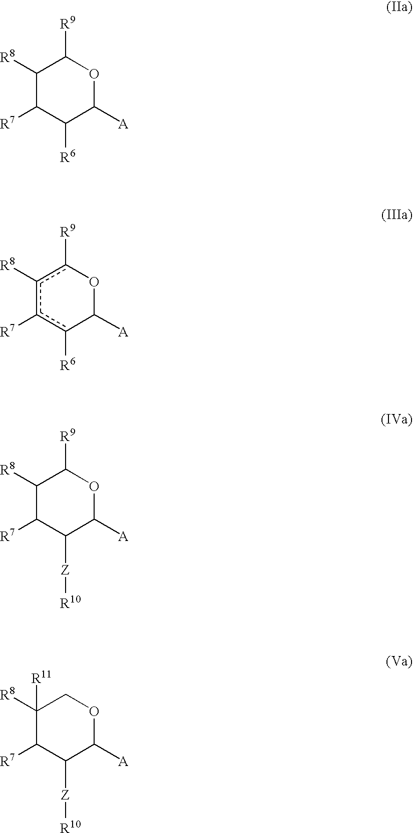

wherein, A is a carbohydrate-activating group,

R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mone- or disaccharide (II), including disaccharides (IIa-1) and (IIIa-1).

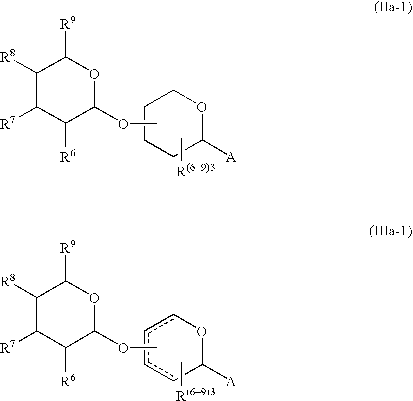

R⁷, R⁸ and R⁹ can also be independently another mono- or disaccharide (II), including disaccharides (IVa-1) and (Va-1)

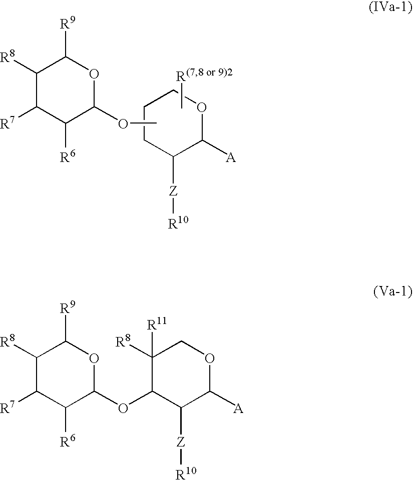

R¹⁰ can be hydrogen, an alkyl group, an amine protecting group, modified amino, hydroxyl protecting or modified hydroxyl group,

R¹¹ can be a hydrogen, halogen or alkyl group, and

Z can be O, S or NH.

Another aspect of the present invention is a pharmaceutical composition for the prophylaxis, amelioration or treatment of a bacterial infection, viral infection, a cancer, or a genetic disorder in mammals avian, fish and reptile species as well as in cell culture, which comprises a therapeutically effective amount of a compound of formula I or a pharmaceutically acceptable salt, prodrug or solvate thereof,

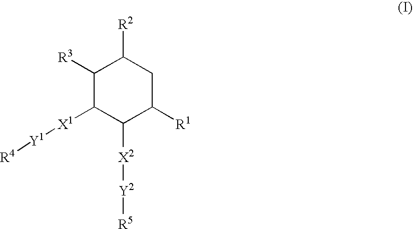

wherein,

R¹ and R² are independently amino, protected amino or modified amino,

X¹ and X² are independently O, S or NH,

Y¹ and Y² are independently a bond or a divalent linking group,

R³ is selected from the group consisting of the formula (II) or (III):

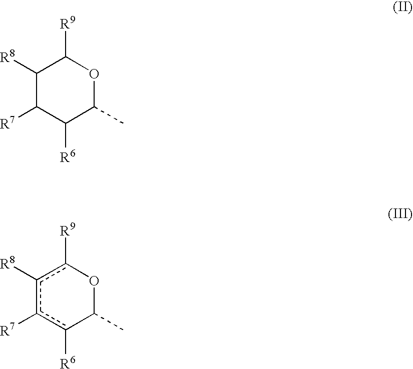

wherein,

R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1).

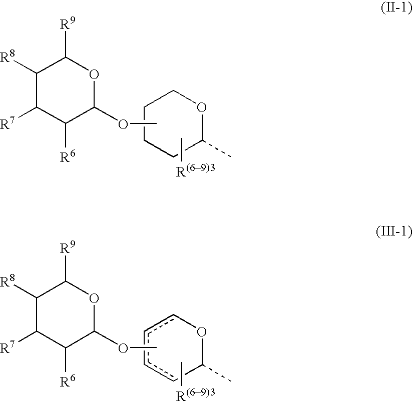

one of R⁴ and R⁵ is hydrogen, hydroxyl protecting or modified hydroxyl group when one of Y¹ or Y² is a bond and the other is selected from a group consisting of formula (II), (III), (IV), (V), (VI) or (VII):

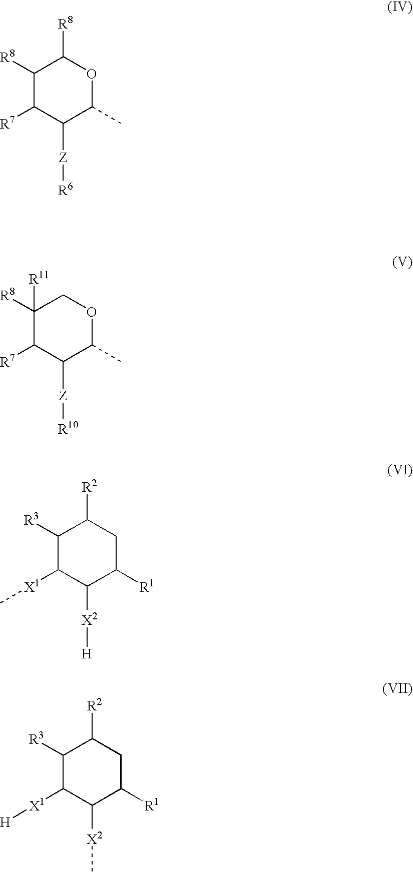

Z can be O, S or NH,

R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1)

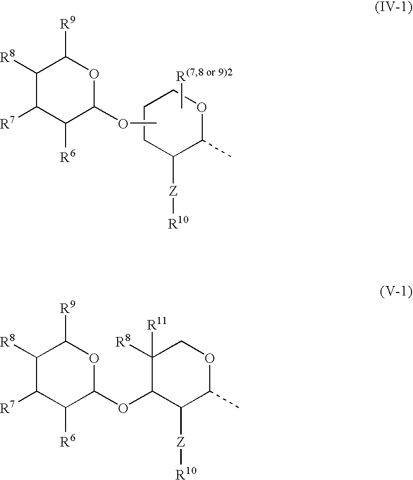

R¹⁰ can be hydrogen, an alkyl group, an amine protecting group, modified amino, hydroxyl protecting or modified hydroxyl group, and

R¹¹ can be a hydrogen, halogen or alkyl group.

Another aspect of the present invention is a method for treating, preventing, or ameliorating a bacterial infection, a viral infection, a cancer, or a genetic disorder in mammals avian, fish and reptile species as well as in cell culture, which comprises administering a therapeutically effective amount of a compound of formula I or a pharmaceutically acceptable salt, prodrug or solvate thereof, and a pharmaceutically acceptable carrier.

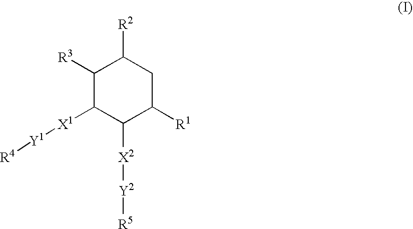

wherein,

R¹ and R² are independently amino, protected amino or modified amino,

X¹ and X² are independently O, S or NH,

Y¹ and Y² are independently a bond or a divalent linking group,

R³ is selected from the group consisting of the formula (II) or (III):

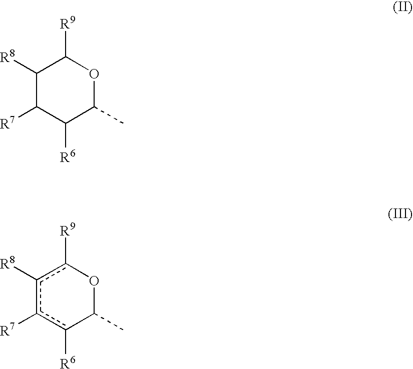

wherein,

R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another monoe- or disaccharide (II), including disaccharides (II-1) and (III-1).

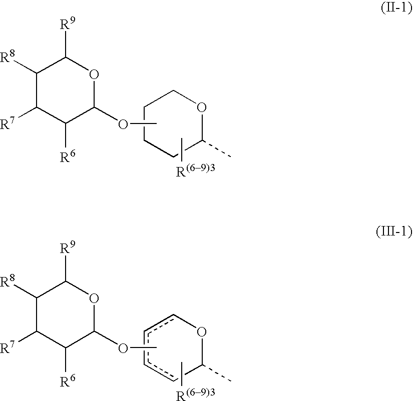

one of R⁴ and R⁵ is hydrogen, hydroxyl protecting or modified hydroxyl 5 group when one of Y¹ or Y² is a bond and the other is selected from a group consisting of formula (II), (III), (IV), (V), (VI) or (VII):

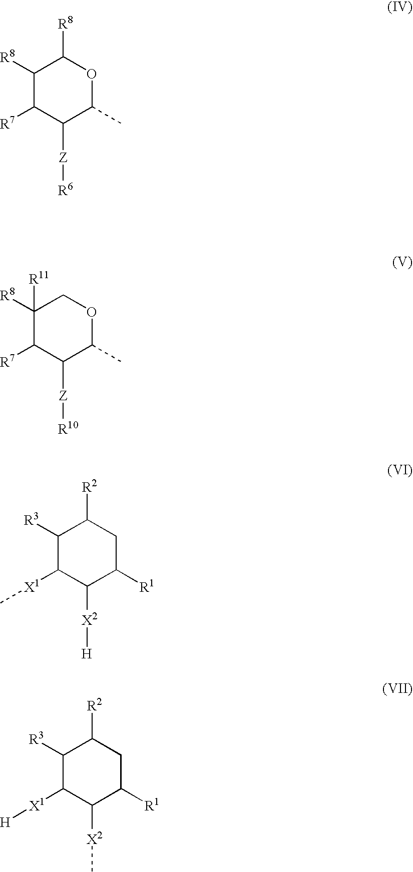

Z can be O, S or NH,

R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1)

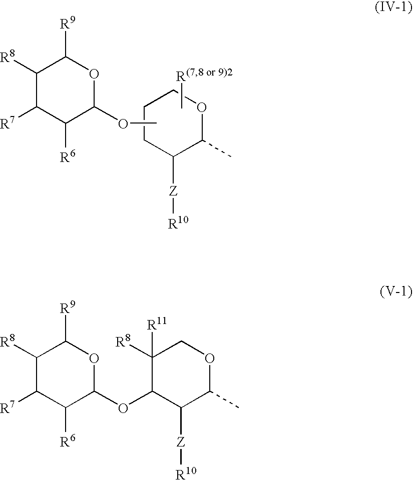

R¹⁰ can be hydrogen, an alkyl group, an amine protecting group, modified amino, hydroxyl protecting or modified hydroxyl group, and

R¹¹ can be a hydrogen, halogen or alkyl group.

Another aspect of the present in invention is an antibacterial, antiviral or antifungal agent comprising a compound of formula 1, and a pharmaceutically acceptable carrier.

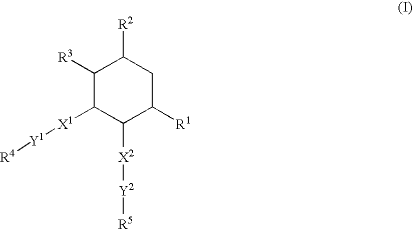

wherein,

R¹ and R² are independently amino, protected amino or modified amino,

X¹ and X² are independently O, S or NH,

Y¹ and Y² are independently a bond or a divalent linking group,

R³ is selected from the group consisting of the formula (II) or (III):

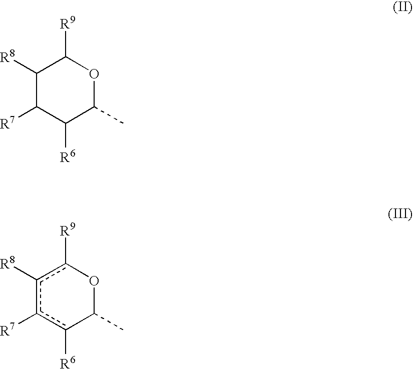

R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1).

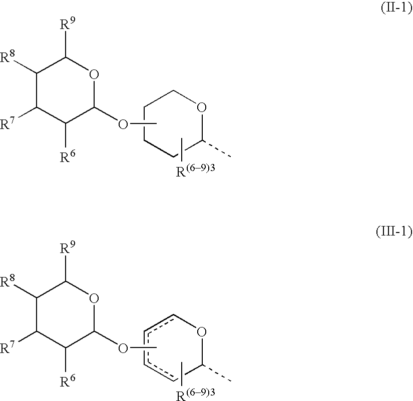

one of R⁴ and R⁵ is hydrogen, hydroxyl protecting or modified hydroxyl group when one of Y¹ or Y² is a bond and the other is selected from a group consisting of formula (II), (III), (IV), (V) or (VI):

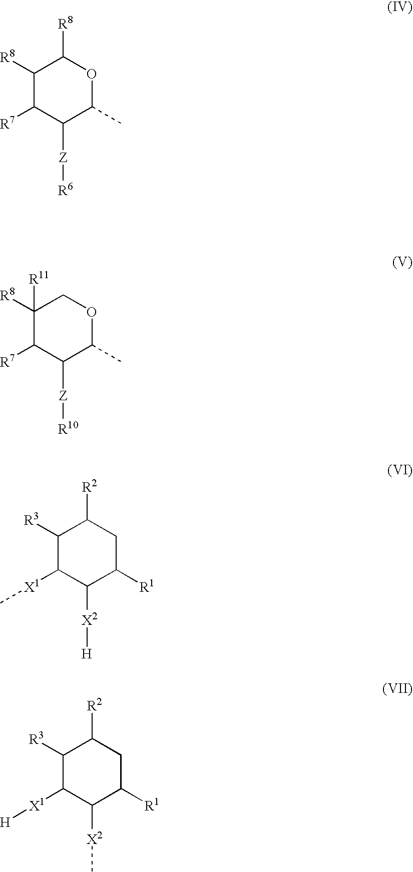

Z can be O, S or NH,

R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1)

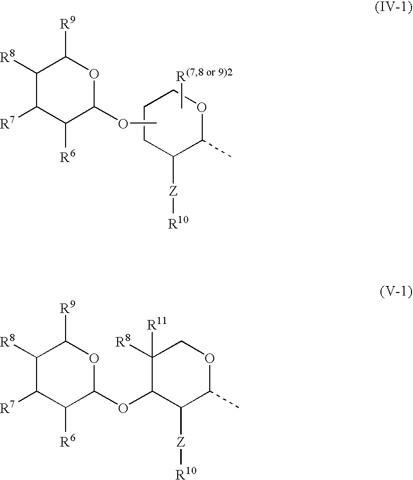

R¹⁰ can be hydrogen, an alkyl group, an amine protecting group, modified amino, hydroxyl protecting or modified hydroxyl group, and

R¹¹ can be a hydrogen, halogen or alkyl group.

Another aspect of the present invention is a method for preventing, inhibiting, or stopping the growth of bacteria on a surface or within the material of the surface, comprising applying to a surface or within the material of the surface an effective amount of an antibacterial agent comprising a compound of formula 1, and an acceptable carrier

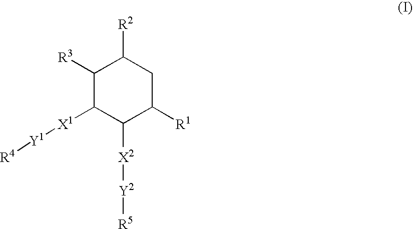

wherein,

R¹ and R² are independently amino, protected amino or modified amino,

X¹ and X² are independently O, S or NH,

Y¹ and Y² are independently a bond or a divalent linking group,

R³ is selected from the group consisting of the formula (II) or (III):

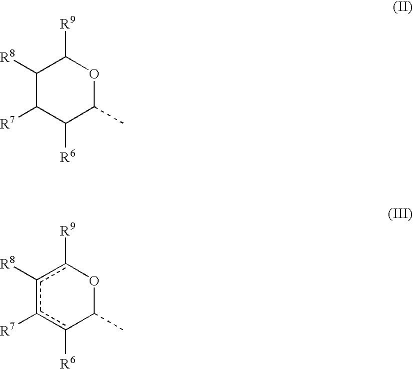

R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1).

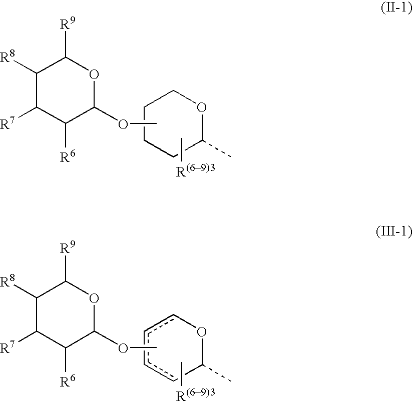

one of R⁴ and R⁵ is hydrogen, hydroxyl protecting or modified hydroxyl 5 group when one of Y¹ or Y² is a bond and the other is selected from a group consisting of formula (II), (III), (IV), (V), (VI) or (VII):

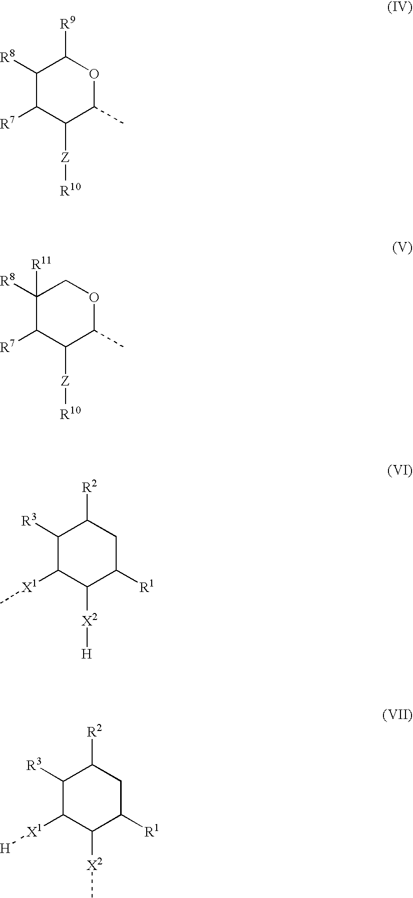

Z can be O, S or NH,

R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1)

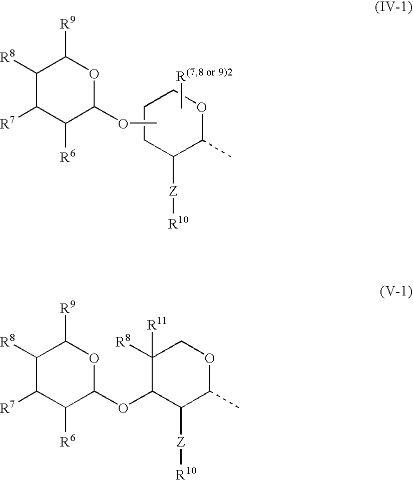

R¹⁰ can be hydrogen, an alkyl group, an amine protecting group, modified amino, hydroxyl protecting or modified hydroxyl group, and

R¹¹ can be a hydrogen, halogen or alkyl group.

### BRIEF DESCRIPTION OF THE FIGURES

FIG. 1 is an example of a 2-desoxystreptamine (2-DOS) ring system.

FIG. 2 is an example of a 2-desoxystreptamine (2-DOS) ring system.

FIG. 3 is an example of a 2-desoxystreptamine (2-DOS) ring system.

FIG. 4 is an example of glycosyl or disaccharide moieties of the present invention substituted at the 4-position of the 2-DOS ring of a 2-desoxystreptamine (2-DOS) ring system.

FIG. 5 is an example of aminoglycoside compounds having the noted 4, 6-substitued ring system.

FIG. 6 is an example of aminogylcoside compounds having glycosyl or disaccharide moieties according to the present invention substituted at the 4-position of the 2-DOS ring and the 6-position of the 2-DOS ring respectively.

FIG. 7 is an example of aminoglycoside compounds having the noted 4, 5-substitued ring system.

FIG. 8 is an example of aminogylcoside compounds having glycosyl or disaccharide moieties according to the present invention substituted at the 4-position of the 2-DOS ring and the 5-position of the 2-DOS ring respectively.

FIG. 9 is an example of aminogylcoside compounds having glycosyl moieties according to the present invention substituted at the 4-position of the 2-DOS ring and a linking group connected to a glycosyl group at the 5- or 6-position of the 2-DOS ring respectively.

FIG. 10 is an example of aminogylcoside compounds having glycosyl moieties according to the present invention substituted at the 4-position of the 2-DOS ring and a linking group connected to a 4-position, 2-DOS glycosylated group at the 5- or 6-position of the 2-DOS ring respectively.

FIG. 11 is an example of fully protected saccharide moieties according to the present invention.

FIG. 12 is an example of saccharide moieties according to the present invention having one free hydroxyl therein.

FIG. 13 is an example of fully protected disaccharide moieties according to the present invention.

### DETAILED DESCRIPTION OF THE INVENTION

All patents, publications and patent applications referred to herein are hereby incorporated by reference in their entireties. The compounds of the present invention may be prepared according to the Schemes below. Unless otherwise indicated, the substituents of the compounds in the schemes are defined as described above.

Definitions

The compounds of the invention comprise asymmetrically substituted carbon atoms. Such asymmetrically substituted carbon atoms can result in the compounds of the invention comprising mixtures of stereoisomers at a particular asymmetrically substituted carbon atom or a single stereoisomer. As a result, racemic mixtures, mixtures of diastereomers, as well as single diastereomers of the compounds of the invention are included in the present invention. The terms “S” and “R” configuration, as used herein, are as defined by the IUPAC 1974 Recommendations for Section E, Fundamental Stereochemistry, Pure Appl. Chem. (1976) 45, 13-30.

The compositions containing the compound(s) of the invention can be administered for prophylactic and/or therapeutic treatments. In therapeutic applications, the compositions are administered to a patient already suffering from an infection, as described above, in an amount sufficient to cure or at least partially arrest the symptoms of the infection. An amount adequate to accomplish this is defined as “therapeutically effective amount or dose.” Amounts effective for this use will depend on the severity and course of the infection, previous therapy, the patient's health status and response to the drugs, and the judgment of the treating physician. In prophylactic applications, compositions containing the compounds of the invention are administered to a patient susceptible to or otherwise at risk of a particular infection. Such an amount is defined to be a “prophylactically effective amount or dose.” In this use, the precise amounts again depend on the patient's state of health, weight, and the like.

Once improvement of the patient's conditions has occurred, a maintenance dose is administered if necessary. Subsequently, the dosage or the frequency of administration, or both, can be reduced, as a function of the symptoms, to a level at which the improved condition is retained. When the symptoms have been alleviated to the desired level, treatment can cease. Patients can, however, require intermittent treatment on a long-term basis upon any recurrence of the disease symptoms.

In general, a suitable effective dose of the compound of the invention will be in the range of 0.1 to 1000 milligrams (mg) per recipient per day, preferably in the range of 1 to 100 mg per day. The desired dosage is preferably presented in one, two, three, four or more subdoses administered at appropriate intervals throughout the day. These subdoses can be administered as unit dosage forms, for example, containing 5 to 1000 mg, preferably 10 to 100 mg of active ingredient per unit dosage form. Preferably, the compounds of the invention will be administered in amounts of between about 1.0 mg/kg to 250 mg/kg of patient body weight, between about one to four times per day.

The term “carbon chain” means a plurality of carbon atoms covalently bonded to one another. The chain may be alkyl, alkenyl, alkynyl, aromatic, conjugated, branched, unbranched, substituted, cyclic, or combinations thereof, etc. The carbon chain may also contain one or more heteroatoms, i.e., atoms other than carbon.

A “pharmacological composition” refers to a mixture of one or more of the compounds described herein, or physiologically acceptable salts thereof, with other chemical components, such as physiologically acceptable carriers and/or excipients. The purpose of a pharmacological composition is to facilitate administration of a compound to an organism.

“Pharmaceutically acceptable salts” of the compounds of the invention include those derived from pharmaceutically acceptable inorganic and organic acids and bases. Examples of suitable acids include hydrochloric, hydrobromic, sulfuric, nitric, perchloric, fumaric, maleic, phosphoric, glycolic, gluconic, lactic, salicylic, succinic, toluene-p-sulfonic, tartaric, acetic, citric, methanesulfonic, formic, benzoic, malonic, naphthalene-2-sulfonic, benzenesulfonic, 1,2 ethanesulfonic acid (edisylate), galactosyl-D-gluconic acid, and the like. Other acids, such as oxalic acid, while not themselves pharmaceutically acceptable, may be employed in the preparation of salts useful as intermediates in obtaining the compounds of this invention and their pharmaceutically acceptable acid addition salts. Salts derived from appropriate bases include alkali metal (e.g., sodium), alkaline earth metal (e.g., magnesium), ammonium and N—(C.sub.1-C.sub.4 alkyl).sub.4.sup.+salts, and the like. Illustrative examples of some of these include sodium hydroxide, potassium hydroxide, choline hydroxide, sodium carbonate, and the like.

A “physiologically acceptable carrier” refers to a carrier or diluent that does not cause significant irritation to an organism and does not abrogate the biological activity and properties of the administered compound.

An “excipient” refers to an inert substance added to a pharmacological composition to further facilitate administration of a compound. Examples of excipients include but are not limited to, calcium carbonate, calcium phosphate, various sugars and types of starch, cellulose derivatives, gelatin, vegetable oils and polyethylene glycols.

The term “alkyl,” alone or in combination, refers to an optionally substituted straight-chain, optionally substituted branched-chain, or optionally substituted cyclic alkyl radical having from 1 to about 30 carbons, preferably 1 to 12 carbons. Examples of alkyl radicals include methyl, ethyl, n-propyl, isopropyl, n-butyl, isobutyl, sec-butyl, tert-butyl, tert-amyl, pentyl, hexyl, heptyl, octyl and the like.

The term “cycloalkyl” embraces cyclic configurations, is subsumed within the definition of alkyl and specifically refers to a monocyclic, bicyclic, tricyclic, and higher multicyclic alkyl radicals wherein each cyclic moiety has from 3 to about 8 carbon atoms. Examples of cycloalkyl radicals include cyclopropyl, cyclobutyl, cyclopentyl, cyclohexyl and the like. A “lower alkyl” is a shorter alkyl, e.g., one containing from 1 to about 6 carbon atoms.

The term “alkenyl,” alone or in combination, refers to an optionally substituted straight-chain, optionally substituted branched-chain, or optionally substituted cyclic alkenyl hydrocarbon radical having one or more carbon-carbon double-bonds and having from 2 to about 30 carbon atoms, more preferably 2 to about 18 carbons. Examples of alkenyl radicals include ethenyl, propenyl, butenyl, 1,4-butadienyl and the like. The term can also embrace cyclic alkenyl structures. A “lower akenyl” refers to an alkenyl having from 2 to about 6 carbons.

The term “alkynyl,” alone or in combination, refers to an optionally substituted straight-chain, optionally substituted branched-chain, or cyclic alkynyl hydrocarbon radical having one or more carbon-carbon triple-bonds and having from 2 to about 30 carbon atoms, more preferably 2 to about 12 carbon atoms. The term also includes optionally substituted straight-chain or optionally substituted branched-chain hydrocarbon radicals having one or more carbon—carbon triple bonds and having from 2 to about 6 carbon atoms as well as those having from 2 to about 4 carbon atoms. Examples of alkynyl radicals include ethynyl, propynyl, butynyl and the like.

The terms heteroalkyl, heteroalkenyl and heteroalkynyl include optionally substituted alkyl, alkenyl and alkynyl structures, as described above, and which have one or more skeletal chain atoms selected from an atom other that carbon, e.g., oxygen, nitrogen, sulfur, phosphorous or combinations thereof.

The term “carbon chain” may embrace any alkyl, alkenyl, alkynyl, or heteroalkyl, heteroalkenyl, or heteroalkynyl group, and may be linear, cyclic, or any combination thereof. If part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining chain length. If the chain contains heteroatoms as part of the backbone, those atoms are not calculated as part of the carbon chain length.

The term “alkoxy,” alone or in combination, refers to an alkyl ether radical, alkyl-O—, wherein the term alkyl is defined as above. Examples of alkoxy radicals include methoxy, ethoxy, n-propoxy, isopropoxy, n-butoxy, iso-butoxy, sec-butoxy, tert-butoxy and the like.

The term “aryloxy,” alone or in combination, refers to an aryl ether radical wherein the term aryl is defined as below. Examples of aryloxy radicals include phenoxy, benzyloxy and the like.

The term “alkylthio,” alone or in combination, refers to an alkyl thio radical, alkyl-S—, wherein the term alkyl is defined as above.

The term “arylthio,” alone or in combination, refers to an aryl thio radical, aryl-S—, wherein the term aryl is defined as below.

The term “aryl,” alone or in combination, refers to an optionally substituted aromatic ring system. The term aryl includes monocyclic aromatic rings, polyaromatic rings and polycyclic aromatic ring systems containing from six to about twenty carbon atoms. The term aryl also includes monocyclic aromatic rings, polyaromatic rings and polycyclic ring systems containing from 6 to about 12 carbon atoms, as well as those containing from 6 to about 10 carbon atoms. The polyaromatic and polycyclic aromatic rings systems may contain from two to four rings. Examples of aryl groups include, without limitation, phenyl, biphenyl, naphthyl and anthryl ring systems.

The term “heteroaryl” refers to optionally substituted aromatic ring systems containing from about five to about 20 skeletal ring atoms and having one or more heteroatoms such as, for example, oxygen, nitrogen, sulfur, and phosphorus. The term heteroaryl also includes optionally substituted aromatic ring systems having from 5 to about 12 skeletal ring atoms, as well as those having from 5 to about 10 skeletal ring atoms. The term heteroaryl may include five- or six-membered heterocyclic rings, polycyclic heteroaromatic ring systems and polyheteroaromatic ring systems where the ring system has two, three or four rings. The terms heterocyclic, polycyclic heteroaromatic and polyheteroaromatic include ring systems containing optionally substituted heteroaromatic rings having more than one heteroatom as described above (e.g., a six membered ring with two nitrogens), including polyheterocyclic ring systems of from two to four rings. The term heteroaryl includes ring systems such as, for example, furanyl, benzofuranyl, chromenyl, pyridyl, pyrrolyl, indolyl, quinolinyl, N-alkyl pyrrolyl, pyridyl-N-oxide, pyrimidoyl, pyrazinyl, imidazolyl, pyrazolyl, oxazolyl, benzothiophenyl, purinyl, indolizinyl, thienyl and the like.

The term “heteroarylalkyl” refers to a C₁-C₄ alkyl group containing a heteroaryl group, each of which may be optionally substituted.

The term “heteroarylthio” refers to the group —S-heteroaryl.

The term “acyloxy” refers to the ester group —OC(O)—R, where R is H, alkyl, alkenyl, alkynyl, aryl, or arylalkyl, wherein the alkyl, alkenyl, alkynyl and arylalkyl groups may be optionally substituted.

The term “carboxy esters” refers to —C(O)OR where R is alkyl, aryl or arylalkyl, wherein the alkyl, aryl and arylalkyl groups may be optionally substituted.

The term “carboxamido” refers to

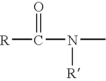

wherein each of R and R′ are independently selected from the group consisting of H, alkyl, aryl and arylalkyl, wherein the alkyl, aryl and arylalkyl groups may be optionally substituted.

The term “arylalkyl,” alone or in combination, refers to an alkyl radical as defined above in which one H atom is replaced by an aryl radical as defined above, such as, for example, benzyl, 2-phenylethyl and the like.

The terms haloalkyl, haloalkenyl, haloalkynyl and haloalkoxy include alkyl, alkenyl, alkynyl and alkoxy structures, as described above, that are substituted with one or more fluorines, chlorines, bromines or iodines, or with combinations thereof.

The terms cycloalkyl, aryl, arylalkyl, heteroaryl, alkyl, alkynyl, alkenyl, haloalkyl and heteroalkyl include optionally substituted cycloalkyl, aryl, arylalkyl, heteroaryl, alkyl, alkynyl, alkenyl, haloalkyl and heteroalkyl groups.

The term “carbocycle” includes optionally substituted, saturated or unsaturated, three- to eight-membered cyclic structures in which all of the skeletal atoms are carbon.

The term “membered ring” can embrace any cyclic structure, including carbocycles and heterocycles as described above. The term “membered” is meant to denote the number of skeletal atoms that constitute the ring. Thus, for example, pyridine, pyran, and thiophan are 6-membered rings and pyrrole, furan, and thiophen are 5-membered rings.

The term “acyl” includes alkyl, aryl, heteroaryl, arylalkyl or heteroarylalkyl substituents attached to a compound via a carbonyl functionality (e.g., —CO-alkyl, —CO-aryl, —CO-arylalkyl or —CO-heteroarylalkyl, etc.).

The term “alkylacylamino” as used herein refers to an alkyl radical appended to an acylamino group.

The term “acylamino” as used herein refers to an acyl radical appended to an amino group.

The term “substituted heterocycle” or heterocyclic group” as used herein refers to any 3-, or 4-membered ring containing a heteroatom selected from nitrogen, oxygen, phosphorus and sulfur or a 5- or 6-membered ring containing from one to three heteroatoms selected from the group consisting of nitrogen, oxygen, phosphorus and sulfur; wherein the 5-membered ring has 0-2 double bounds and the 6-membered ring has 0-3 double bounds; wherein the nitrogen and sulfur atom maybe optionally oxidized; wherein the nitrogen and sulfur heteroatoms maybe optionally quarternized; and including any bicyclic group in which any of the above heterocyclic rings is fused to a benzene ring or another 5- or 6-membered heterocyclic ring independently defined above. Heterocyclics can be unsubstituted or monosubstituted or disubstituted with substituents independly seleceted from hydroxy, halo, oxo (C═O), alkylimino (R—N=wherein R is a alkyl group), amino, alkylamino, dialkylamino, acylaminoalkyl, alkoxy, thioalkoxy, polyalkoxy, alkyl, cycloalkyl or haloalkyl. Examples of heterocyclics include: imidazolyl, pyridyl, piperazinyl, azetidinyl, thiazolyl and triazoly.

The term “divalent linking group” as used herein refers to but are not limited to branched or straight chain groups which can be used to tether two pharmacophores, the following are examples of such groups:

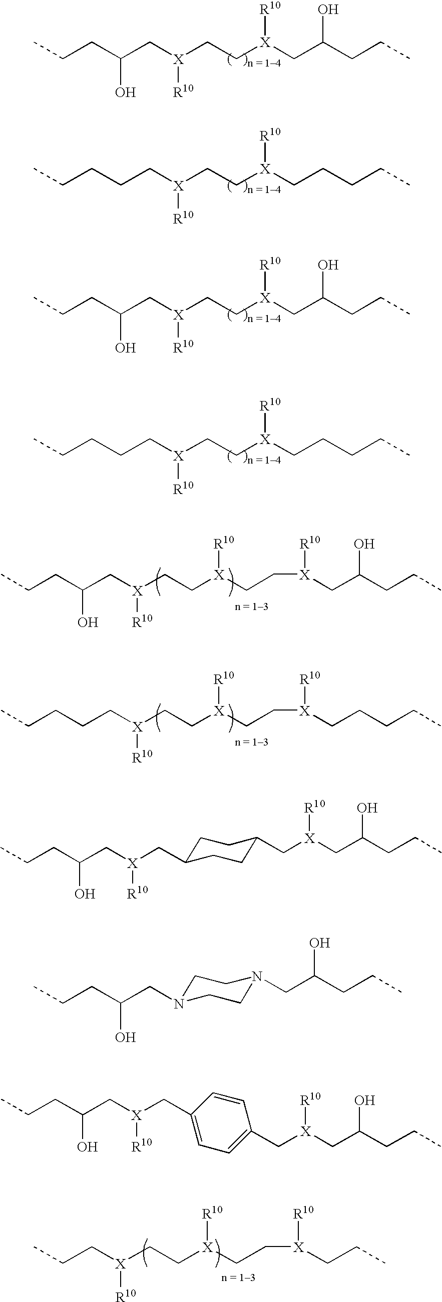

X can be independently O, S or N.

When X=N then R¹⁰ can be hydrogen, an alkyl group, an amine protecting group.

When X is not ═N then R¹⁰ can be a lone pair of electrons.

The term “glycosyl” as used herein refers to any pyranose or furanose saccharide group, including but not limited to D- or L-glucosyl, D- or L-2-deoxy-2-amino-glucosyl and D- or L-3-deoxy-3-amino-glucosyl, etc.

The term “disaccharide” as used herein refers to any pyranose or furanose saccharide group, including but not limited to D- or L-glucosyl, D- or L-2-deoxy-2-amino-glucosyl and D- or L-3-deoxy-3-amino-glucosyl, etc. liked through a glycosidic bond to any other another pyranose or furanose saccharide.

The term “glycosyl donor” as used herein refers to any pyranose or furanose saccharide or disaccharide group capable of glycosylating an acceptor such as hydroxyl, donors include but are not limited to suitably protected thiotoluyl D- or L-glucopyranoside, thiotoluyl D- or L-2-deoxy-2-amino-glucopyranoside and thiotoluyl D- or L-3-deoxy-3-amino-glucopyranoside, etc. The glycosidic linkages can be alpha, beta or alpha/beta mixtures. The following are examples of such saccharide and disaccharide groups:

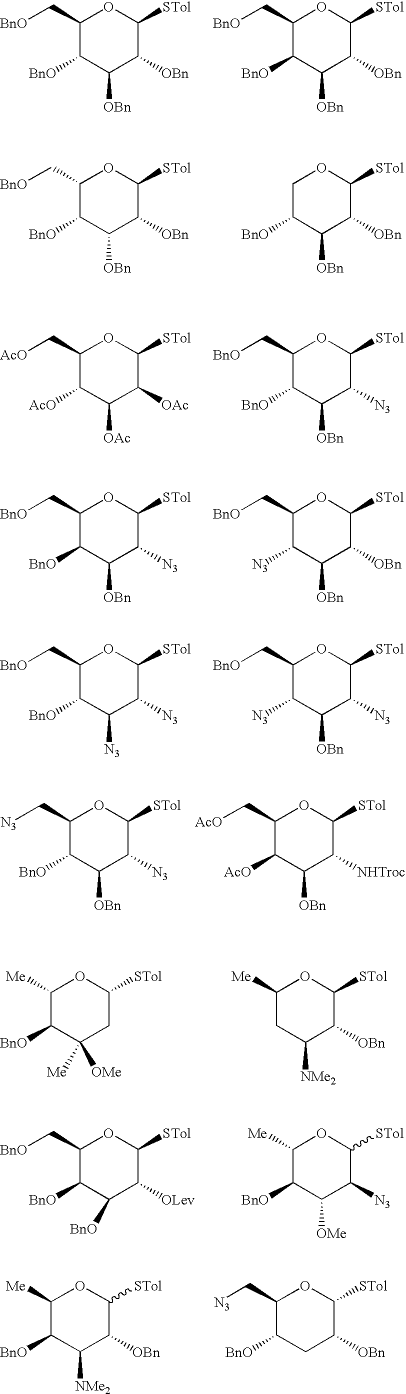

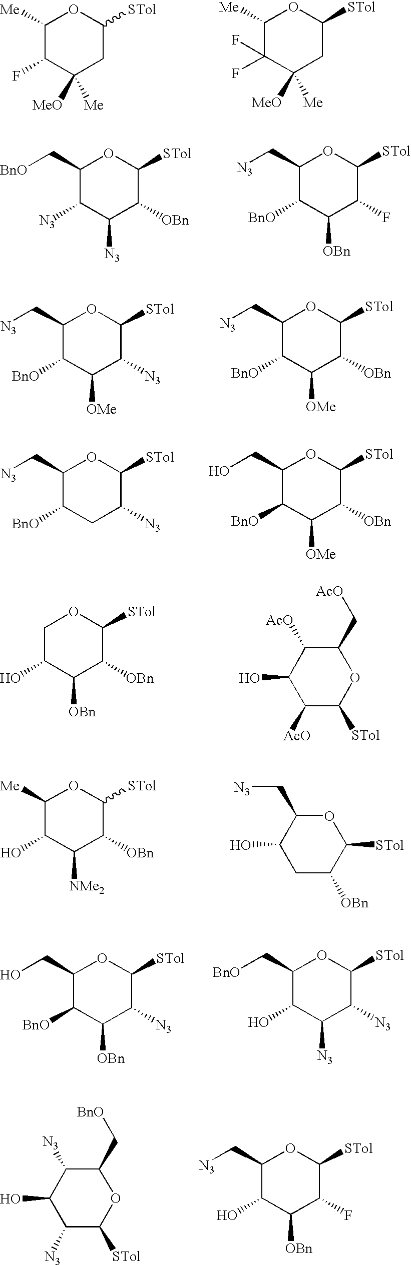

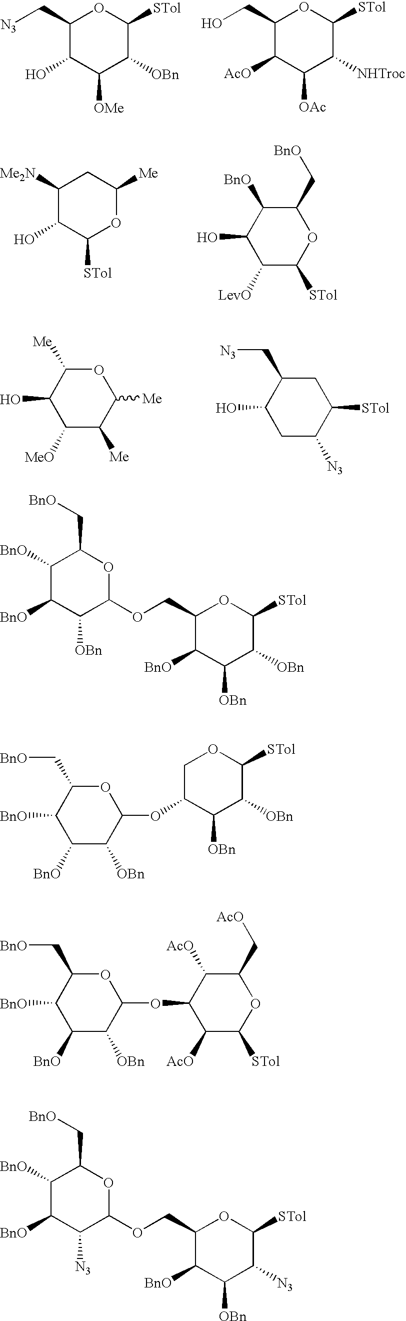

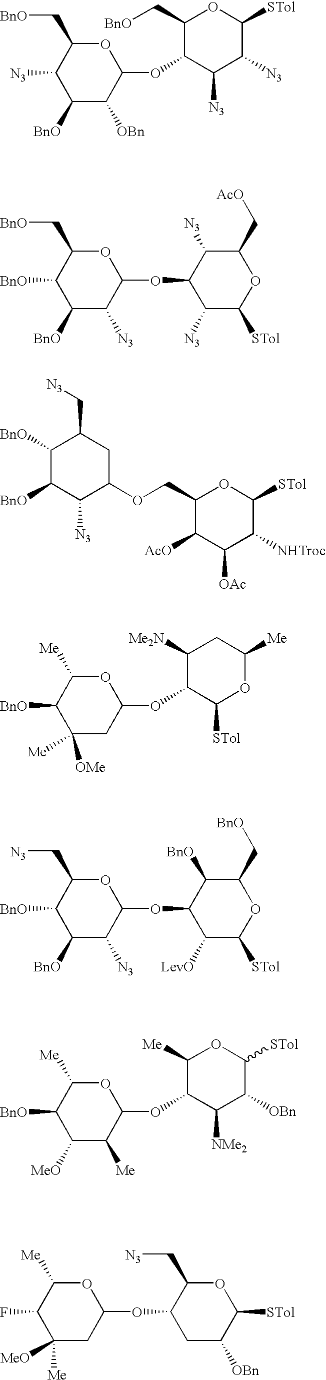

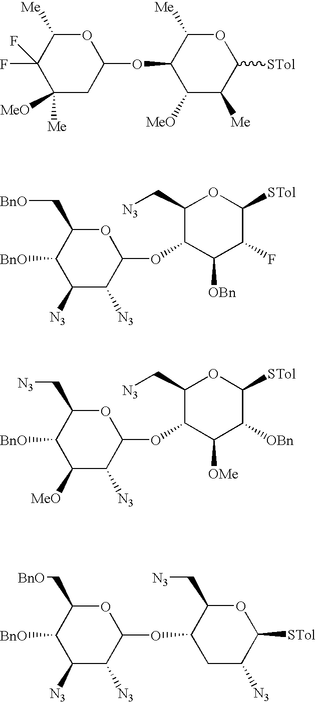

The term “carbohydrate-activating group” as used herein refers to classes of functional groups that when attached to carbohydrates convert then into glycosyl donors. The carbohydrate-activating group is generally located at the anomeric position of the carbohydrate. Activating groups based on the type of anomeric functional group and their activating methods include but are not limited to: glycosyl halides, thioglycosides, 1-O-Acyl sugars, 1-O- and S-carbonates, trichloroimidates, etc.

“Optionally substituted” groups may be substituted or unsubstituted. The substituents of an “optionally substituted” group may include, without limitation, one or more substituents independently selected from the following groups or designated subsets thereof: alkyl, alkenyl, alkynyl, heteroalkyl, haloalkyl, haloalkenyl, haloalkynyl, cycloalkyl, aryl, heteroaryl, arylalkyl, heteroarylalkyl, alkoxy, aryloxy, haloalkoxy, amino, alkylamino, dialkylamino, alkylthio, arylthio, heteroarylthio, oxo, carboxyesters, carboxamido, acyloxy, H, F, Cl, Br, I, CN, NO₂, NH₂, N₃, NHCH₃, N(CH₃)₂, SH, SCH₃, OH, OCH₃, OCF₃, CH₃, CF₃, C(O)CH₃, CO₂CH₃, CO₂H, C(O)NH₂, pyridinyl, thiophene, furanyl, indole, indazol, esters, amides, phosphonates, phosphates, phosphoramides, sulfonates, sulfates, sulphonamides, carbamates, ureas, thioureas, thioamides, thioalkyls. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstututed (e.g., —CH₂CF₃).

The term “halogen” includes F, Cl, Br and I.

The term “protected amino”, “amine protecting group” and “protected aminomethyl” as used herein refers to known amine protecting groups used in the synthetic organic chemistry art and include but are not limited to t-butoxycarbonyl (BOC), benzyloxycarbonyl (Cbz), azide (N₃), 2-trimethylsilylethoxy-carbonyl (Teoc), allyloxycarbonyl (Alloc), 9-fluorenylmethyloxycarbonyl (Fmoc), acyl groups, such as formyl, acetyl, trihaloacetyl, benzoyl, and nitrophenylacetyl, sulfonamide groups, imine- and cyclic imide groups. Further examples of protected amino groups are described by Greene and Wuts in Protective Groups in Organic Synthesis, 2^{nd} edition (John Wiley & Sons, New York, 1991).

The term “modified amino” as used herein includes the terms “protected amino,” “amine protecting group,” “alkylacylamino,” “acylamino” and “carboxamido”.

The term “modified hydroxyl” as used herein includes the terms “protected hydroxyl”, “hydroxyl protecting group”, “protected hydroxymethyl,” “alkoxy,” “aryloxy,” “acyl,” “carboxy esters,” and “acyloxy”

The term “protected hydroxyl”, “hydroxyl protecting group” and “protected hydroxymethyl” as used herein refers to known hydroxyl protecting groups used in the synthetic organic chemistry art and include but are not limited to methoxymethyl (MOM), benzyloxymethyl (BOM), benzyl (Bn), Allyl (All), p-methoxybenzyl (PMB), t-butyldimethylsilyl (TBDMS), ester groups, such as, acetate (Ac), and chloroacetate and benzoate (Bz). Further examples of protected hydroxyl groups are described by Greene and Wuts in Protective Groups in Organic Synthesis, 2^{nd} edition (John Wiley & Sons, New York, 1991).

Prodrugs

Prodrugs contain a chemical moiety, e.g., an amide or phosphorus group whose function is to endow enhanced solubility and/or stability to the attached drug so that it can be effectively preserved/delivered to a host. Once in the body, the prodrug is typically acted upon by an enzyme in vivo, e.g., an esterase, amidase, or phosphatase, to liberate/generate the more pharmacologically active drug. This activation may occur through the action of an endogenous host enzyme or a non-endogenous enzyme that is administered to the host preceding, following, or during administration of the prodrug. Prodrug use in general is further discussed, e.g., in U.S. Pat. No. 5,627,165, as well as in Pathak et al., Enzymic protecting group techniques in organic synthesis, Stereosel. Biocatal. 775-797 (2000), and is otherwise well-known in the art, although not to Applicants' knowledge using the specific compounds and compositions claimed herein.

Ideally, the prodrug should be converted to the original drug as soon as the goal is achieved, followed by the subsequent rapid elimination of the released derivatizing group. The term can also mean a nonspecific chemical approach to mask undesirable drug properties or improve drug delivery. For example, many therapeutic drugs have undesirable properties that may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, etc.). The prodrug approach, a chemical approach using reversible derivatives, can be useful in the optimization of the clinical application of a drug.

Pharmaceutical Compositions/Formulations, Dosaging, and Modes of Administration

Those of ordinary skill in the art are familiar with formulation and administration techniques, e.g., as discussed in Goodman and Gilman's The Pharmacological Basis of Therapeutics, current edition; Pergamon Press; and Remington's Pharmaceutical Sciences (current edition.) Mack Publishing Co., Easton, Pa. These techniques can be employed in appropriate aspects and embodiments of the invention.

The compounds utilized in the methods of the instant invention may be administered either alone or in combination with pharmaceutically acceptable carriers, excipients or diluents, in a pharmaceutical composition, according to standard pharmaceutical practice. The compounds can be administered orally or parenterally, including the intravenous, intramuscularly, intraperitoneal, subcutaneous, rectal and topical routes of administration.

For example, the therapeutic or pharmaceutical compositions of the invention can be administered locally to the area in need of treatment. This may be achieved by, for example, but not limited to, local infusion during surgery, topical application, e.g., cream, ointment, injection, catheter, or implant, said implant made, e.g, out of a porous, non-porous, or gelatinous material, including membranes, such as sialastic membranes, or fibers. The administration can also be by direct injection at the site (or former site) of a tumor or neoplastic or pre-neoplastic tissue.

Still further, the therapeutic or pharmaceutical composition can be delivered in a vesicle, e.g., a liposome (see, for example, Langer, 1990, Science, 249:1527-1533; Treat et al., 1989, Liposomes in the Therapy of Infectious Disease and Cancer, Lopez-Bernstein and Fidler (eds.), Liss, N.Y., pp. 353-365).

The pharmaceutical compositions used in the methods of the present invention can be delivered in a controlled release system. In one embodiment, a pump may be used (see, Sefton, 1987, CRC Crit. Ref. Biomed. Eng. 14:201; Buchwald et al., 1980, Surgery, 88:507; Saudek et al., 1989, N. Engl. J. Med., 321:574). Additionally, a controlled release system can be placed in proximity of the therapeutic target. (see, Goodson, 1984, Medical Applications of Controlled Release, Vol. 2, pp. 115-138).

The pharmaceutical compositions used in the methods of the instant invention can contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinylpyrrolidone or acacia, and lubricating agents, for example, magnesium stearate, stearic acid or talc. The tablets may be uncoated or they may be coated by known techniques to mask the taste of the drug or delay disintegration and absorption in the gastrointestinal tract and thereby provide a sustained action over a longer period. For example, a water-soluble taste masking material such as hydroxypropylmethyl-cellulose or hydroxypropylcellulose, or a time delay material such as ethyl cellulose, or cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions can contain the active material in admixture with excipients suitable for the manufacture of aqueous suspensions. Such excipients can act as suspending agents and include, e.g., sodium carboxymethylcellulose, methylcellulose, hydroxypropylmethyl-cellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant, e.g., butylated hydroxyanisol, alpha-tocopherol, or ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of anti-oxidant(s).

The pharmaceutical compositions used in the methods of the instant invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of composition suitable for use as an inhalant

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents, which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The aminoglycosides used in the methods of the present invention may also be administered in the form of suppositories for rectal administration of the drug. These compositions can be prepared by mixing the inhibitors with a suitable non-irritating excipient, which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing an aminoglycoside can be used. As used herein, topical application can include mouth washes and gargles.

The compounds used in the methods of the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The methods and compounds of the instant invention may also be used in conjunction with other well known therapeutic agents that are selected for their particular usefulness against the condition that is being treated. For example, the instant compounds may be useful in combination with known anti-cancer and cytotoxic agents.

Preferably the pharmaceutical preparation is in unit dosage form. In such form, the preparation is subdivided into unit doses containing appropriate quantities of the active component, e.g., an amount that is effective to achieve the desired purpose.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the compounds used in the methods of the present invention and, if applicable, other chemotherapeutic agents will be regulated according to the judgment of the attending clinician (physician) considering such factors as age, condition and size of the patient as well as severity of the disease being treated.

In general, compounds of the invention and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician. The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the proliferative disease, the condition of the patient, and the actual choice of compounds used.

The determination of the order of administration, and the number of repetitions of administration of each therapeutic agent during a treatment protocol, is well within the knowledge of the skilled physician after evaluation of the disease being treated and the condition of the patient.

This invention also relates to aminoglycoside compounds that are useful as prophylaxis or antibacterial agents in mammals avian, fish and reptile species as well as in cell culture. Furthermore, the compounds of the present invention, based on their ability to interact with RNA are useful for treating viral infection, genetic disorders like muscular dystrophy, cystic fibrosis and cancer (Sucheck, S. J. and Wong, C.-H. Curr. Opin. Chem. Biol. 2000, 4, 678). The compounds can be administered by i.v. infusion for treatment of systemic infections, aerosolization for treatment of respiratory infection, and used in creams or drops for the topical treatment of skin, ear and eye infections.

Aminoglycosides

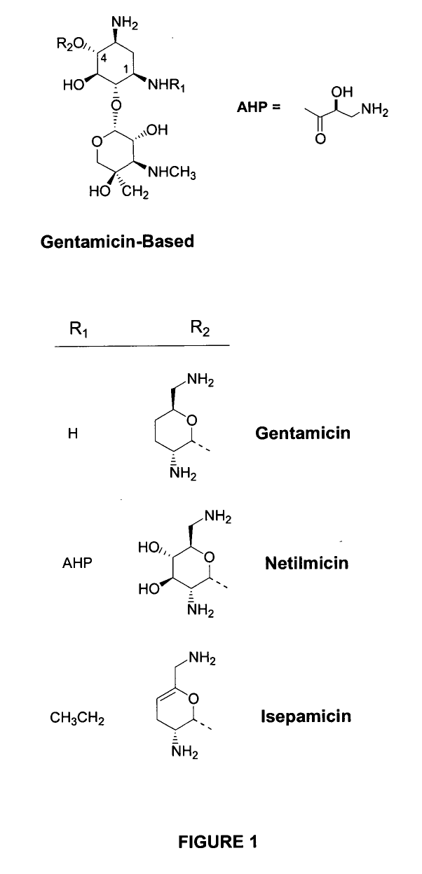

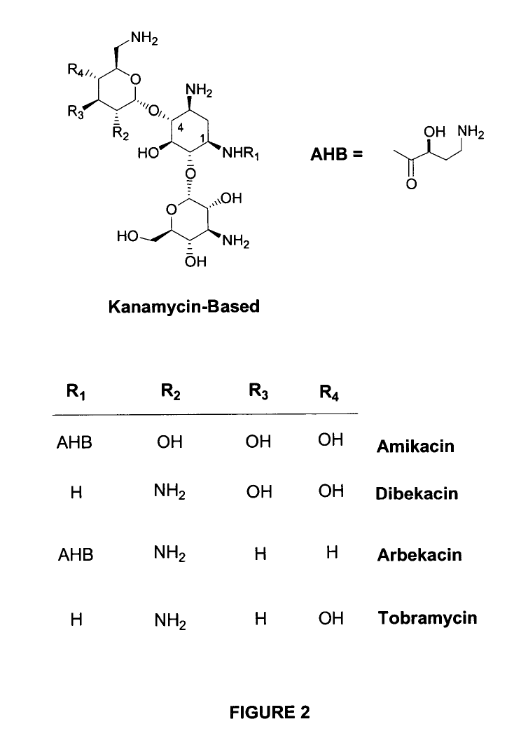

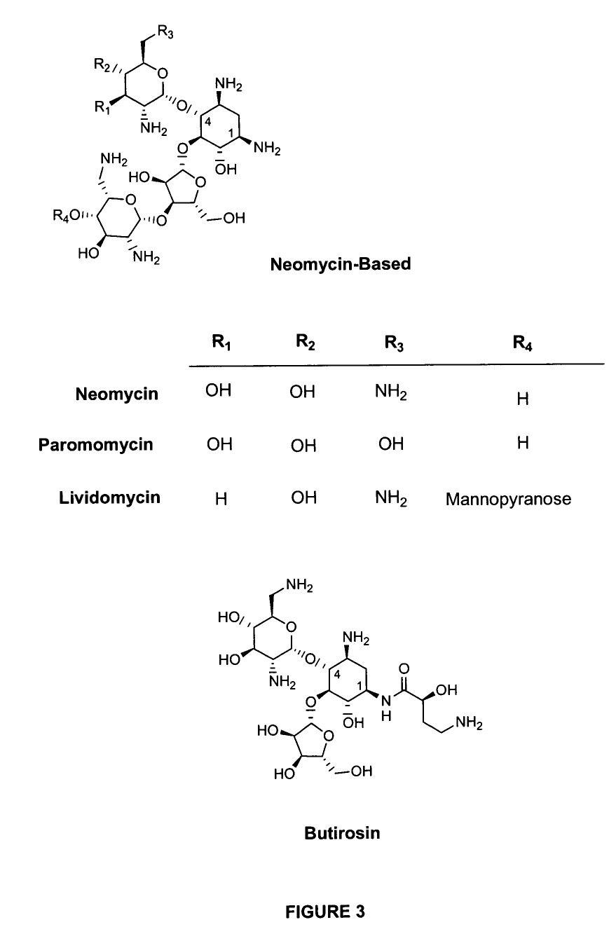

As shown in FIGS. 1, 2 and 3 the 2-deoxystreptamine (2-DOS) ring system is a common motif found in AMGs, such as, tobramycin, bekanamycin, gentamicins, arbekacin, netilmicin and dibekacin. In one embodiment of the present invention, “ring I” (the gycosyl group at the 4-position of the 2-DOS ring) is replaced with a novel glycosyl or disaccharide moiety that improves binding to its target RNA. Furthermore, the introduction of these novel carbohydrates makes the compounds of the present invention resistant to some aminoglycoside modifying enzymes.

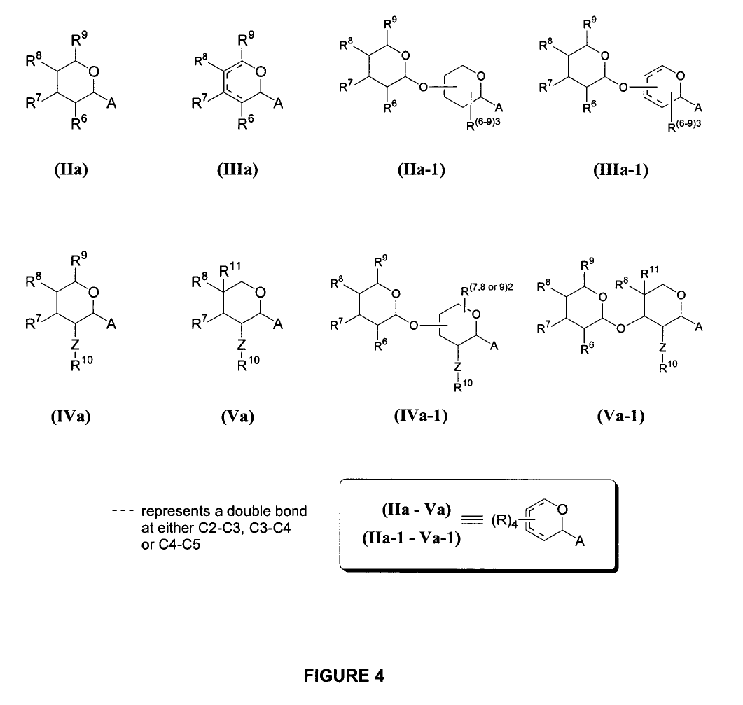

The structures (II), (II-1), (III), (III-1), (IV), (IV-1), (V) and (V-1) can be represented as a single structure as is illustrated in FIG. 4.

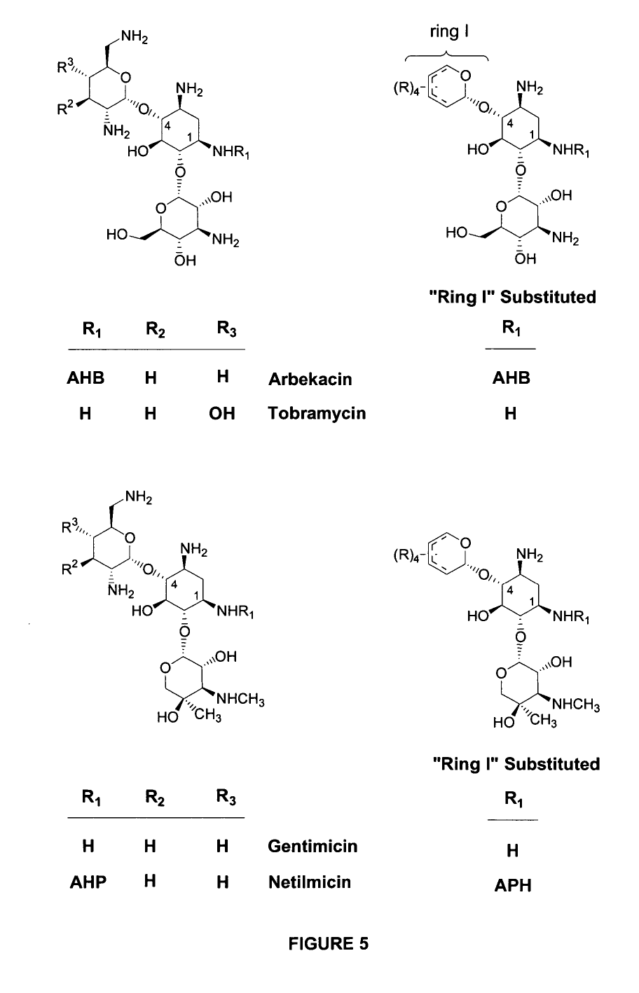

Tobramycin and gentamicin are aminoglycoside antibiotics that have a 4,6-substituted ring system as shown in FIG. 5. In one embodiment of the present invention, Ring I (the gycosyl ring at the 4-position of the 2-DOS ring) of tobramycin is replaced with a novel glycosyl or disaccharide moiety.

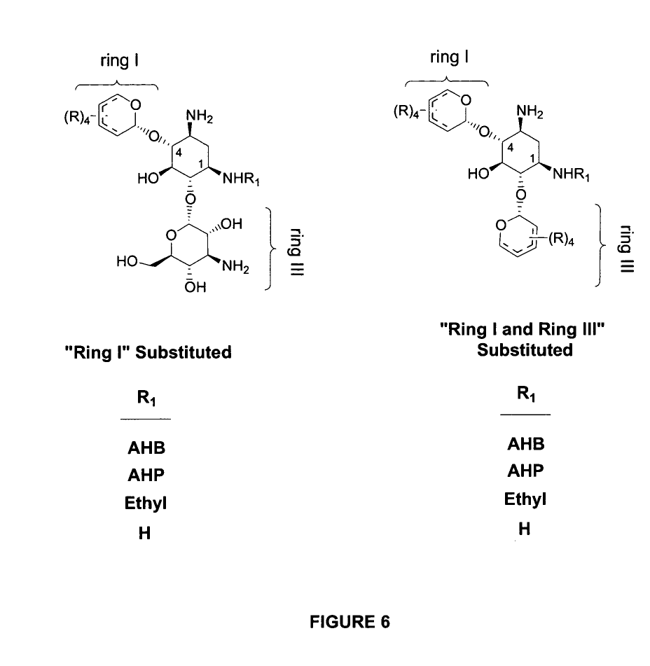

In another embodiment of the present invention, Ring I (the gycosyl ring at the 4-position of the 2-DOS ring) and Ring III (the gycosyl ring at the 6-position of the 2-DOS ring) of tobramycin or a related 4,6-substituted aminoglycoside are both replaced with a novel glycosyl or disaccharide moieties as shown in FIG. 6.

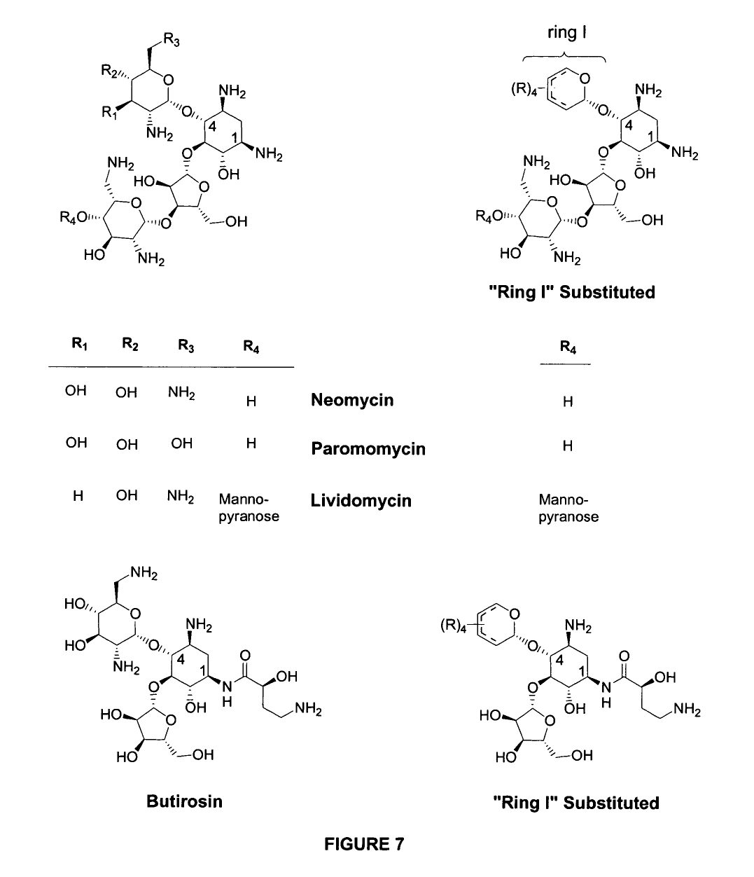

Neomycin and butirosin are aminoglycoside antibiotics that have a 4,5-substituted ring system as shown in FIG. 7. In one embodiment of the present invention, Ring I (the glycosyl ring at the 4-position of the 2-DOS ring) of neomycin is replaced with a novel glycosyl or disaccharide moiety.

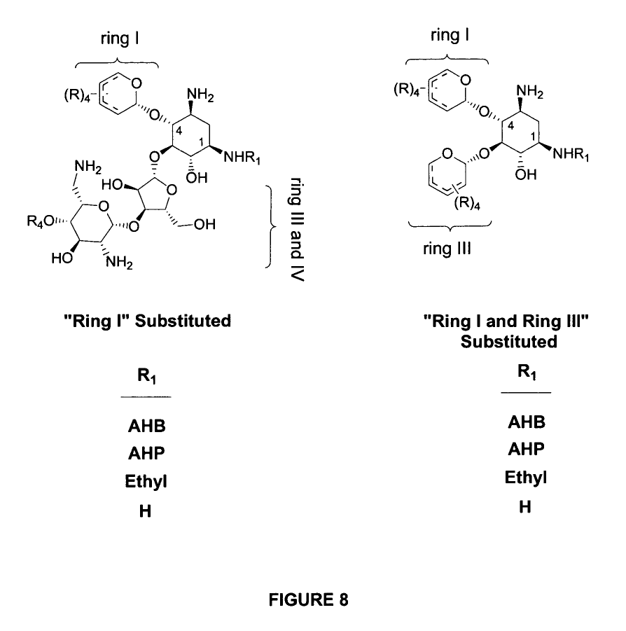

In another embodiment of the present invention, Ring I (the gycosyl ring at the 4-position of the 2-DOS ring) and Ring III or (Ring III and IV) (the gycosyl rings at the 5-position of the 2-DOS ring) of neomycin or a related 4,5-substituted aminoglycoside are both replaced with a novel glycosyl or disaccharide moieties as shown in FIG. 8.

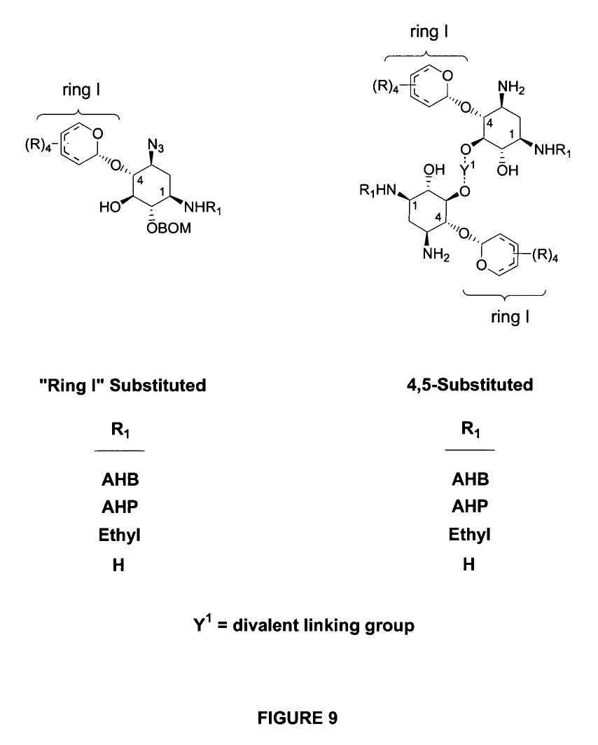

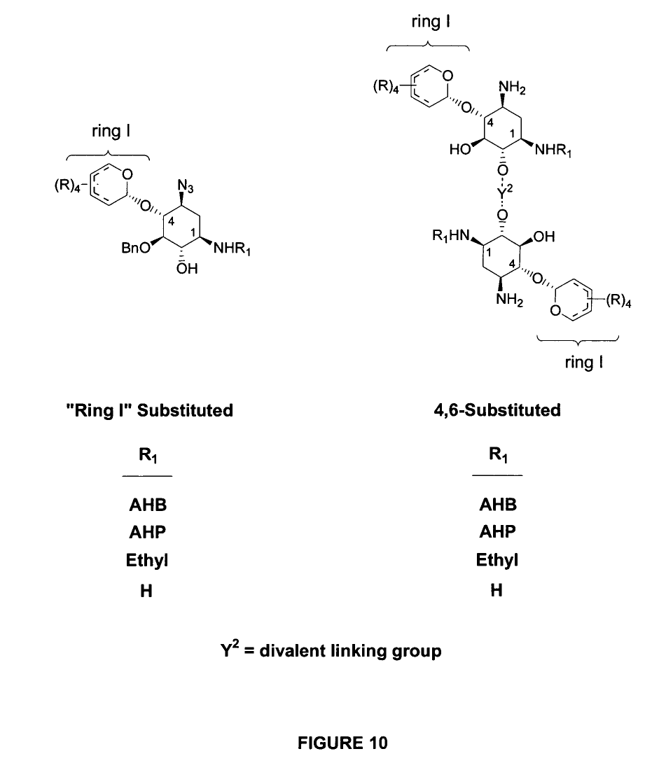

In another embodiment of the invention AMGs of the present invention have the naturally occurring glycosyl groups attached to the 2-DOS ring at the 4-position held constant or replaced by a novel glycosyl or disaccharide group; while, the gycosyl group at the 5- or 6-position is replaced by a linking group connected to a glycosyl group or a 2-DOS glycosylated at the 4-position as shown in FIG. 9 and FIG. 10, respectively.

In another aspect, aminoglycoside compounds of the invention have the general formula (I):

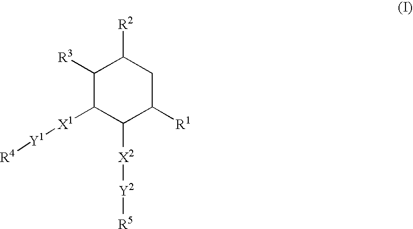

wherein, X, Y, Z and R¹ through R⁵ are as previously defined.

X¹ and X² are independently O, S or NH. In one embodiment X¹ and X² are O.

Y¹ and Y² are independently a bond or a divalent linking group. In one embodiment one of Y¹ or Y² is a divalent linking group and the other is a bond.

In one embodiment, one of Y¹ or Y² is a bond R⁴ and R⁵ is hydrogen, hydroxyl protecting or modified hydroxyl group and the other y1 or y2_is selected from, but not limited to:

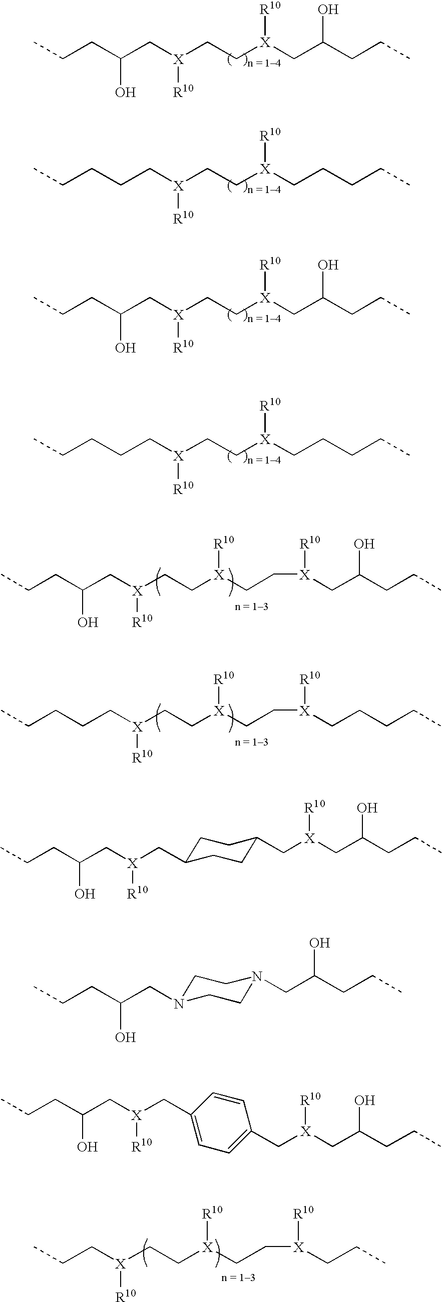

X can be independently O, S or N.

When X=N then R¹⁰ can be hydrogen, an alkyl group, an amine protecting group.

When X is not ═N then R¹⁰ can be a lone pair of electrons.

In another embodiment,

X¹ and X² are independently O, S or NH. In one embodiment X¹ and X² are O.

Y¹ and Y² are independently a bond or a divalent linking group. In one embodiment Y¹ and Y² are bonds.

In one embodiment, one of R⁴ and R⁵ is hydrogen, hydroxyl protecting or modified hydroxyl group when one of Y¹ or Y² is a bond and the other is selected from a group consisting of formula (II), (II-1), (III), (III-1), (IV), (IV-1), (V) or (V-1).

R¹ and R² are independently amino or a protected amino or modified amino. Protected amino groups are known and used in synthetic organic chemistry and include but are not limited to formyl, acetyl, trichloroacetyl, trifluoroacetyl, benzoyl (Bz), benzyloxycarbonyl (CBz), fluorenylmethyloxycarbonyl (Fmoc), t-butylcarbonyl (t-Boc) and trichloroethanecarboxyl (Troc). One protected amino group is an azide (N₃) which can be converted back to an amine using catalytic hydrogenolysis or trimethylphosphine and water (Wong, C.-H.; Sucheck, S. PCT Int. Appl. 2001, 47 pp). In another embodiment R¹ and R² are both N₃. In another embodiment both R¹ and R² are NH₂. In another embodiment R² is NH₂ and R¹ is an —NHCH₂CH₃ or an amide of a α-hydroxy-ω-aminoalkanoyl group of the formula:

—NHCO—CH(OH)—(CH₂)ₙ—NH₂ (n=2-6)

or an amide resulting from amidation of an amine with a d- or/-amino acid, or a peptide;

or a cyclic or open amidine or guanidine group.

R³ is saccharide or disaccharide selected from the group consisting of the formula (II) or (III) as previously defined. Substituent groups for the above saccharide R⁶, R⁷, R⁸ and R⁹ include but are not limited to halogen (fluoro, chloro, bromo, iodo), hydroxyl (OH), hydroxymethyl (CH₂OH), aminomethyl (CH₂NH₂), thiol (SH), hydrogen (H), alkyl, carboxamide (CONHR were R is hydrogen, alkyl, carbocyclic, aryl or a heteroaryl group), aldehyde (CHO), keto (C═O), nitrile (CN), amidino (C(NR)NRR where R can be independently hydrogen, aliphatic, carbocyclic, aryl or a heteroaryl goup), guanidino (NR—C(NR)NRR where R can be independently hydrogen, aliphatic, carbocyclic, aryl or a heteroaryl group), trifluoromethyl (CF₃) trifluomethoxy (OCF₃), alkoxy, aryloxy, S-alkyl thioether, S-aryl, or amino (NH₂), including NH-alkyl, N-(alkyl)₂, NH-aryl and nitro (NO₂), aryl and heteroaryl or R⁶, R⁷, R⁸ and R⁹ can be independently another mono- or disaccharide (II), including disaccharides (II-1) and (III-1).

In one embodiment, the saccharides (I) or (II) are substituted with one or more of the groups selected from the group consisting of F, Cl, Br, 1, OH, SH, H, CN, NO₂, CHO, OCF₃, CF₃, NH₂, alkyl, NH-aryl and aryl, heteroaryl, aryloxy, keto, amidino, guanidine, OCONHR¹², COR², OR¹², S—R², S-aryl, NHR¹² N—(R¹²)₂; wherein, R¹² is hydrogen, C₁₋₈ alkyl (wherein alkyl is straight, branched carbocyclic (saturated or unsaturated), aryl or a heteroaryl group). In another embodiment (II) and (III) are substituted with F, OH, H, NH₂, NHR¹², N—(R¹²)₂; keto, amidine and guanidine. In another embodiment R⁶, R⁷, R⁸ and R⁹ can be independently another saccharide (II) to form, but not limited to disaccharides (II-1) and (III-1).

In another embodiment R³— is selected from:

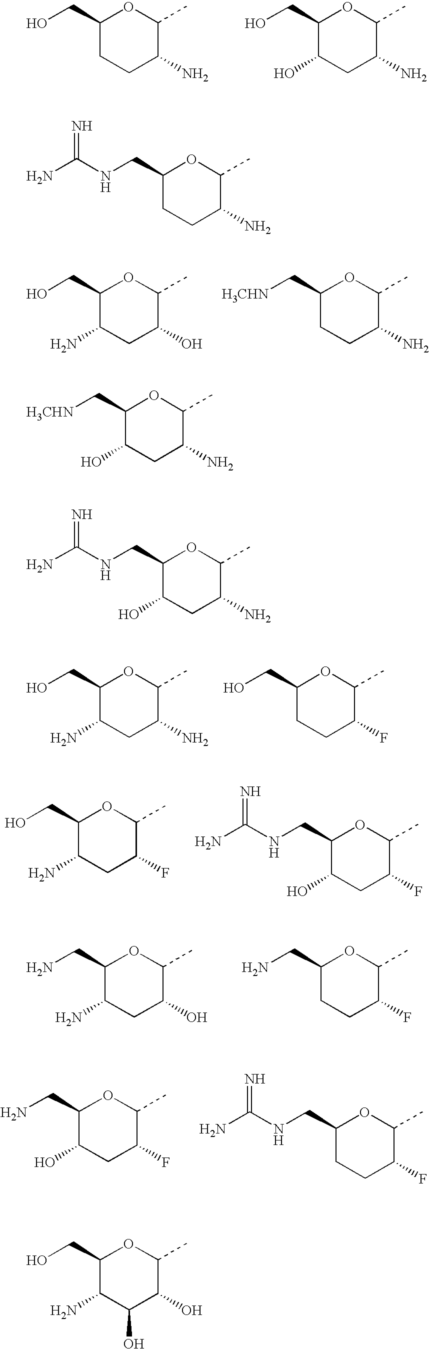

In another embodiment R³— is selected from:

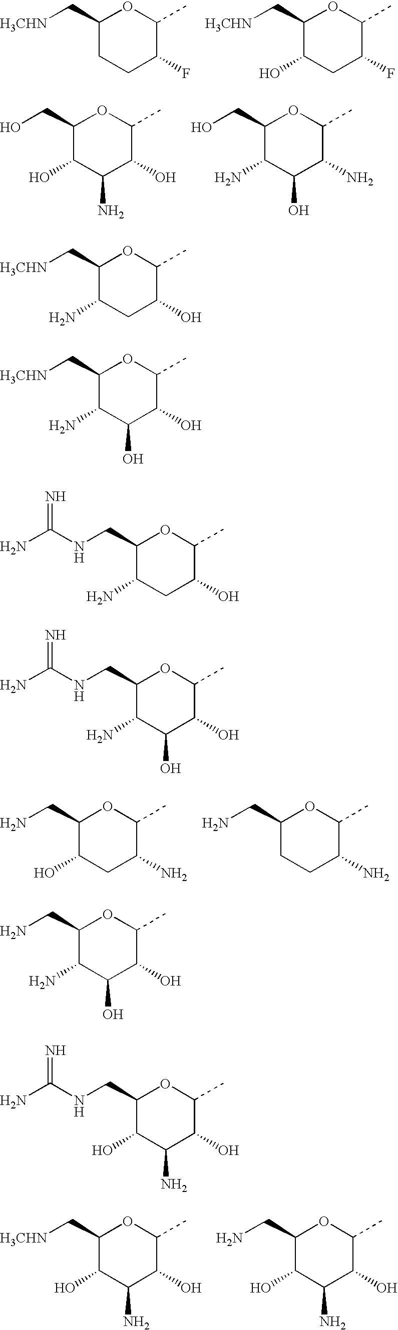

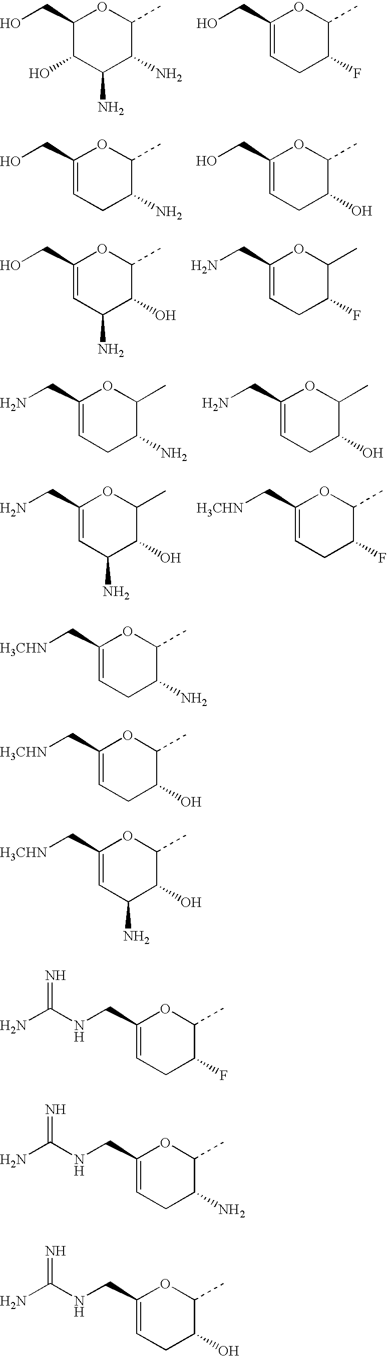

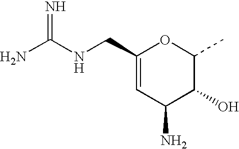

In another embodiment R³— is selected from:

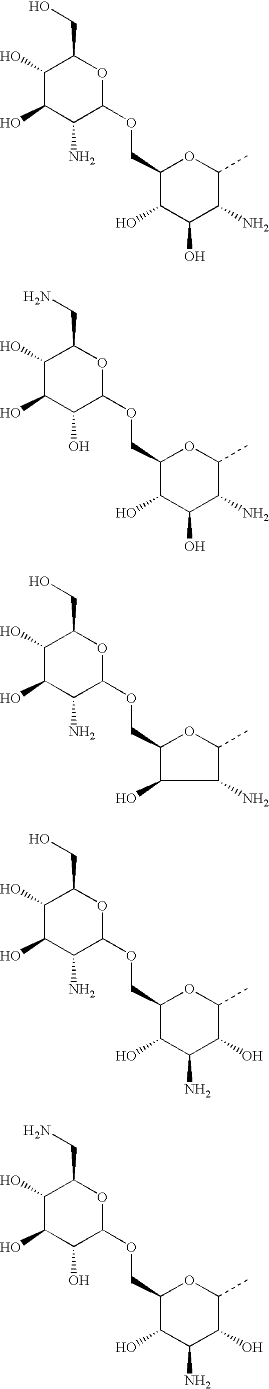

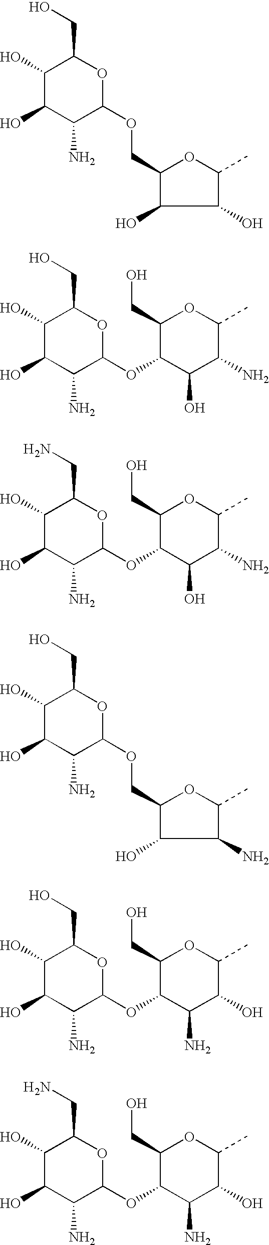

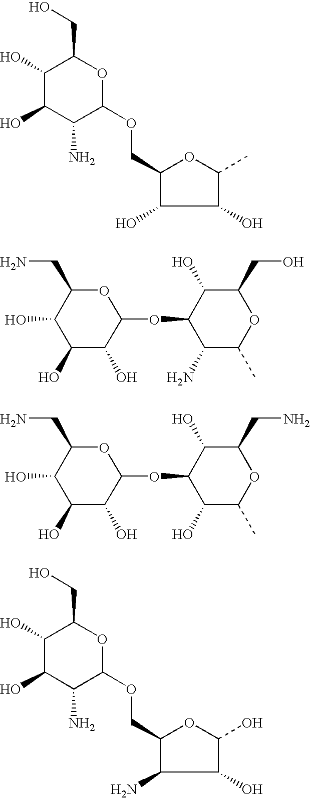

In another embodiment R³— is selected from:

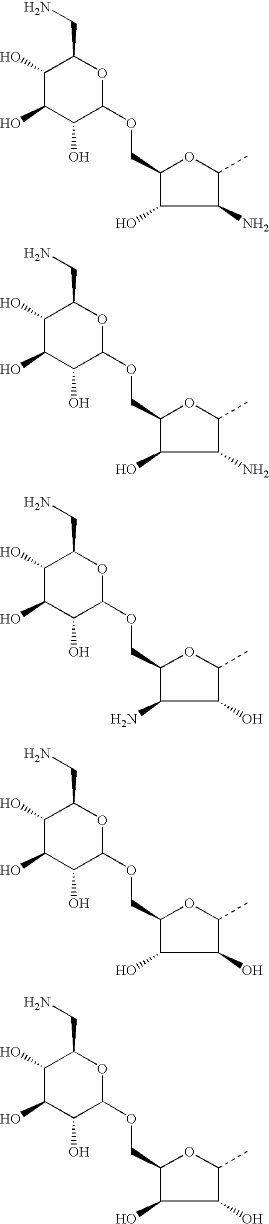

One of R⁴ and R⁵ is hydrogen, hydroxyl protecting or modified hydroxyl group. Any hydroxyl protecting or modified hydroxyl group known in the art of synthetic organic chemistry can be used. In one embodiment one hydroxyl group is benzyl or benzyloxymethoxy protected. The other of R⁴ and R⁵ is selected from a group consisting of formula (II), (II-1), (III), (III-1) (IV), (IV-1), (V), (V-1), (VI) or (VII):

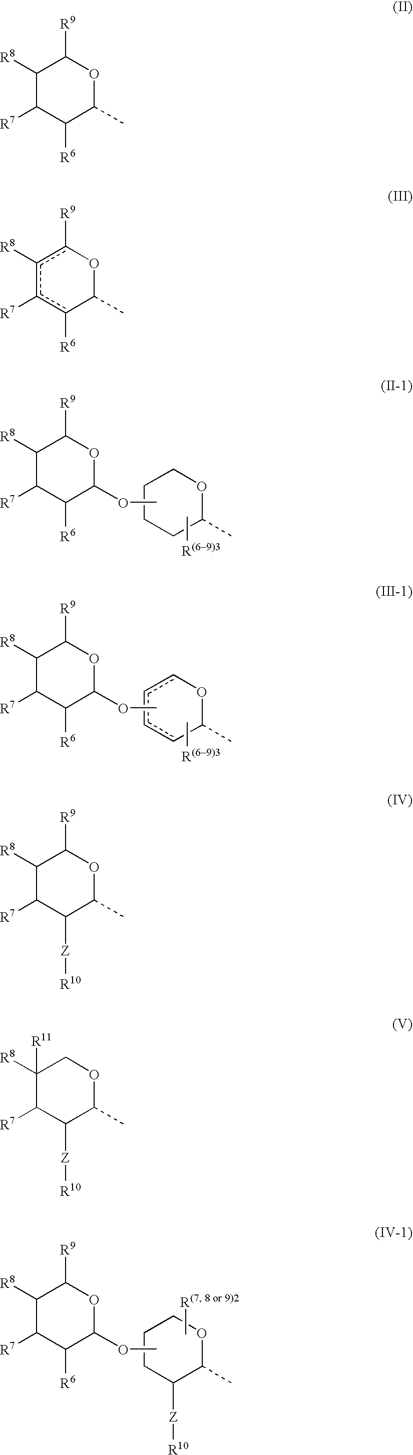

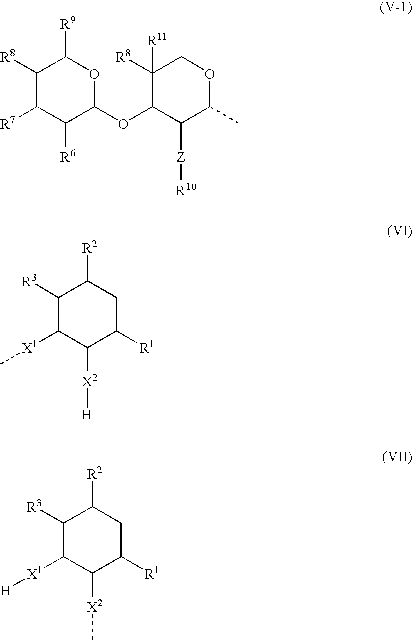

wherein,

substituents R¹, R², R³, R⁶, R⁷, R⁸ and R⁹ for the above structure (II), (II-1), (III), (III-1) (IV), (IV-1), (V), (V-1), (VI) and (VII) are as previously described.

X¹ and X² are independently O, S or NH.

Z can be O, S or NH.

R¹⁰ can be a hydrogen, alkyl group, amine protecting group, modified amino, oxygen, hydroxyl protecting or modified hydroxyl group, and

R¹¹ can be a hydrogen, halogen or alkyl group.

In another embodiment one of R⁴ and R⁵ is the group of formula (IV). In this instance R⁷ and R⁸ are hydrogen, a halogen, hydroxyl; protected-hydroxyl, amino, protected amino, modified amino, guanidine or amidine.

R⁹ is hydrogen, hydroxyl, protected hydroxyl, amino, protected amino, modified amino, hydroxymethyl, protected hydroxymethyl, aminomethyl, protected aminomethyl, keto, halogen, NHR¹², N—(R¹²)₂, carbamate (OCONHR¹²), guanidine, or amidine.

Z is NH.

In another embodiment R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen. R⁷, R⁸ and R⁹ can be independently another saccharide (II) to form disaccharides (IV-1).

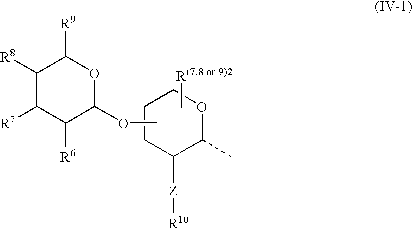

R¹⁰ can be a hydrogen, alkyl group, amino, amine protecting group or modified amino. In another embodiment R¹⁰ can be a hydrogen, alkyl group or Z-R¹⁰ together can form an amidine, guanidine or modified amino. In another embodiment the group of the formula (IV) has the stereochemistry as defined by the formula (IVb).

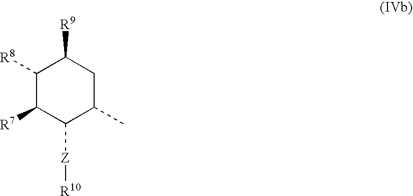

In another embodiment, one of R⁴ and R⁵ is the group or the formula (V). In this instance R⁷ and R⁸ are hydrogen, a halogen, hydroxyl; protected hydroxyl, modified hydroxyl, amino, protected amino, modified amino, guanidine or amidine. In another embodiment R⁷ and R⁸ can be independently another saccharide (II) to form disaccharides (V-1).

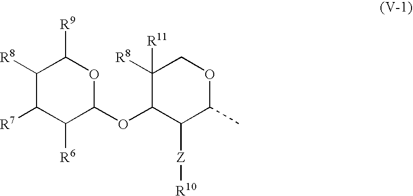

Z is NH.

R¹⁰ can be a hydrogen, alkyl group, amine protecting group or Z-R¹⁰ together can form an amidine, guanidine or modified amino, and

R¹¹ can be a hydrogen, halogen or alkyl group.

In another embodiment the group of the formula (V) has the stereochemistry as defined by the formula (Vb).

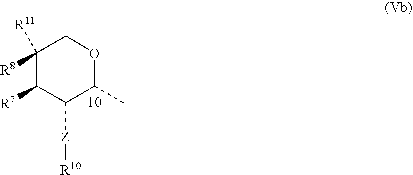

In another embodiment, one of R⁴ and R⁵ is the group or the formula (VI).

In this instance R¹, R² and R³ are as previously described.

X¹ and X² are independently O, S or NH. In one embodiment X¹ and X² are O.

Y¹ and Y² are independently a bond or a divalent linking group. In one embodiment Y¹ or Y² is a divalent chain. In another embodiment Y¹ or Y² is as previously described.

In another embodiment the group of the formula (VI) and (VII) has the stereochemistry as defined by the formula (VIa) and (Vila), respectively.

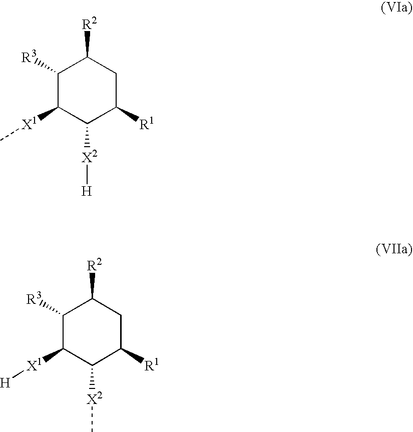

Compounds of the invention may be prepared according to established synthetic organic chemistry techniques. In another method, selectively protected 2-DOS is prepared chemo-enzymaticly starting from neomycin B (Alper, P. B.; Hendrix, M.; Sears, P.; Wong, C.-H. J. Am. Chem. Soc. 1998, 120, 1965). Selectively protected 2-DOS is glycosylated at the 4-position with a natural or unnatural glycosyl donor defined by R³. The glycosylation can be accomplished by one of three general methods: 1) Glycosylation with a fully protected saccharide, 2) glycosylation with a fully protected disaccharide or, 3) glycosylation with a mixture of fully protected saccharide and a saccharide with one free hydroxyl. The third method can also by described as glycosylation by programmable one-pot synthesis. The general method of programmable one-pot synthesis has been described (Baasov, T. et al., WO0009527, 2000, 109 pp; Koeller, K. M. and Wong, C.-H. Chem. Rev. 2000, 100, 4465-4493). The glycosylated 2-DOS intermediate is subsequently glycosylated at the 5- or 6-position with various glycosyl donors that are either natural or unnatural. The glycosyl donors can include disaccharides and the disaccharide can be formed in situ using the programmable one-pot synthesis method. In some cases the 5- or 6-position is derivatized with divalent group, generally a diamine, defined as Y¹ or Y². X—R⁴ or X—R⁵ are a 2-DOS that has been glycosylated at the 4-position and is then linked to the divalent group Y¹ or Y² through the 5- or 6-position. The general method has been described (Wong, C.-H. and Sucheck, S. WO0180863, 2001, 47 pp; Sucheck, S. J.; Wong, A. L.; Koeller, K. M.; Boehr, D. D.; Draker, K.; Sears, P.; Wright, G. D.; Wong, C.-H. J. Am. Chem. Soc. 2000, 122, 5230).

Preparation of 5,6-O-acetyl-1,3-diazido-2-deoxystreptamine (compound 5, Scheme 3) was carried out according the procedure of Alper et al. (Alper, P. B.; Hendrix, M.; Sears, P.; Wong, C.-H. J. Am. Chem. Soc. 1998, 120, 1965). The remaining free hydroxy at the 4-position is used as a glycosyl acceptor for appending various glycosyl donors to the 2-DOS ring.

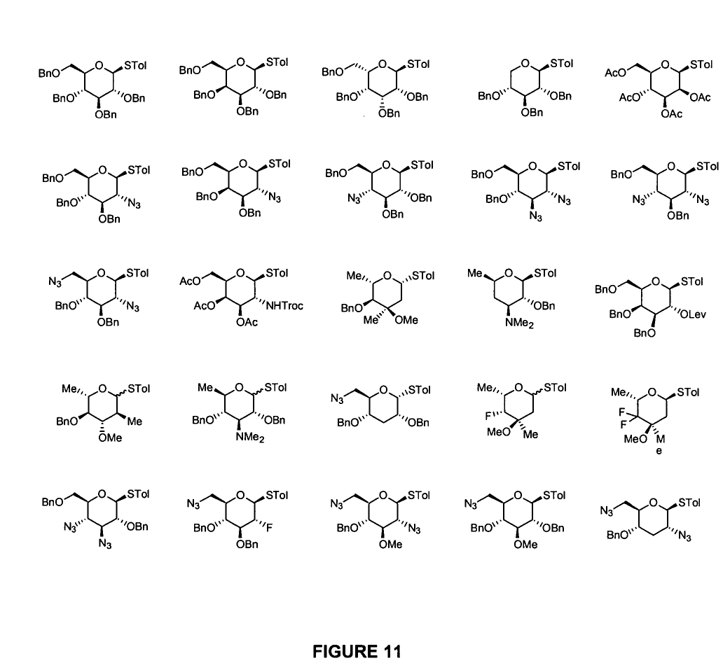

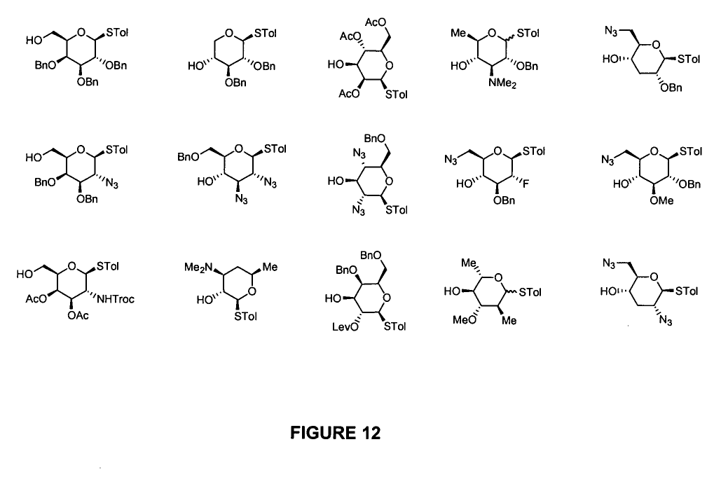

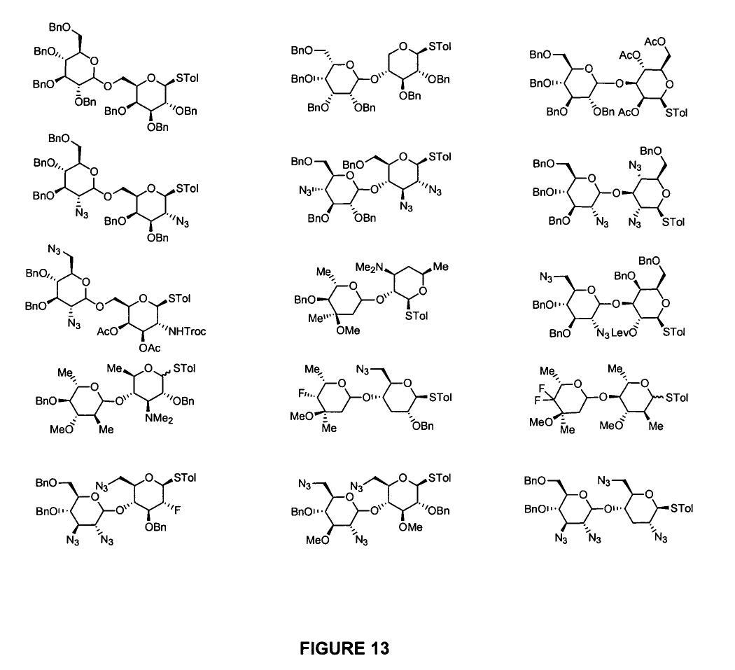

The glycosyl donor is generally a thioglycoside activated by N-iodosuccinimide (NIS)/trifluormehtane sulfonic acid (TfOH) or dimethyl(methylthio)sulfonium triflate (DMTST). Representative glycosyl donors are shown in FIG. 11 and FIG. 13; glycosyl donors can be selected from FIG. 11 and FIG. 12 to accomplish a programmable one-pot synthesis of a disaccharide; however, glycosyl donors are not limited to these examples. The resulting product is purified and the c/p anomers are separated by silica gel column chromatography.

Once the 2-DOS ring is derivatized at the 4-position, the hydroxyl protecting groups at the 5- and 6-position may be removed leaving free hydroxyl groups for further derivatization or another glycosylation. For example, the acetates can be removed by basic hydrolysis.

The deproteded gycosylated 2-DOS ring may be glycosylated with a glycosyl donor (single disaccharide, disaccharide or by programmable one-pot synthesis) selected from glycosyl donors including but not limited to:

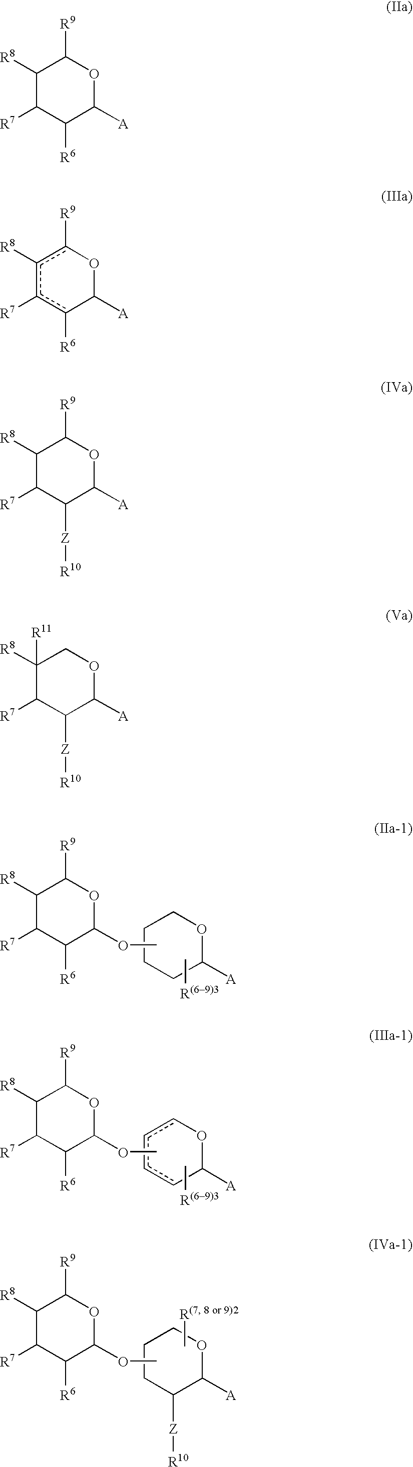

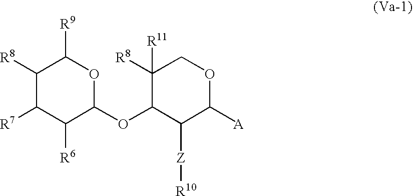

Wherein, A is a carbohydrate-activating group. In one embodiment a leaving group may be a thio-leaving group such as SR wherein R is alkyl, aryl or substituted aryl. The glycosyl donor is generally activated by N-iodosuccinimide (NIS)/trifluormehtane sulfonic acid (TfOH) or dimethyl(methylthio)sulfonium triflate (DMTST). The general method for the gycosylation of 2-DOS is well established (Swayze, E.; Griffey, R.; Ding, Y.; Mohan, V. PCT Int. Appl. 2001, 57 pp; Alper, P. B.; Hendrix, M.; Sears, P.; Wong, C.-H. J. Am. Chem. Soc. 1998, 120, 1965).

Gycosyl donors according to the invention include pyranose or furanose sugars wherein the hydroxyl groups may be replaced with hydrogen, amino, protected amino or modified amino groups. The donors of the present invention are prepared starting from naturally occurring carbohydrates, e.g., glucose. Prior to their use, they may require proper functional group protection. The relative reactivity of the various hydroxyl and amino groups of carbohydrates has been established. Such differences in reactivity permit the preparation of the mentioned saccharides used for the preparation of the compounds of the present invention.

The protecting groups of the intermediates are removed using standard conditions. In many cases protecting groups can be removed orthogonally or all at once. This allows for further derivatization of amines and hydroxyl functional groups. For example, amines are reacted with activated esters, isonitriles and metylthiopseudoureas to form amides, ureas, guanidines and amidines. Alcohols can be reacted with activated esters or isonitriles to form esters and carbamates. Alcohols can be reacted with tosylchloride to form tosylates, which can be displaced with azides or alkylamines to form azido and alkylamines, respectively. The resulting azides can be deprotected to provide the corresponding amines.

In the method of Scheme 1, commercially available 4,6-substituted AMGs, such as, tobramycin, bekanamycin, gentamicins, arbekacin, netilmicin and dibekacin are chemically derivatized to provide the disaccharide cores 1 as is described in Example 1. The method involves reactions of present AMGs with triflic azide in the presence of copper (II) or zinc (II) catalyst followed by benzylation with benzyl bromide. The benzylated intermediate is chemically derivatized by acidic methanolysis or hydrolysis to provide the protected pseudodisaccharide cores 1 of the present invention. The ease of this process to synthesize advanced aminoglycoside intermediates with a free hydroxyl at the 6-position is noteworthy. The overall yield over three steps can be as high as 80%.

In the method of Scheme 2, as is described in Example 2a-c, glycosylation of compound 1 at the 6-position with gycosyl donors (IIa) (IIIa) (IVa) or (Va) provides a pseudodissacharide. The glycosyl donor is generally a thioglycoside activated by N-iodosuccinimide (NIS)/trifluormehtane sulfonic acid (TfOH) or dimethyl(methylthio)sulfonium triflate (DMTST). The resulting product is purified and the α/β anomers are separated by silica gel column chromatography. In the most common case the hydroxyl and amine protecting groups were azido and benzyl, respectively. The protecting groups were generally deprotected by hydrogenolysis using 1 atm. H₂ over 20% Pd(OH)₂/C to afford compounds 3. The deprotection conditions are not limited to these conditions. The primary amine of Ring I in structure 3 could be modified with amino acids, guanidinylating or amidinylating agents and isonitriles etc. to provide 4, as shown in Example 3. The primary amine or hydroxyl of Ring III could be similarly modified.

Similarly, in the method of Scheme 3, glycosylation at the 6-position of compound 1 with gycosyl donors (IIa-1) (IIIa-1) (IVa-1) or (Va-1) provides a pseudotrissacharide 3 as well. Likewise 4,5-linked analogues of 2-DOS (FIG. 8) can be prepared from neomycin B, paromomycin, lividomycin and butirosin by transforming into the perazido/perbenzyl intermediate by hydrolysis as shown in Example 4 and Example 5. The methods for the preparation of 4,5-linked analogues of 2-DOS have been previously described (Wong, C.-H.; Sucheck, S. PCT Int. Appl. 2001, 47 pp; Sucheck, S. J.; Wong, A. L.; Koeller, K. M.; Boehr, D. D.; Draker, K.; Sears, P.; Wright, G. D.; Wong, C.-H. J. Am. Chem. Soc. 2000, 122, 5230).

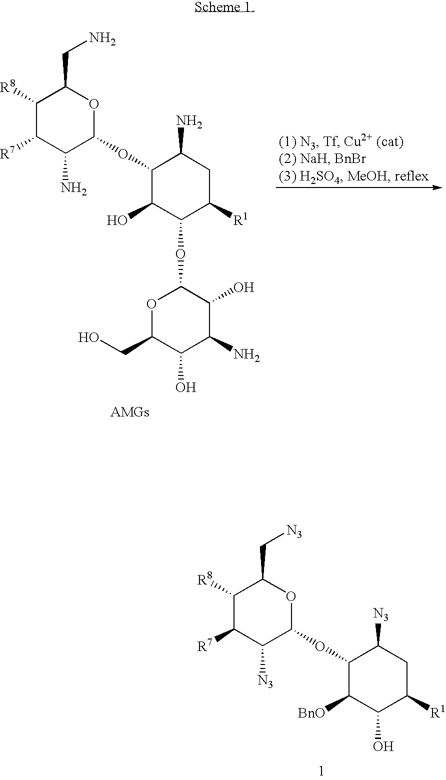

wherein: in the AMGs, R¹=—NH₂, —NHCH₂CH₃, —NHCO—CH(OH)—(CH₂)ₙ—NH₂ (n=2-12); R⁷═H or OH, R═H or OH

wherein: in structure 1, R¹=—N₃, —NBnCH₂CH₃, —NBnCO—CH(OBn)-(CH₂)ₙ—N₃ (n=2-12), R⁷=H or OBn, R═H or OBn

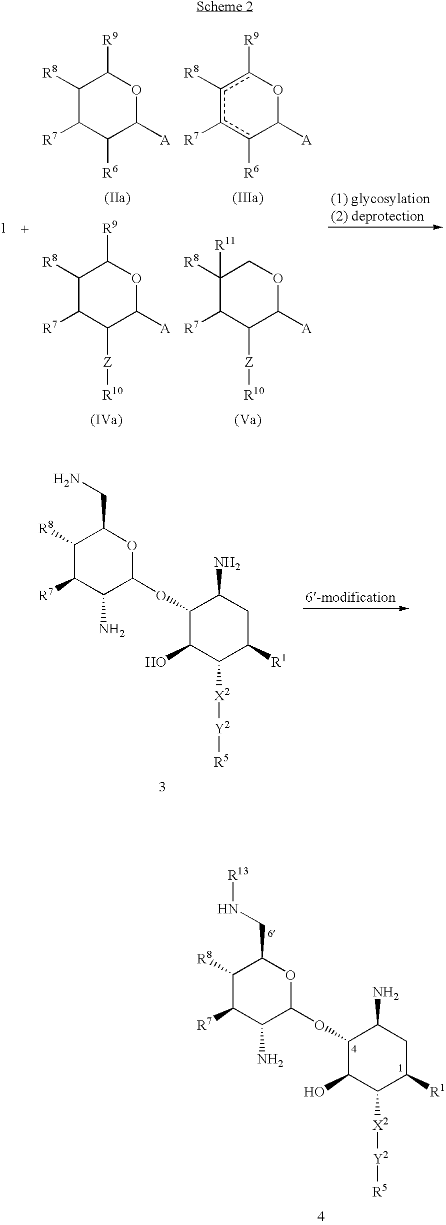

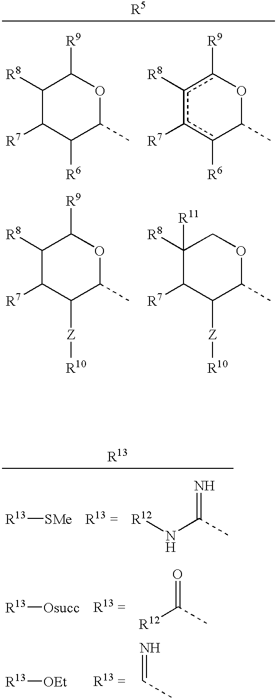

wherein: in structure 3 and 4, R¹=—NH₂, —NHCH₂CH₃, —NHCO—CH(OH)—(CH₂)ₙ—NH₂ (n=2-12); R⁶, R⁷, R⁸, R⁹ are as described in the text.

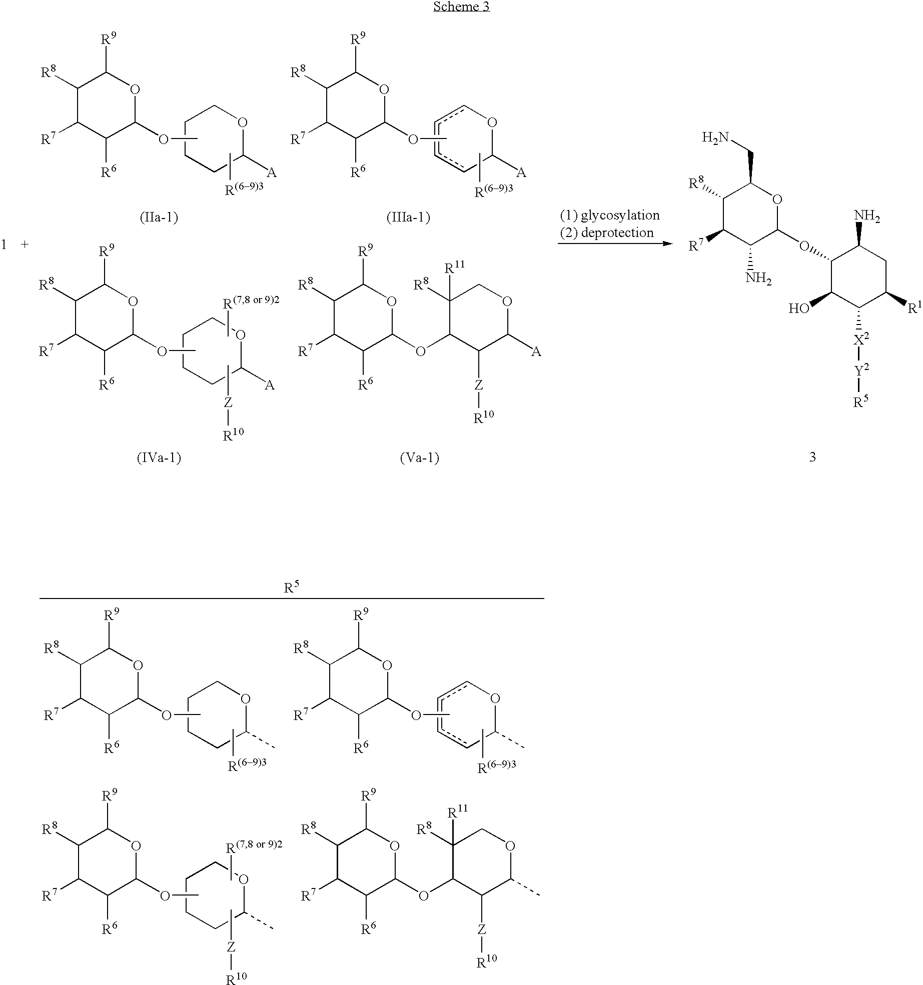

wherein: in structure 3, R¹=—NH₂, —NHCH₂CH₃, —NHCO—CH(OH)—(CH₂)ₙ—NH₂ (n=2-12); R⁶, R⁷, R⁸, R⁹ are as described in the text.

In the method of Scheme 4, glycosylation at the 6-position of pseudodisaccharide 1 using the method of programmable one-pot synthesis provides a pseudotrissacharide 3 as shown in Example 6 and Example 7. Two gycosyl donors are selected from (IIa) (IIIa) (IVa) or (Va). One glycosyl must have no reactive hydroxyls and must be easily activated. The second glycosyl donor must have one free hydroxyl and be approximately one tenth as reactive as the gylcosyl donor with no free hydroxyls (Baasov, T. et al., WO00/09527, 2000, 109 pp; Koeller, K. M. and Wong, C.-H. Chem. Rev. 2000, 100, 4465-4493). Similarly, as in the method of Scheme 3, glycosylation at the 6-position of compound 1 with gycosyl donors (IIa-1) (IIa-1) (IVa-1) or (Va-1) provides a pseudotrissacharide 3 as shown in Example 6b. Likewise 4,5-linked analogues of 2-DOS (FIG. 8) can be prepared from pseudodisaccharide derived from neomycin B, paromomycin, lividomycin and butirosin by transforming into the perazido/perbenzyl intermediate by hydrolysis as shown in Example 4 and Example 5.

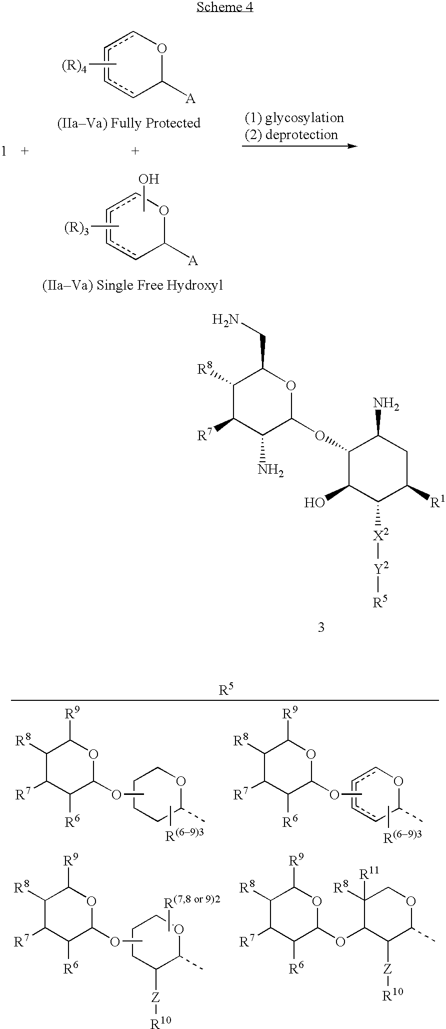

wherein: in structure 3, R¹=—NH₂, —NHCH₂CH₃, —NHCO—CH(OH)—(CH₂)ₙ—NH₂ (n=2-12); R⁶, R⁷, R⁸, R⁹ are as described in the text.

In the method of Scheme 5, a pseudodisaccharide core 6 of the present invention can be prepared from glycosylation of a suitably protected 2-DOS 5 with suitably protected glycosyl donors (IIa) (IIa) (IVa) and (Va) or disaccharide donors (IIa-1) (IIa-1) (Iva-1) and (Va-1). Representative glycosyl donors are shown in FIGS. 11 and 13, respectively; however, glycosyl donors are not limited to these. The glycosyl donor is generally a thioglycoside activated by N-iodosuccinimide (NIS)/trifluormehtane sulfonic acid (TfOH) or dimethyl(methylthio)sulfonium triflate (DMTST). The protected 2-deoxystreptamine was prepared chemoenzymaticly (Alper, P. B.; Hendrix, M.; Sears, P.; Wong, C.-H. J. Am. Chem. Soc. 1998, 120, 1965). The resulting pseudodisaccharide core 6 is purified and the α/β anomers are separated by silica gel column chromatography. In the most common case the hydroxyl and amine protecting groups were benzyl and azido, respectively. They were generally deprotected by basic hydrolysis of the acetates followed by hydrogenolysis using 1 atm. H₂ over 20% Pd(OH)₂/C to afford pseudodisaccharide 7; however, the deprotection conditions are not limited to these conditions. Alternatively, the acetate protecting groups of pseudodisaccharide 6 are removed by alkaline hydrolysis followed by selective glycosylation of the 6-O-position with suitably protected glycosyl donors (IIa) (IIa) (IVa) and (Va) or disaccharide donors (IIa-1) (IIa-1) (IVa-1) and (Va-1) to afford pseudotriisaccharide 8. In the most common case the hydroxyl and amine protecting groups were benzyl and azido, respectively. They were generally deprotected by hydrogenolysis using 1 atm. H₂ over 20% Pd(OH)₂/C C to afford pseudotriisaccharide 9; however, the deprotection conditions are not limited to these conditions. In cases were the glycosyl donor may have a unit of unsaturation hydrogenolysis was not employed. In cases such as these azides could be deprotected using trimethylphospine and hydroxyl protecting groups such as acetate could be removed by mild alkyline hydrolysis. The primary amines or hydroxyls of Ring I and III in structures 7 and 9 could be modified with amino acids, guanidinylating or amidinylating agents, isonitriles etc.

In the method of Scheme 6, glycosylation at the 4-position of of a suitably protected 2-DOS 5 using the method of programmable one-pot synthesis provides a pseudotrissacharide 7 as shown in Example 8. Two gycosyl donors are selected from (IIa) (IIIa) (IVa) or (Va). One glycosyl must have no reactive hydroxyls and must be easily activated. The second glycosyl donor must have one free hydroxyl and be approximately one tenth as reactive as the gylcosyl donor with no free hydroxyls.

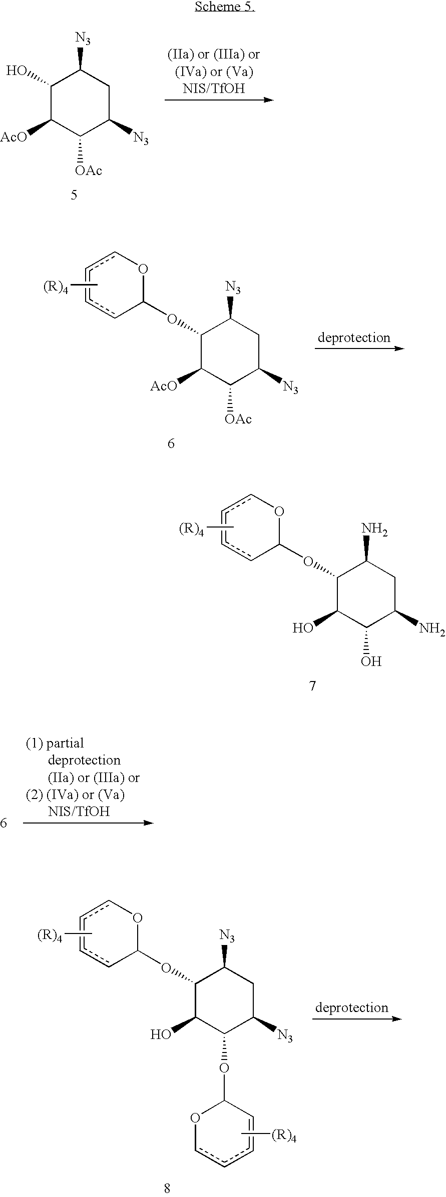

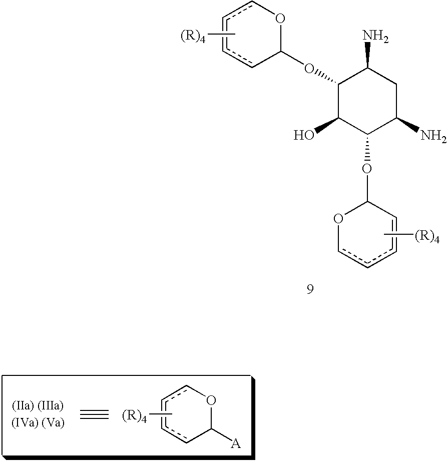

wherein: (R)₄ is described as in FIG. 4.

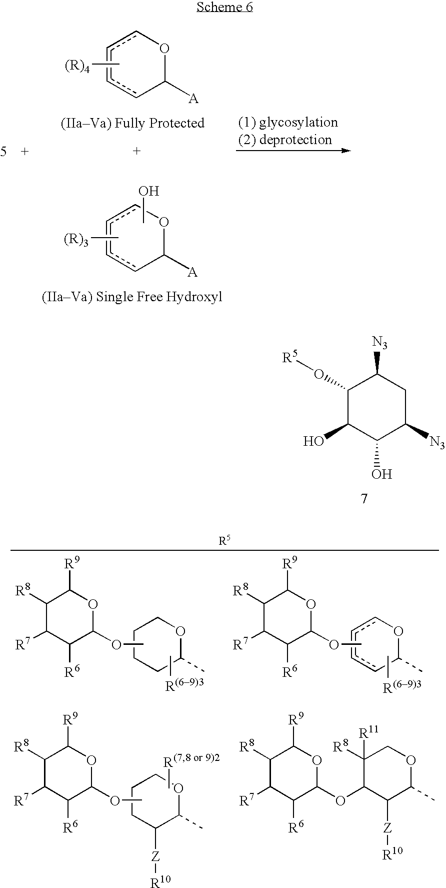

wherein: R⁶, R⁷, R⁸, R⁹ are as described in the text.

In the method of Scheme 7, commercially available 4,6-substituted AMGs, such as, tobramycin, bekanamycin, gentamicins, arbekacin, netilmicin and dibekacin are protected as the azides and a tosylate is selectively introduced using tosylchloride in pyridine to provide 8. As shown in Example 9 the tosylate can be displaced with a primary or secondary alkylamine or arlylalkyamine at the indicated temperature. Deprotection was effected by treatment of the perazido intermediate with trimethylphosphine-water to afford 6″-N-substituted aminoglycosides 9.

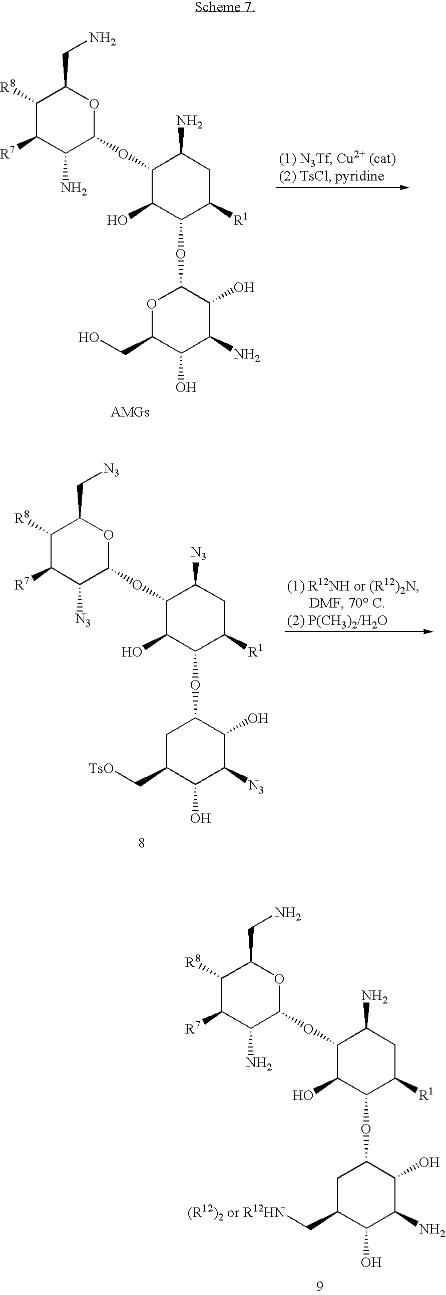

wherein: R¹, R⁷, R⁸, R¹² are as described in the text.

In the method of Scheme 8, commercially available 4,6-substituted AMGs, such as, tobramycin, bekanamycin, gentamicins, arbekacin, netilmicin and dibekacin are protected as the azides and a substituted carbonate can be introduced at the 6″-O-position using an isonitrile reagent to provide 10. Deprotection can be effected by treatment of the perazido intermediate with trimethylphosphine-water to afford 6″-O-substituted aminoglycosides 11

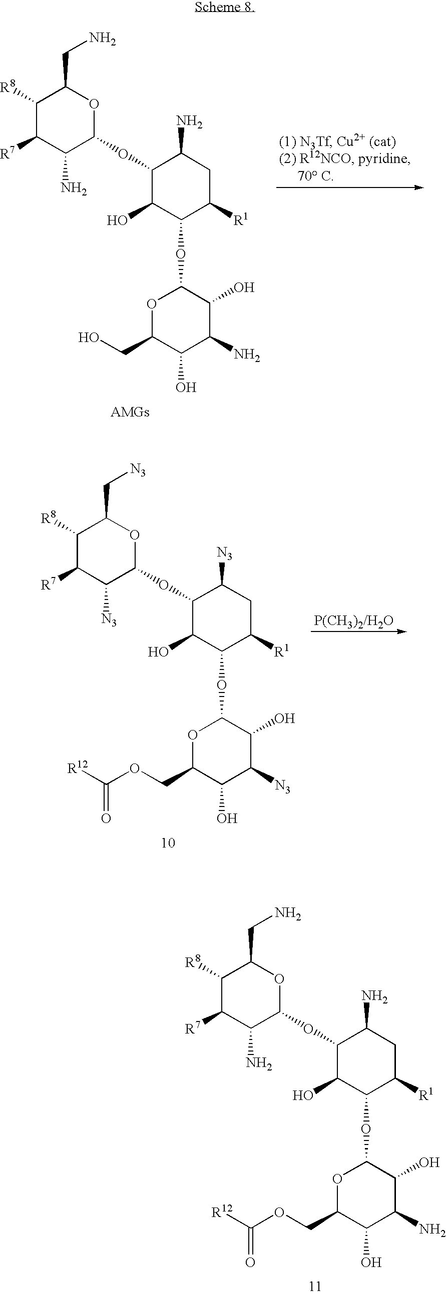

In the method of Scheme 9 the compounds related to structures (I) can be prepared from novel AMGs pseudodisaccharides 1 by alkylation of 1 with an R or S glycidyl tosylate to provide 12 as shown in Example 10. The epoxide is opened with any diamine. The resulting product is purified by silica gel column chromatography. The azido and benzyl groups were generally deprotected by hydrogenolysis using 1 atm. H₂ over Pd(OH)₂/C to afford 13a and 13b as shown in

### Example 11

However, the deprotection conditions are not limited to these conditions. The chemistry is identical when applied to the introduction of X¹—Y¹—R⁴.

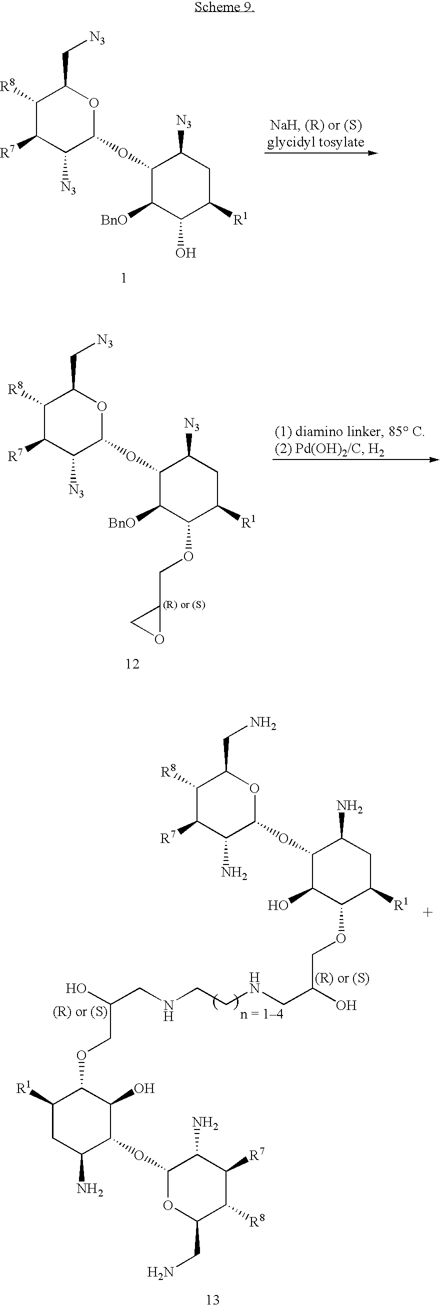

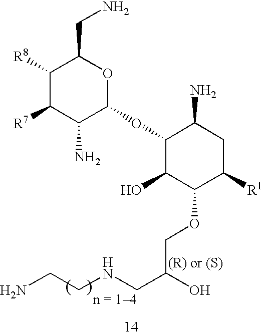

In the method of Scheme 10 the compounds related to structures (I) can be prepared from novel AMGs pseudodisaccharides 1 by alkylation of 1 with an allylhalide to provide 14, as shown in Example 12. The alkene is oxidized to the primary alcohol 15 as shown in Example 13 and converted to the tosylate 16 as shown in Example 14. The tosylate can then be displaced with any diamine. The azido and benzyl groups were generally deprotected by hydrogenolysis using 1 atm. H₂ over Pd(OH)₂/C to afford 17a and 17b as shown in Example 15. However, the deprotection conditions are not limited to these conditions. The chemistry is identical when applied to the introduction of X¹—Y¹—R⁴.

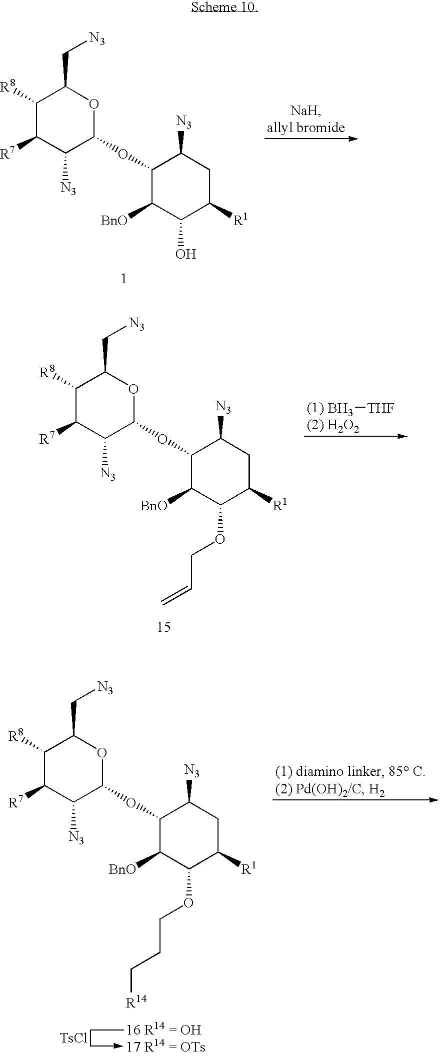

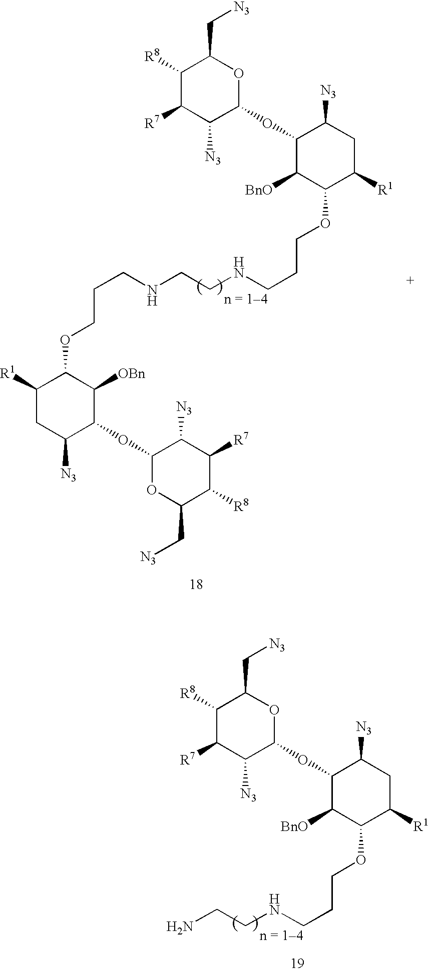

In the method of Scheme 11, intermediates required for the synthesis of compounds related to structures (I) when applied to the introduction of X¹—Y¹—R⁴ are described. Compound 20 or a related structure is protected by selective protection of the 6-hydroyl using dibutyltin oxide and benzyloxymethylchloride to produce structures 21 as shown in Example 19. The method is used to introduce of X¹—Y¹—R⁴. The methods of Scheme 9 and 10 were applied to pseudodisaccharide 21.

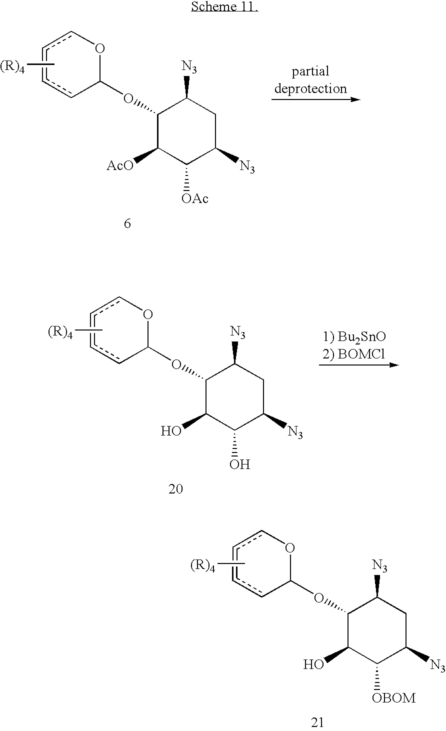

In the method of Scheme 12, intermediates required for the synthesis of compounds related to structures (I) when applied to the introduction of X¹—Y¹—R⁴ are described. Commercially available 4,6-substituted AMGs, such as, tobramycin, bekanamycin, gentamicins, arbekacin, netilmicin and dibekacin are chemically derivatized to provide the disaccharide cores 1 as previously described. The method involves reactions of present AMGs with triflic azide in the presence of copper (II) or zinc (II) catalyst followed by hydrolysis to provide the protected pseudodisaccharide cores 22 of the present invention as shown in Example 16. The ease of this process to synthesize advanced aminoglycoside intermediates with a free hydroxyl at the 4′,5,6-position is noteworthy. The overall yield over three steps can be as high as 80%. Compound 22 or a related structure is protected by selective protection of the 5 and 6-hydroyl using 1,1-dimethoxy cyclohexane ketal to afford 23 as shown in Example 17. The remaining 4′-hydroyl is protected using Na and benzylbromide to afford 24. The ketal is then removed using acid and a protic solvent to afford 25 as shown in Example 18. Compound 25 or a related structure is protected by selective protection of the 6-hydroyl using dibutyltin oxide and benzyloxymethylchloride using the method of Scheme 11 to introduce of X¹—Y¹—R⁴.

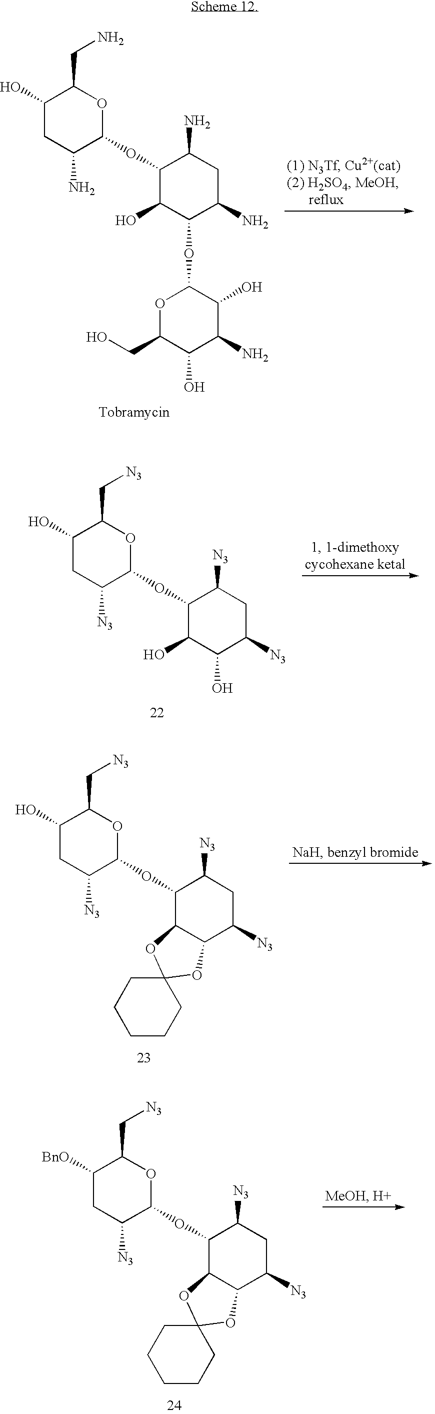

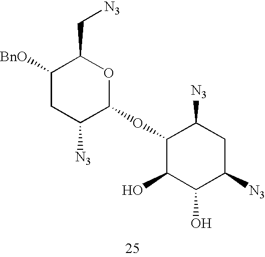

In the method of Scheme 13 the compounds related to structures (I) can be prepared from novel AMGs pseudodisaccharides by alkylation of of 21 with an R or S glycidyl tosylate to provide 26 as shown in Example 20. The epoxide is opened with any diamine. The resulting product is purified by silica gel column chromatography. The azido and benzyl groups were generally deprotected by hydrogenolysis using 1 atm. H₂ over Pd(OH)₂/C to afford 27 and 28 as shown in Example 21. However, the deprotection conditions are not limited to these conditions. The chemistry is identical when applied to the introduction of X¹—Y¹—R⁴.

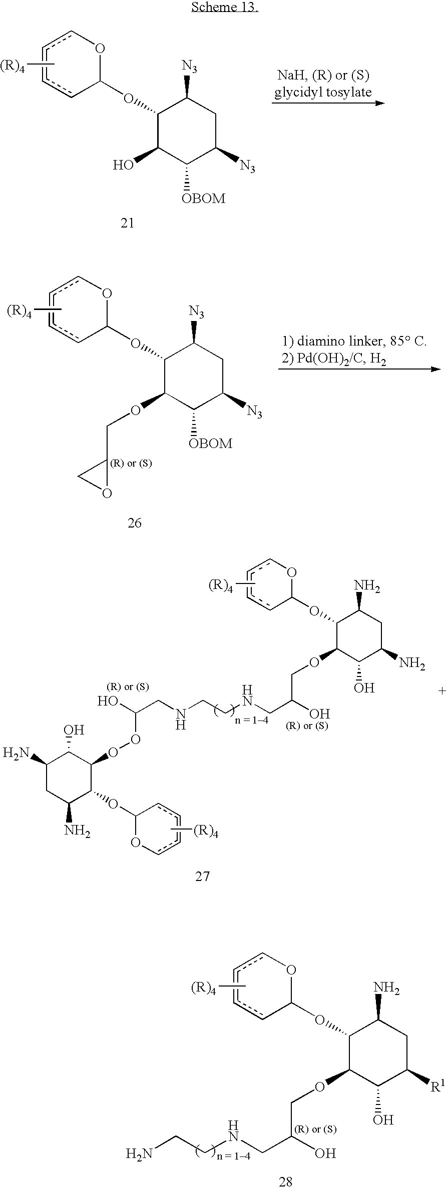

wherein: (R)₄ is described as in FIG. 4.

As can be appreciated from the disclosure above, the present invention has a wide variety of applications. Accordingly, the following examples are offered by way of illustration, not by way of limitation.

### EXAMPLES

### Example 1

### 5,4′-O-dibenzyl-perazidonebramine (1a)

The general procedure for the preparation of 5,4′-O-dibenzyl-perazidonebramine 1a. Tobramycin was treated with triflic azide in the presence of copper (II) or zinc (II) catalyst followed by benzylation with benzyl bromide. The benzylated intermediated is chemically derivatized by acidic methanolysis, to provide the pseudodisaccharide core 1a of the present invention.

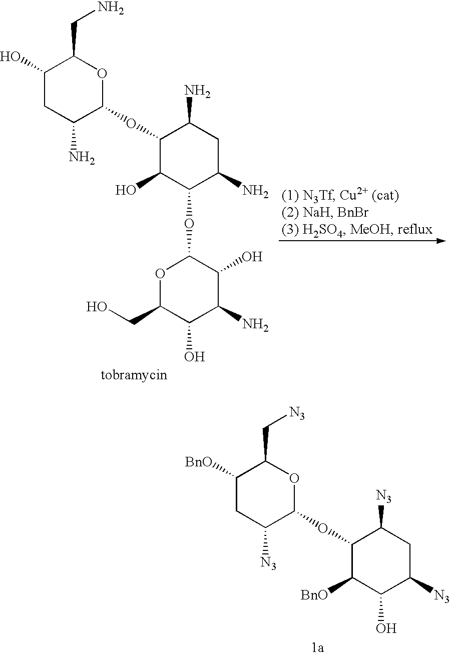

Part A: Preparation of perazidotobramyicn: Tobramycin pentasulfate (40 g, 56.1 mmol) was dissolved in 500 mL of water. A catalytic amount of CuSO₄.5H₂O (600 mg) (or zinc (II) catalyst) was added followed by 80 mL of triethylamine (16% the volume of H₂O). The mixture was diluted with 800 mL of MeOH (1.6 times the volume of water added) and cooled in an ice bath. Freshly prepared trifluormethane sulfonyl azide (561 mmol, 10 eq.) in dichloromethane was slowly added to this mixture. The reaction was allowed to warm to room temperature and stirred 16 h. The solution was concentrated to a volume consistent with that of the original amount of water added. The product was extracted four 300 mL-portions of ethyl acetate. The combined organics were washed 3 times with 1 M NaOH and 3 times with saturated ammonium chloride and the organic layer dried (Na₂SO4), filtered, and concentrated to afford the product as a hygroscopic foam: 33.0 (99.5%); silica gel TLC R_{f} 0.19 (1:2 hexanes-acetone); ¹H NMR (400 MHz, acetone-d₆) δ 1.66-1.78 (m, 1H), 2.02-2.09 (m, 2H), 2.20 (dt, 1H, J=11.5, 4.5 Hz), 2.51 (dt, 1H, J=12.5, 4 Hz), 3.04 (1H, br-s,), 3.25 (dt, 1H, J=12.3, 4.1 Hz), 3.38-3.90 (m, 10H), 3.97-4.03 (m, 1H), 4.09-4.15 (m, 1H), 4.41 (d, 1H, J=8 Hz), 4.52 (br-s, 1H), 4.78 (br-s, 1H), 4.87 (d, 1H, J=4 Hz), 5.22 (d, 1H, J=3 Hz), 5.64 (d, 1H, J=3.5 Hz); ¹³C NMR (100 MHz, acetone-d₆) δ 32.2, 32.7, 52.2, 57.3, 60.4, 60.6, 62.0, 66.2, 68.5, 69.8, 72.0, 73.8, 73.9, 76.4, 79.9, 84.4, 98.0, 99.5; mass spectrum (ESI), m/z 620.1 (M+Na)⁺ (C₁₈H₂₇N₁₅NaOg requires 620.2).

Part B: Preparation of perbenzyl-perazidotobramyicn: Preparation of perbenzyl-perazidotobramyicn: Perazidotobramycin (12.20 g, 20.4 mmol) was dissolved in 200 mL of dry DMF and cooled to 0° C. and purged with N₂. To the solution was added 12.25 g of 60% sodium hydride (306 mmol) in paraffin over a 30-45 minute period. The resulting mixture was allowed to stir for 30 to 45 minutes at 0° C. To the solution was added 26.71 mL of benzyl bromide (225 mmol) in a dropwise fashion over a 30-minutes. The reaction was allowed to warm to room temperature and stir for 2 hours. The completed reaction mixture was quenched with 400 mL of saturated ammonium chloride and extracted with three 300 mL-portions of diethyl ether. The combined organics were washed with water and brine. The organics were dried (Na₂SO₄), filtered and concentrated. The product was purified by flash column chromatography on silica gel (110 g). Elution with (9:1 hexanes-ethyl acetate) afforded the product as a light yellow oil: 16.5 g (77% yield); TLC R_{f} 0.47 (4:1 hexanes-ethyl acetate); ¹H NMR (400 MHz, CDCl₃) δ 1.63 (q, 1H, J=12.5 Hz), 2.01 (q, 1H, J=11.5 Hz,), 2.35 (qt, 2H, J=13, 4.5 Hz), 3.00 (dt, 1H, J=12.5, 4 Hz), 3.10 (dd, 1H, J=11, 2.5 Hz), 3.26 (dd, 1H, J=11, 1.5 Hz), 3.32-3.82 (m, 11H), 4.23 (t, 2H, J=11.5 Hz,), 4.46 (dd, 2H, J=12, 6 Hz), 4.58-4.83 (m, 6H), 4.91 (m, 2H), 5.5 (d, 1H, J=3.5 Hz), 5.64 (d, 1H, J=3.5 Hz), 6.94-7.44 (m, 25H); ¹³C NMR (100 MHz, CDCl₃) δ 27.8, 32.0, 56.2, 59.4, 60.2, 64.0, 65.4, 67.7, 70.1, 70.8, 71.0, 71.8, 73.1, 73.5, 74.5, 74.9, 75.9, 77.1, 77.4, 77.8, 83.3, 95.8, 96.3, 126.2, 127.2, 127.5, 127.8, 127.8, 127.9, 128.1, 128.1, 128.1, 128.2, 128.3, 128.5, 128.6, 137.3, 137.4, 137.5, 137.6, 137.9; mass spectrum (ESI), m/z 1070.4 (M+Na)⁺ (C₅₃H₅₇N₁₅NaOg requires 1070.4).

Part C: Preparation of 5,4′-O-dibenzyl-perazidonebramine: Perbenzyl-perazidotobramyicn (16.0 g, 15.3 mmol) was dissolved in 600 mL of methanol. To the solution was added 25 mL of concentrated sulfuric acid to make a 1.5 N methanolic solution. The reaction mixture was heated to gentle reflux for approximately 40 h, followed by careful TLC monitoring. After the starting material was consumed, the reaction mixture was cooled to room temperature and neutralized with 79 g of sodium bicarbonate (solid). The solution was concentrated to dryness, taken up in 800 mL of ethyl acetate. The solid was extracted with three 400 mL-portions of ethyl acetate. The combined organics were dried (Na₂SO₄) filtered, concentrated, and purified by flash column chromatography on silica (110 g). Elution with a linear gradient of (2%-10% acetone in hexane) afforded the product as a colorless oil: 4.82 g (65%); TLC R_{f} 0.37 (4:1 hexanes-ethyl acetate); ¹H NMR (400 MHz, CDCl₃) δ 1.55 (m, 3H), 2.1 (m, 1H), 2.31 (m, 1H), 2.40 (m, 1H), 3.1 (m, 1H), 3.48 (m, 6H), 3.62 (m, 1H), 4.22 (m, 1H), 4.58 (m, 2H), 4.90 (m, 1H), 5.52 (d, 1H, J=3.5 Hz), 7.33 (m, 10H); ¹³C NMR (100 MHz, CDCl₃) δ 28.19, 32.29, 51.55, 56.58, 60.00, 60.44, 71.31, 71.36, 72.37, 75.69, 77.41, 77.63, 85.29, 97.18, 128.14, 128.29, 128.45, 128.96, 129.09, 137.9, 138.29; mass spectrum (ESI), m/z 613.2 (M+Na)⁺ (C₂₆H₃₀N₁₂NaO₅ requires 613.2).

### Example 2a

### 4-(2,6-diamino-2,3,6-dideoxy-α-D-glucopyranosyl)-6-(2-amino-2-deoxy-1-α-D-glucopyranosyl)-2-deoxystreptamine (3a)

Part A: General procedure for the gycosylation between acceptor 1a and donors (IIb). To a dry flask containing flame-dried molecular sieves (500 mg) was added acceptor 1a (148.5 mg, 0.251 mmol) (1.0 equiv), corresponding donor (IIb) (1.5 equiv), and NIS (1.6 equiv) at room temperature under nitrogen. After cooling the flask to −78° C., anhydrous CH₂Cl₂ was added via syringe, and the mixture was stirred at −78° C. for 20 min. Then 1.0 M TfOH (0.15 to 0.3 equiv) was added at −78° C., and the reaction was stirred at −78° C. to −20° C. for 1 h and monitored by LTC. After finished, the reaction was quenched with solid NaHSO₃, NaHCO₃, and a few drops of H₂O. The mixture was stirred until colorless, then diluted with CH₂Cl₂, filtered, washed with NaHCO₃ and brine, dried over Na₂SO₄, and concentrated. The residue was purified by flash chromatography using Biotage FLASH12i using 2% to 15% EtOAc in hexane as eluent. (α,α)-isomer (162 mg, 61.5% yield) and ((α,β)-isomer (54 mg, 20.5% yield) as white solids. NMR data of. (α,α)-isomer: ¹H-NMR (CDCl₃): δ 1.06 (q, J=10.3 Hz, 1H), 2.02 (q, J=12.6 Hz, 1H), 2.32-2.42 (m, 2H), 3.03 (dt, J=12.6 Hz, 4.3 Hz, 1H), 3.13 (dd, J=11.0 Hz, 2.6 Hz, 1H), 3.26 (dd, J=11.0 Hz, 1.8 Hz, 1H), 3.42-3.84 (m, 13H), 4.16-4.24 (m, 1H), 4.30 (ABq, J=12.0 Hz, 2H), 4.48 (ABq, J=9.4 Hz, 2H), 4.63 (d, J=2.16 Hz, 1H), 4.66 (d, J=11.0 Hz, 1H), 4.79 (ABq, J=10.5 Hz, 2H), 4.93 (ABq, J=11.8 Hz, 2H), 5.45 (d, J=3.6 Hz, 1H), 5.67 (d, J=3.8 Hz, 1H), 7.15-7.38 (m, 25H). ¹³C-NMR (CDCl₃): 27.80, 32.44, 51.19, 56.24, 59.48, 61.02, 63.38, 67.64, 70.85, 70.91, 70.98, 71.82, 73.43, 74.62, 74.83, 75.37, 77.00, 77.88, 78.13, 80.06, 83.13, 96.37, 97.43, 126.17, 127.24, 127.44, 127.49, 127.67, 127.88, 127.94, 128.06, 128.13, 128.16, 128.29, 128.30, 128.45, 128.55, 137.37, 137.47, 137.75, 137.80, 138.21. ¹H NMR (CDCl₃) of (α,β)-isomer: δ 1.45 (q, J=12.7 Hz, 1H), 2.15 (q, J=11.4 Hz, 1H), 2.28-2.36 (m, 1H), 2.43-52 (m, 1H), 3.22 (dt, J=12.6 Hz, 4.3 Hz, 1H), 3.38-3.84 (m, 13H), 4.01-4.06 (m, 1H), 4.54-4.89 (m, 9H), 5.07 (d, J=10.2, 1H), 5.14 (d, J=10.2, 1H), 5.6 (d, J=3.6 Hz, 1H), 7.27-7.5 (m, 25H). ¹³C-NMR (CDCl₃): 27.81, 32.18, 51.29, 56.25, 58.21, 59.67, 66.65, 68.57, 70.94, 71.04, 71.98, 73.47, 74.63, 75.29, 75.56, 75.68, 78.04, 79.68, 83.16, 84.82, 96.64, 100.54, 127.46, 127.60, 127.76, 127.98, 128.03, 128.05, 128.13, 128.17, 128.47, 128.57, 128.60, 128.63, 137.62, 137.76, 137.84, 137.86, 138.54.

Part B: Deprotection procedure. To a 0.05 M THF solution of the product of Part A (1.0 equiv) was added 1 M THF solution of trimethylphosphine (0.2 n equiv, n=the number of the azido group in the corresponding trisaccharide) and H₂O (50−100 μL) under nitrogen. After stirring the reaction mixture at room temperature overnight, water (2-3 mL) was added, and the reaction mixture was stirred for another 0.5 h. Solvents were evaporated completely. The residue was dried under high-vacuum for 6 h, and then re-dissolved the residue in AcOH—H₂O (1:1, 0.02M), and the suspension was subjected to hydrogenolysis in the presence of 20% Pd(OH)₂ on carbon (0.2 m equiv, m=the number of benzyl group in the corresponding trisaccharide) under hydrogen balloon. After stirring for overnight, the reaction mixture was filtered and concentrated. The residue was purified by preparative-LCMS using YMC HPLC column (75×30 mm I.D., S-5 μm, 12 nm) with 10% to 30% MeCN (0.1% PFPA (v/v)) in water and a flow rate of 20 mL/min. The desired product 3a was collected at its corresponding masses and concentrated as PFPA salt. The product was further transferred into its free base by a small CG 50 (NH₄⁺) ion-exchange column using 0% to 6% NH₃H₂O in water to elute. The final product was collected according to TLC visualized by ninhydrin solution, concentrated and dried by lyophilization. NMR data of (α,α)-isomer: ¹H NMR (D₂O with a drop of DCI): δ 1.02-1.44 (m, 2H, CHH, CHH), 1.67-1.89 (m, 1H, CHH), 2.22-2.44 (m, 1H, CHH), 2.75-3.82 (m, 16H), 5.39 (d, J=3.9 Hz, 1H, anomeric proton), 5.54 (d, J=3.32, 1H, anomeric proton). ¹³C NMR (D₂O with a drop of DCI): 29.4, 30.3, 39.9, 47.9, 48.3, 49.5, 54.1, 60.3, 69.5, 69.4, 70.0, 70.1, 73.2, 73.9, 78.5, 82.8, 94.3, 96.9. Mass: 468.5 (M+1). NMR data of ((α,β)-isomer: ¹H-NMR (D₂O with a drop of DCI): δ 1.8-1.98 (m, 2H), 2.17 (dt, J=12.3, 4.4 Hz, 1H), 2.44 (dt, J=13.1, 4.2 Hz, 1H), 3.06 (dd, J=10.8, 8.3 Hz, 1H), 3.15 (dd, J=13.4, 7.1 Hz, 1H), 3.27-3.50 (m, 5H), 3.54-3.68 (m, 4H), 3.78-3.91 (m, 4H), 3.96-4.02 (m, 1H), 4.89 (d, J=7.9 Hz, 1H), 5.64 (d, 3.5 Hz, 1H). ¹³C-NMR (D₂O with a drop of DCI): δ 27.7, 29.1, 39.8, 47.8, 48.2, 48.4, 55.6, 60.2, 64.4, 69.3, 70.4, 71.5, 74.4, 76.4, 77.0, 78.7, 93.9, 99.0.

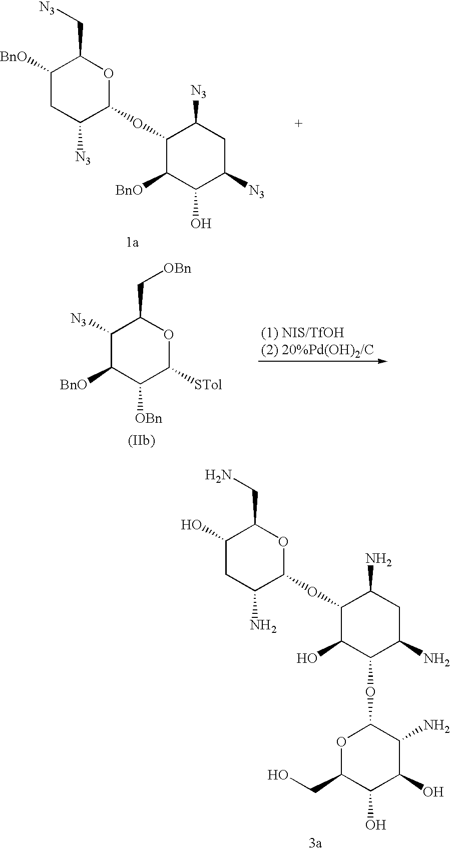

### Example 2b

### 4-(2,6-diamino-2,3,6-dideoxy-α-D-glucopyranosyl)-6-(4-amino-4-deoxy-1-α-D-glucopyranosyl)-2-deoxystreptamine (3b)

Using the procedure in Example 2a and substituting p-methylphenyl 2-azido-3,4,6-tri-O-benzyl-2-deoxy-1-thio β-D-glucopyranoside (IIc) for p-methylphenyl 4-azido-2,3,6-tri-O-benzyl-4-deoxy-1-thio β-D-glucopyranoside (IIb) gave title compound after deprotection with H₂ over 20% Pd(OH)₂/C.

### Example 2c

### 4-(2,6-diamino-2,3,6-dideoxy-α-D-glucopyranosyl)-6-(2-amino-2-deoxy-4-amino-4-deoxy-α-D-glucopyranosyl)-2-deoxystreptamine (3c)

Using the procedure in Example 2a and substituting p-methylphenyl 2,4-diazido-3,6-di-O-benzyl-2,4-dideoxy-1-thio β-D-glucopyranoside(IId) for p-methylphenyl 4-azido-2,3,6-tri-O-benzyl-4-deoxy-1-thio β-D-glucopyranoside (IIb) gave title compound after deprotection with H₂ over 20% Pd(OH)₂/C.

### Example 3

### 4-(2-amino-6-guanidinyl-2,3,6-dideoxy-α-D-glucopyranosyl)-6-(2-amino-2-deoxy-1-α-D-glucopyranosyl)-2-deoxystreptamine (4a)

Pseudodisaccharide core 3a and methyl-2 thio-2-imidazoline were dissolved in water. One equivalent of 1 N NaOH was added to the solution. The solution was warmed to 35° C. for 3 days to provide the selectively guanidinylated product.

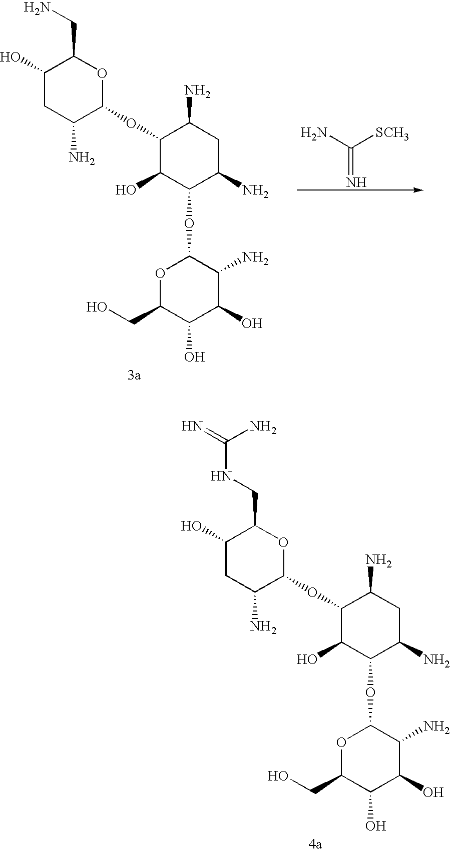

### Example 4

### 6,3′,4′,6′-O-tetrabenzyl-perazidoparomomine (1b)

Paromomycin was treated with triflic azide in the presence of copper (II) or zinc (II) catalyst followed by benzylation with benzyl bromide. The benzylated intermediated is chemically derivatized by acidic methanolysis, to provide the disaccharide cores 1b of the present invention.

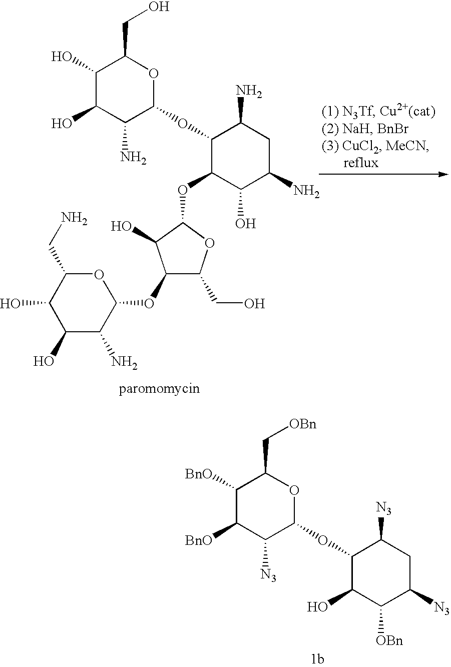

### Example 5

### Preparation of 1c

Butirosin was treated with triflic azide in the presence of copper (II) or zinc (II) catalyst followed by benzylation with benzyl bromide. The benzylated intermediated is chemically derivatized by refluxing with 2 equivalents of copper chloride dihydratre in dry acetonitrile to provide the pseudodisaccharide cores 1c of the present invention.

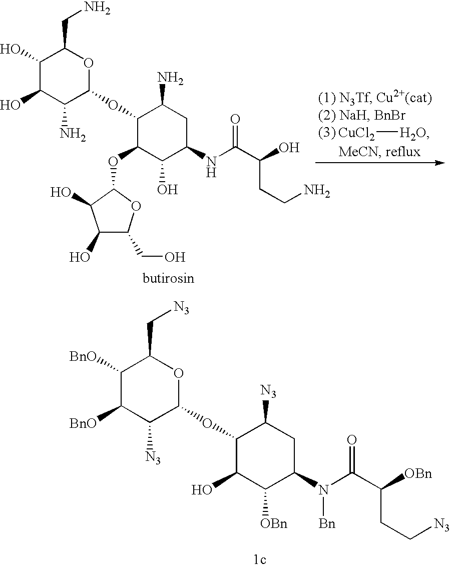

### Example 6

### 5-Amino-6-{3-amino-2-[4,6-diamino-3-(3-amino-6-aminomethyl-5-hydroxy-tetrahydro-pyran-2-yloxy)-2-hydroxy-cyclohexyloxy]-5-hydroxy-6-hydroxymethyl-tetrahydro-pyran-4-yloxy}-2-hydroxymethyl-tetrahydro-pyran-3,4-diol (3d)

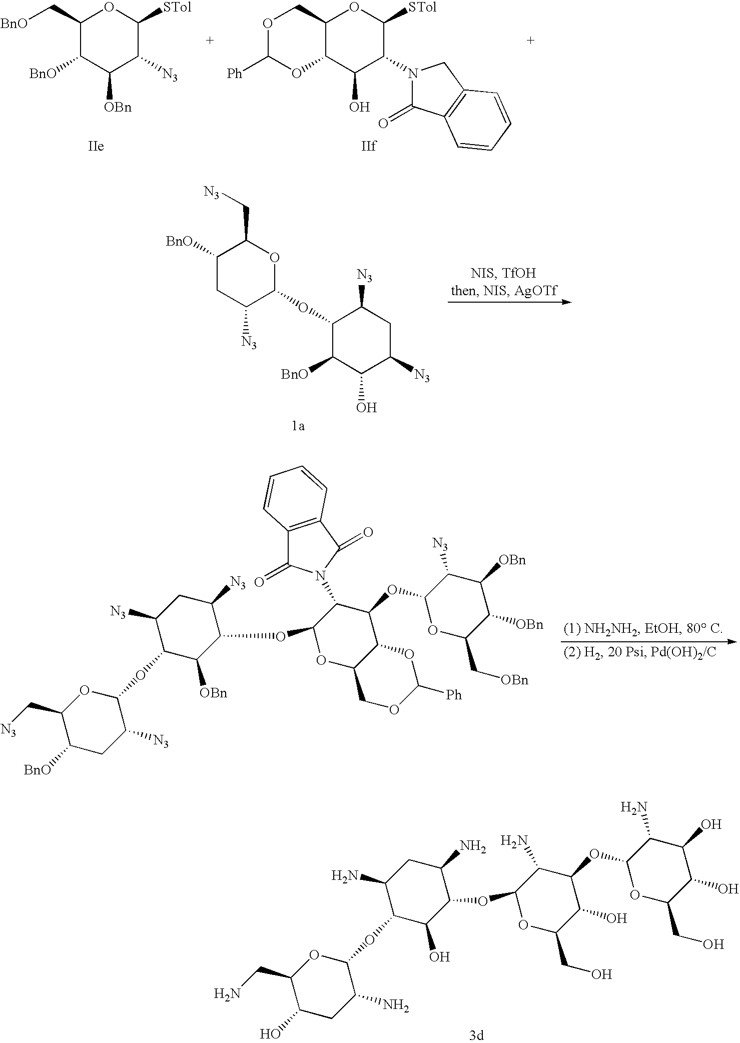

To a round flask containing thioglycoside IIe (1.5 eq.), thioglycoside IIf (1.25 eq.) and dried powered 4 Åmolecular sieves was added anhydrous dichloromethane. The solution was stirred at room temperature for 5 min, then cooled to −40° C., followed by addition of N-iodosuccinimide (NIS, 1.6 eq.) and 1 M TfOH ether solution (0.3 eq.). The reaction was stirred at −40° C. to rt until the reaction was completed as determined by TLC. The reaction mixture was re-cooled to −40° C., followed by addition of NIS and a dichloromethane solution of 1a (1.0 eq) was then added, followed by addition of NIS (1.3 eq.) and AgOTf (1.5 eq.). The reaction was stirred at −40° C. to rt until completion as determined by TLC (hexane: Ethyltate 5:1). Quenched the reaction by addition of NaHCO₃, Na₂SO₃ solution. Filtrate was dried over Na₂SO₄, purified by silica gel to afford the protected pseudotrisaccharide. The ethanol solution of the protected pseudotrisaccharide and NH₂NH₂H₂O (20 eq.) was heated to refluxed until reaction was finished. The solvent and excess of NH₂NH₂ was evaporated. The residue was taken up in acetic acid-water (2:1). To this solution was added Pd(OH)₂ (20%) (0.2 eq. for each protecting group). The reaction was subjected overnight to 15 psi of hydrogen. The reaction was monitored by mass spectrometry until completion. The reaction was filtrated, concentrated and purified by mass directed preparative HPLC to compound 3d as a colorless powder.

### Example 6b

### 5-Amino-6-13-amino-2-[4,6-diamino-3-(3-amino-6-aminomethyl-5-hydroxy-tetrahydro-pyran-2-yloxy)-2-hydroxy-cyclo hexyloxy]-5-hydroxy-6-hydroxymethyl-tetrahydro-pyran-4-yloxy}-2-hydroxymethyl-tetrahydro-pyran-3,4-diol (3d)

Aternatively, compound 3d can be obtained via step-wise glycosylation as outlined below. The disaccharide intermediate can be isolated, then resubjected to second glycosilation with pseudodisaccharide 1a to give compound 3d.

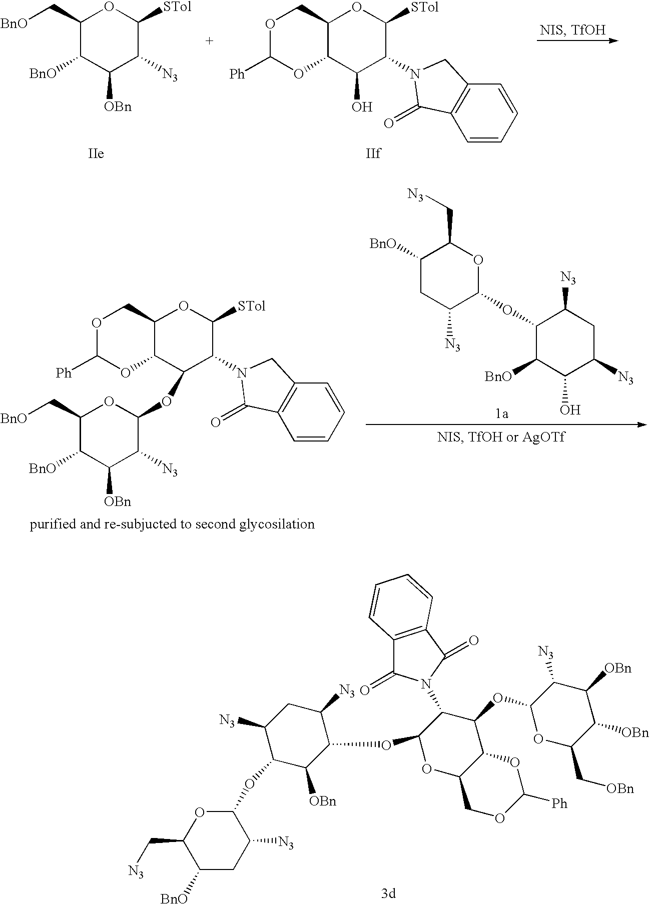

### Example 7

### Preparation of (3e)

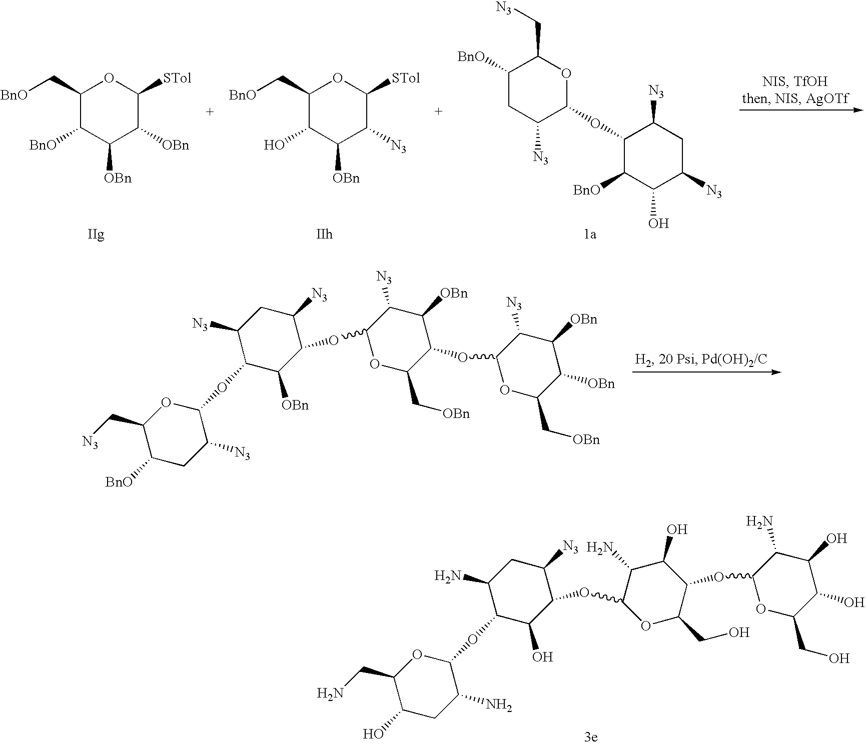

Using the procedure in example 6 and substituting thioglycoside IIg and IIh for IIe and IIf, respectively, for the glycosilation step gave the protected pseudotrisaccharide, which was deprotected using 15 psi hydrogen over Pd(OH)₂/C in acetic acid water (2:1) to provide compound 3e.

### Example 8

### Preparation of (7a)

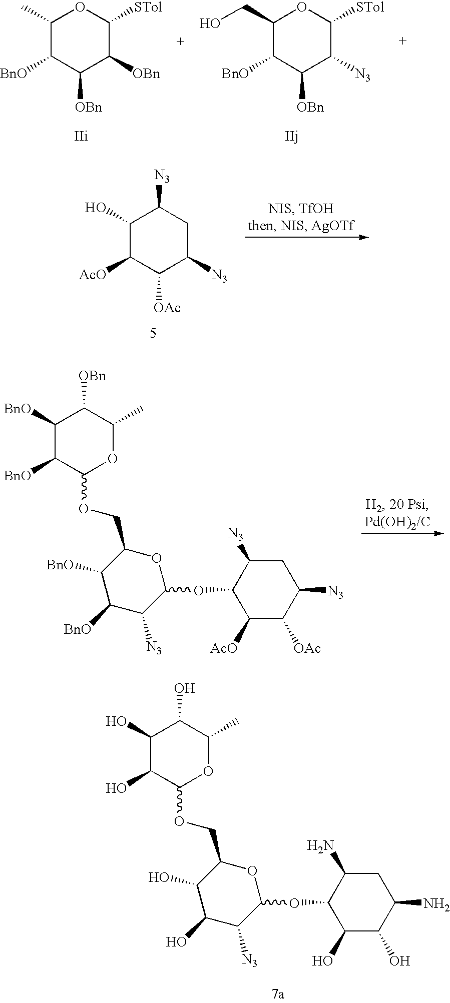

Part A: 100 μmol of IIi, 100 μmol of IIj, and 100 mg of activated molecular sieves placed in a 25 ml round bottom flask. Purged with nitrogen gas, and 1.1 ml of dry dichloromethane was added. The mixture was allowed to stir under nitrogen for 20 minutes. The reaction was cooled to −20° C. 32 mg (140 μmol, 1.4 eq.) of NIS was added and the mixture was allowed to stir for 10 minutes. 180 μl (36 μmol) of a 0.2 M solution of triflic acid in ether was added. The reaction was allowed to stir at −20° C., until the reaction was complete as determined by TLC (5:1, hexanes:ethyl acetate). 100 μmol of protected 2-deoxystreptamine 5 in 0.5 ml of dry dichloromethane were added to the reaction mixture. 40 μl of neat 2,6-di-tert-butylpyridine was added. The reaction was allowed to stir at −20° C. for 15 minutes. 32 mg (150 μmol, 1.5 eq.) of silver triflate were added. The reaction was allowed to stir at −20° C. until the reaction was complete as determined by TLC (5:1, hexanes:ethyl acetate). The completed reaction mixture was quenched with 1 ml of a 1:1 NaHCO₃:Na₂S₂O₃ solution, followed by filtration through silica gel and anhydrous Na₂SO₄, followed by layer separation, and concentration of the organics. The product was purified by column chromatography (8:1 hexanes:ethyl acetate) to yield 70 mg of pure product (65% yield). MS (m+H⁺): 1082.50.

Part B: 70 mg of pure tri-saccharide product from Part A was dissolved in 500 μl of methanol. This solution was treated with 100 μl of sodium methoxide (25% wt in methanol. The reaction was allowed to stir overnight at room temperature. The reaction was quenched with Dowex, filtered, and concentrated to give the de-acetylated product in quantitative yield (64 mg, 99%). MS (m+H⁺): 998.50. The crude product from the reaction was taken up in 1 ml of 2:1 acetic acid:water. To this was added 100 mg of Pd(OH)₂ (20%) catalyst was added. The reaction was subjected overnight to 15 psi of hydrogen. The reaction was monitored by mass spectrometry until complete. The reaction was filtered, concentrated and purified by mass directed preparative HPLC to give 12 mg of purified product (39% yield). MS (m+H⁺): 470.40.

### Example 9

### Preparation of (8a)

Tobramycin was treated with triflic azide in the presence of copper (II) or zinc (II) catalyst followed by selective tosylation to provide intermediate 8a. Displacement of the tosylate with a variety of primary and secondary amines (e.g., substituted piperidine) afforded a new series of amines. Reduction of the azides with P(CH₃)₃/H₂O or Pd(OH)₂/H₂ afforded the 6′-modified aminoglycosides 9a.

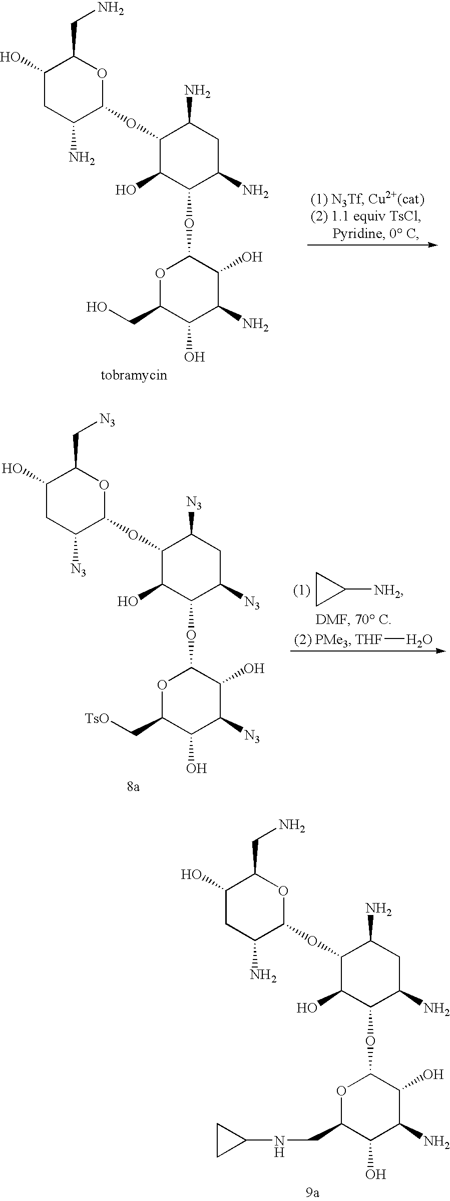

### Example 10

### 6-O-[(2R)-glycidyl]-perbenzyl-perazidonebramine (12a)

5,4′-O-dibenzyl-perazidonebramine 1a (2.78 g, 3.99 mmol) was dissolved in 30 mL dry DMF. The reaction mixture was purged with N₂ and cooled to 0° C. To the solution was added 240 mg of 60% NaH (5.99 mmol) in paraffin over a 30 minute period. The resulting solution was allowed to stir for 30-45 minutes at 0° C. To the solution was added 1.37 g of (2R)-(−)-glycidyl tosylate (5.99 mmol) over a 30-minute period. The reaction was allowed to warm to room temperature and stirred 2 hours. The reaction was quenched with saturated ammonium chloride, extracted with diethyl ether, dried (Na₂SO₄) filtered and concentrated. The product was purified by flash column chromatography (35 g). Elution with 8:1 hexanes-ethyl acetate afforded the product 12a as a colorless oil: 2.28 g (76% yield); silica gel TLC R_{f} 0.55 (4:1 hexanes-ethyl actate); ¹H NMR (400 MHz, CDCl₃) δ 1.42 (t, 1H, J=13 Hz); 1.99-2.11 (m, 1H); 2.26 (dt, 1H, J=13, 4.5 Hz); 2.37 (dt, 1H, J=11.5, 4.5 Hz); 2.50 (dd, 1H, J=5, 3 Hz); 2.78 (t, 1H, J=4.5 Hz); 3.07 (dt, 1H, J=13, 4 Hz); 3.17-3.22 (m, 1H); 3.22 (m, 1H); 3.25 (t, 1H, J=9 Hz); 3.33-3.70 (m, 5H); 4.04 (dd, 1H, J=10.5, 3 Hz); 4.17-4.24 (m, 1H); 4.47 (d, 1H, J=11.5 Hz); 4.65 (d, 1H, J=11.5 Hz); 4.84 (d, 1H, J=10.5 Hz); 5.01 (d, 1H, J=10.5 Hz); 5.50 (d, 1H, J=3.5 Hz); 7.33 (m, 10H); ¹³C NMR (100 MHz, CDCl₃) δ 28.16, 32.52, 40.87, 50.83, 51.58, 56.49, 59.95, 60.32, 71.27, 71.35, 72.36, 75.63, 75.66, 77.60, 84.67, 86.18, 97.17, 128.19, 128.25, 128.31, 128.47, 128.87, 128.96, 137.90, 138.2; mass spectrum (ESI), m/z 669.3 (M+Na)⁺ (C₂₉H₃₄N₁₂NaO₆ requires 669.3).

### Example 11

### Preparation of (13a) and (14a)

General Experamental Procedurure. Part A: Compounds 12a was treated with Y1 in the absence of solvent, or in the presence of an appropriate solvent such as ethanol, at temperatures ranging from 60° C. to 80° C.

Part B: The intermediate was treated with Y2 in an appropriate solvent, such as a 1:10 water-tetrahydrofuran, at ambient temperature.

Part C: Treatment with Y3 and gaseous hydrogen at 1 atmosphere in an appropriate solvent such as a 1:1 mixture of acetic acid and water at ambient temperature.

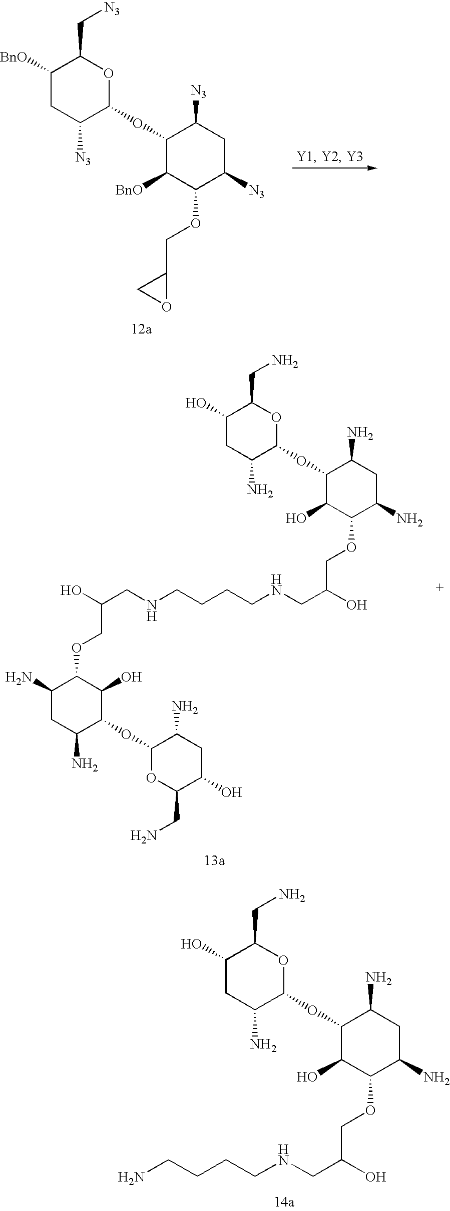

wherein: Y1 is a diamino linker such as 1,3-diaminobutane, 1,4-diaminobutane, 1,5-diaminobutane, or their N-alkylated, linear or branched, cyclic or aromatic analogues, their linear and branched homologues, and related polyamines or aminoethers.

wherein: Y2 is an azide reducing agent such as trimethylphosphine or triphenylphosphine.

wherein: Y3 is a catalyst such as wet palladium hydroxide on carbon or palladium-on-carbon.

Characterization of 13a; silica gel TLC R_{f} 0.68 (12:2:4:5 ammonium hydroxide-chloroform-ethanol-n-propanol); ¹H NMR (400 MHz, D₂O) δ 1.77 (m, 4H), 1.92 (ddd, 2H, J=13, 13, 13 Hz), 2.00 (ddd, 2H, J=9.5, 9.5, 9.5 Hz), 2.25 (ddd, 2H, J=4.5, 4.5, 13.5 Hz), 2.48 (ddd, 2H, J=12.5, 3.5, 3.5 Hz), 3.11 (m, 4H), 3.20-3.24 (m, 6H), 3.38-3.57 (m, 8H), 3.66 (m, 4H), 3.78-3.90 (m, 6H), 4.02 (m, 4H), 4.14 (m, 2H), 5.72 (s, 2H); ¹³C NMR (100 MHz, D₂O); ¹³C NMR (100 MHz, D₂O) δ 22.62, 28.01, 29.16, 39.80, 47.03, 47.77, 48.37, 49.02, 49.38, 64.43, 66.01, 70.12, 74.69, 75.38, 76.94, 81.61 and 94.02; mass spectrum (ESI+), m/z 813.5 (M+H)⁺, (C₃₄H₇₃N₁₀O₁₂ requires 813.5).

### Example 12

### Preparation of (15a)

Compound 1a is converted into compound 15a by treatment with Y4 and Y7, in an appropriate solvent such as N,N-dimethylformamide or tetrahydrofuran at temperatures ranging from ambient to 60° C.

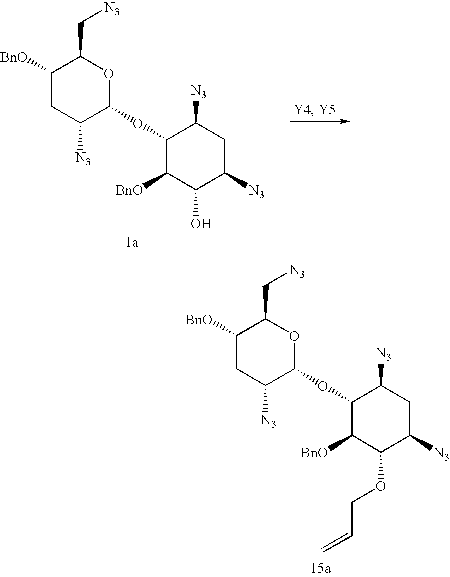

wherein: Y4 is a metal hydride base such as sodium hydride; wherein Y5 is an alkylating agent such as allyl bromide or allyl iodied.

### Example 13

### Preparation of (16a)

Compound 15a is converted into compound 16a by treatment with Y6 in an appropriate solvent such as dichloromethane, tetrahydrofuran or diethyl ether, followed by addition of a protic solvent such as methanol or ethanol, and addition of an aqueous solution of a base such as sodium hydroxide followed by Y7, at temperatures ranging from 0° C. to ambient.

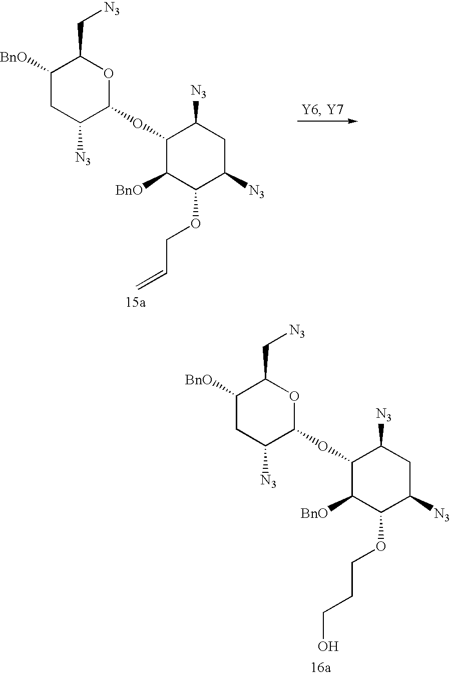

wherein: Y6 is a complex between borane and a Lewis base such as tetrahydrofuran or dimethylsulfide, or a monoalkylborane or dialkylborane such as texylborane, disiamylborane, diisopinocampheylborane or 9-borabicyclo[3.3.1]nonane; wherein Y7 is an oxidizing agent such as hydrogen peroxide.

### Example 14

### Preparation of (17a)

Reaction 6. Compound 16a is converted into compound 17a by treatment with Y8 in an appropriate solvent such as pyridine, a mixture of dichloromethane and pyridine, or a mixture of dichloromethane and triethylamine, at temperatures ranging from 0° C. to ambient.

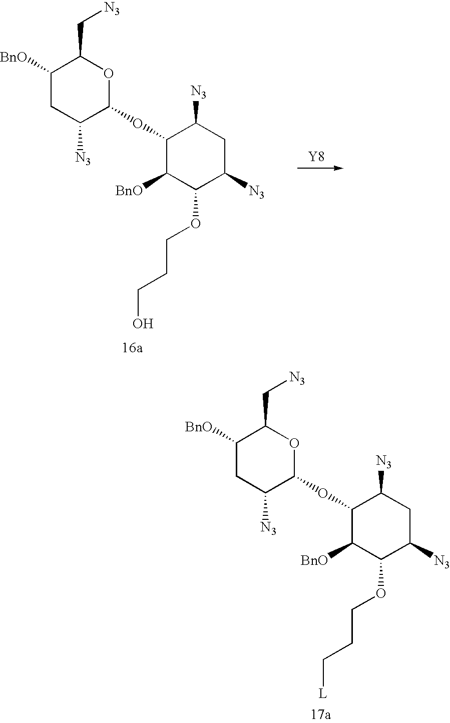

wherein: Y8 is a sulfonylating reagent selected from alkylsulfonyl halide, arylsulfonyl halide or trihaloalkylsulfonic anhydride, for example methanesulfonyl chloride, toluenesulfonyl chloride and trifluoromethanesulfonic anhydride;

wherein: L is a group able to undergo nucleophilic displacement, selected from alkylsulfonates, arylsulfonates or trihaloalkylsulfonates, for example methanesulfonate, toluenesulfonate and trifluoromethanesulfonate.

### Example 15

### Preparation of (18a) and (19a)

Step 1: Compound 17a was converted to compounds 18a and 19a by treatment with Y1 in the absence of solvent, or in the presence of an appropriate solvent such as dimethylformamide or dimethylsulfoxide, at temperatures ranging from 60° C. to 70° C.

Step 2: The intermediate was treated with Y2 in an appropriate solvent, such as a 1:10 water-tetrahydrofuran, at ambient temperature.

Step 3: Treatment with Y3 and gaseous hydrogen at 1 atmosphere in an appropriate solvent such as a 1:1 mixture of acetic acid and water at ambient temperature.

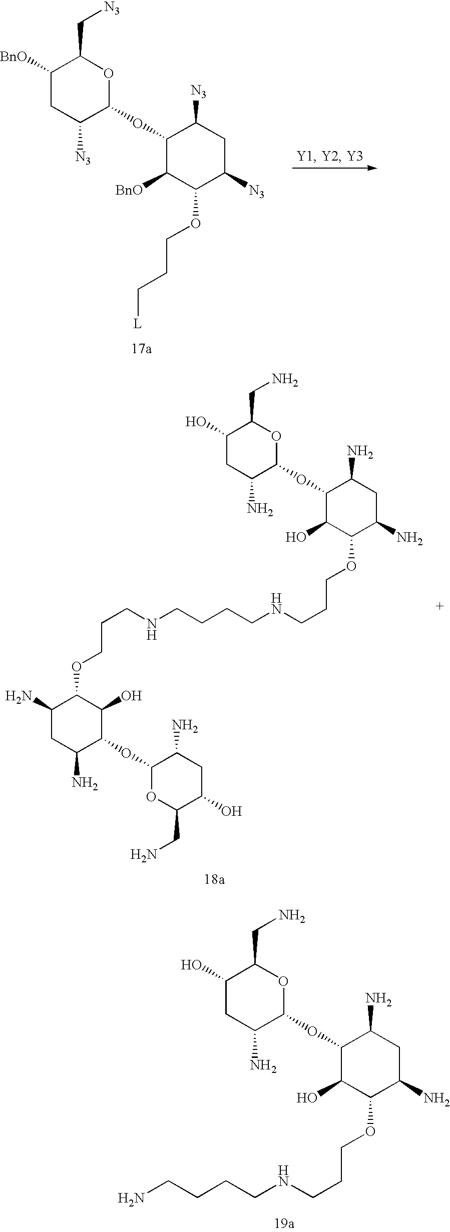

wherein: L is a group able to undergo nucleophilic displacement, selected from alkylsulfonates, arylsulfonates, trihaloalkylsulfonates or halides, for example methanesulfonate, toluenesulfonate, trifluoromethanesulfonate and iodide;

wherein: Y1 is a diamine as previously described.

wherein: Y2 is an azide reducing agent such as trimethylphosphine or triphenylphosphine.

wherein: Y3 is a catalyst such as wet palladium hydroxide on carbon or palladium-on-carbon.

### Example 16

### Preparation of (22)

Perazidotobramycin (from Part A, Example 1), (17.00 g, 28.5 mmol) was taken up in 600 mL of methanol and concentrated H₂SO₄ (25 mL) was slowly added to the solution. The mixture was heated to reflux and stirred 22 h. The solution was cooled to room temperature and neutralized with solid NaHCO₃. The solution was concentrated to dryness and the residue taken up in 100 mL of water. The solution was extracted with four 300 mL-portions of ethyl acetate, dried (Na₂SO₄), filtered and concentrated. The product was purified by flash column chromatography on silica (120 g). Elution with 7:3 hexanes-ethyl acetate afforded the product 22 as a colorless syrup: 7.59 g (65%); silica gel TLC R_{f} 0.10 (6:1 hexanes-ethyl acetate); ¹H NMR (400 MHz, acetone-d₆): δ 1.46-1.56 (m, 1H), 2.21 (dt, 1H, J=11, 4.5 Hz), 2.30-2.36 (m, 1H), 2.91 (s, 1H), 3.28 (dt, 1H, J=12.5, 4 Hz), 3.40-3.70 (m, 8H), 4.09-4.14 (m, 1H), 4.49 (d, 1H, J=5.5 Hz), 4.63 (s, 1H), 4.90 (s, 1H), 5.67 (d, 1H, J=3.5 Hz); ¹³C NMR (100 MHz, acetone-d₆): δ 32.22, 32.96, 52.23, 57.31, 60.76, 61.38, 66.19, 73.80, 77.56, 77.88, 79.86, 97.70; mass spectrum (ESI), m/z 393.0 (M−H₂O+H)+(C₁₂H₁₇N₁₂O₄ requires 393.1).

### Example 17

### Preparation of (23)

Perazidonebramine 22 (8.50 g, 20.70 mmol) was dissolved in 240 mL of a 1:1 toluene-acetonitrile solution. To the solution was added 5.97 g of 1,1-dimethoxycyclohexane (41.4 mmol) and a catalytic amount of p-toluenesulfonic acid (100 mg). The solution was placed on a rotary evaporator and allowed to stir at 50° C. under a reduced pressure of 400 mm Hg for 2 hours, followed by concentration to dryness. The crude mixture was taken up in diethyl ether and 20 mL of saturated sodium bicarbonate solution was added. The solution was extracted with three 50 mL-portions of diethyl ether. The organic were combined, dried (Na₂SO₄), filtered and concentrated. The product was purified by flash column chromatography on silica (120 g). Elution with 6:1 hexanes-ethyl acetate afforded the product 23 as a colorless oil: 8.84 g (87% yield); silica gel TLC R_{f} 0.40 (6:1 hexanes-ethyl acetate); ¹H NMR (400 MHz, CDCl₃) δ 1.34-1.72 (m, 12H), 2.24(dt, 1H, J=11.5, 4.5 Hz), 2.34(dt, 1H, J=13.5, 5 Hz), 3.22 (dt, 1H, J=12, 4 Hz), 3.40-3.60 (m, 5H), 3.63-3.73 (m, 2H), 3.85-3.92 (m, 2H), 5.50 (d, 1H, J=3.5 Hz,); ¹³C NMR (100 MHz CDCl₃): δ 23.68, 23.72, 24.86, 31.39, 33.82, 35.99, 36.24, 51.30, 56.05, 57.22, 61.13, 65.94, 72.35, 77.01, 79.25, 79.45, 95.28, 113.74; mass spectrum (ESI), m/z 476.0, 415.1, 371.1, 327.0, 283.0, 238.9 (M+H)+(C₁₈H₂₇N₁₂O₅ requires 491.2).

### Example 18

### Preparation of (24 and 25)

PART A: 4,5-O-cyclohexylidine perazidonebramine 23(1.2 g, 2.45 mmol) was taken up in 10 mL of dry DMF. The solution was cooled to 0 IC for 30 minutes followed by the addition of 60% NaH (117 mg, 2.94 mmol) in paraffin. The mixture was allowed to stir at 0 IC for 30 minutes. Benzyl bromide (502 mg, 2.94 mmol) was added dropwise at 0 IC. The mixture was allowed to warm up to room temperature after the addition of benzyl bromide was complete. The reaction was stirred 2 hours and quenched with 50 mL of aqueous ammonium chloride. The solution was extracted with 3 50 mL-portions of diethyl ether, dried (Na₂SO₄), filtered and concentrated. The product was purified by flash column chromatography on silica (35 g). Elution with 17:3 hexanes-ethyl acetate afforded the product 24 as a colorless oil: 1.19 g (84% yield); silica gel TLC R_{f} 0.80 (4:1 hexanes-ethyl acetate); ¹H NMR (400 MHz, CDCl₃) δ 1.26-1.72 (m, 11H), 1.96-2.08 (m, 1H), 2.22-2.37 (m, 2H), 3.10 (dt, 1H, J=13, 3.5 Hz,), 3.33-3.62 (m, 7H), 3.83 (t, 1H, J=9.5 Hz), 3.98-4.06 (m, 1H), 4.43 (d, 1H, J=11.5 Hz,), 4.63 (d, 1H, J=11.5 Hz), 5.44 (d, 1H, J=3 Hz), 7.2-7.4 (m, 5H); ¹³C (100 MHz, CDCl₃) δ 23.76, 24.94, 28.36, 33.53, 36.02, 36.27, 51.24, 55.75, 56.61, 60.97, 61.03, 70.78, 72.14, 72.18, 79.22, 79.50, 95.53, 95.57, 113.61, 127.72, 128.12, 128.46, 137.70.

PART B: 4′-O-benzyl-4,5-O-cyclohexylidine perazidonebramine 24 (1.19 g, 2.05 mmol) from PART A was dissolved in 100 mL of methanol. To the solution was added 1 mL of concentrated sulfuric acid. The solution was stirred at room temperature until complete. The solution was neutralized with saturated sodium bicarbonate, extracted with diethyl ether, dried (Na₂SO₄), filtered and concentrated. The product was purified by flash column chromatography on silica (35 g). Elution with a linear gradient of 10-40% ethyl acetate in hexanes afforded the product 25 as a colorless crystalline solid: 950 mg (92% yield); mp 80-81 IC; silica gel TLC R_{f} 0.20 (6:1 hexanes-ethyl acetate); ¹H NMR (400 MHz, CDCl₃) δ 1.46-1.58 (m, 1H), 1.99 (q, 1H, J=12 Hz), 2.32 (dt, 1H, J=13.5, 4 Hz), 2.41 (dt, 1H, J=11.5, 4.5 Hz), 3.30-3.60 (m, 10H), 4.08-4.14 (m, 1H), 4.50 (d, 1H, J=11.5 Hz), 4.67 (d, 1H, J=11.5 Hz), 5.20 (d, 1H, J=3.5 Hz), 7.29-7.41 (m, 5H); ¹H NMR (100 MHz, CDCl₃) δ 28.18, 32.01, 51.15, 57.52, 58.97, 59.19, 71.13, 71.16, 71.81, 75.52, 76.23, 82.07, 97.81, 127.88, 128.13, 128.59, 137.42; mass spectrum (ESI), m/z 523.1 (M+H)+(C₁₉H₂₄N₁₂NaO₅ requires 523.2

### Example 19

### Preparation of (21a)

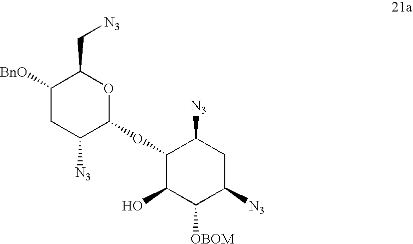

Preparation of 6-O-benzyloxymethoxy-4′-O-benzyl perazidonebramine (21 a): 4′-O-Benzyl perazidonebramine 24 (2.62 g, 5.23 mmol) was dissolved in toluene (50 mL) and added to a flask equipped with a Dean-Strark separator containing 3.43 g of dibutyl tin oxide (5.76 mmol). The solution was refluxed for 1 hour and cooled to room temperature. Benzyloxymethyl chloride (8.15 g, 52.3 mmol) and 2.07 g of tetrabutylammonium iodide (5.23 mmol) were added to the solution and stirred at room temperature for 6 hours. The reaction was quenched with sodium bicarbonate, washed with diethyl ether and evaporated to dryness. The product was purified by flash column chromatography on silica (35 g). Elution with 8:1 hexanes-ethyl acetate afforded the product 21a as a colorless oil: 1.50 g (46%); silica gel TLC R_{f} 0.55 (6:1 hexanes-ethyl acetate); ¹H NMR (400 MHz, CDCl₃) δ 1.42 (1H, ddd, J=12.4, 13.0 Hz), 2.00 (1H, ddd, J=11.9, 12.1 Hz), 2.22 (1H, ddd, J=4.3, 13.0 Hz), 2.33 (1H, ddd, J=4.4, 11.4 Hz), 3.16 (1H, ddd, J=3.8, 12.6 Hz), 3.62-3.24 (13H, m), 4.13 (1H, m), 4.34 (1H, d, J=2.0 Hz), 4.45 (2H, d, J=11.5 Hz), 4.63 (2H, d, J=11.4 Hz), 4.73 (1H, d, J=11.2 Hz), 4.81 (1H, d, J=7.0 Hz), 5.02 (1H, d, J=7.0 Hz), 5.47 (1H, d, J=3.4 Hz), 7.38-7.22 (10H, m);

¹³C NMR (150 MHz, CDCl₃) δ 11.30, 28.52, 32.46, 46.18, 51.62, 57.14, 59.39. 59.52, 60.02, 70.19, 71.00, 71.27, 71.34, 71.36, 72.48, 76.33, 80.61, 85.73, 96.72, 97.69, 127.42, 128.00, 128.26, 128.32, 128.49, 128.59, 128.32, 128.49, 128.63, 128.91, 128.95, 128.99, 129.06, 137.10, 137.98, 141.37; mass spectrum (ESI), m/z 643.2 (M+Na)⁺ (C₂₇H₃₂N₁₂NaO₆ requires 643.3).

### Example 20

### Preparation of (26a)

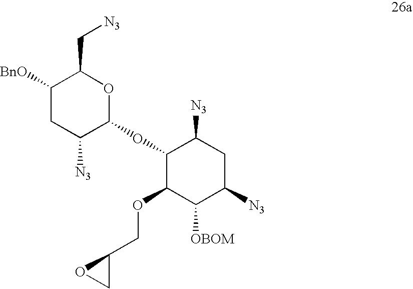

Preparation of 5-O-[(2R)-glycidyl]-6-O-benzyloxymethoxy-4′-O-benzyl perazidonebramine (26a): Crude 21a (1.00 g, 1.61 mmol) was taken up in 10 mL of DMF and treated with 644 mg of 60% sodium hydride in paraffin (16.1 mmol). To the solution was added 3.68 g of (2R)-(−)-glycidyl tosylate (16.1 mmol). The reaction was stirred 16 h and quenched with aqueous ammonium hydroxide. The solution was extracted with tree 100 mL-portions of diethyl ether. The organic layers were combined and dried (MgSO₄). The product was purified by flash column chromatography on silica gel (Biotage 40 M). Elution with 6:1 hexane-ethyl acetate afforded the product as a colorless glass: yield 200 mg; silica gel TLC R_{f} 0.57 (4:1 hexanes-ethyl acetate); ¹H NMR (400 MHz, CDCl₃) □ 1.45 (ddd, 1H, J=13 Hz), 1.99 (ddd, 1H, J=13 Hz), 2.27 (m, 1H), 2.32 (m, 1H), 2.52 (dd, 1H, J=2.5, 5 Hz), 2.67 (t, 1H, J=4.5 Hz), 3.09 (m, 2H), 3.27-3.51 (m, 9H), 3.70 (dd, 1H, J=6, 10.5 Hz), 4.00 (dd, 1H, J=2.5, 10.5 Hz), 4.15 (m, 1H), 4.41 (d, 1H, J=11.5 Hz), 4.60 (d, 1H, J=11.5 Hz), 4.75 (d, 1H, J=11.5 Hz), 4.88 (d, 1H, J=6.5 Hz), 4.96 (d, 1H, J=6.5 Hz), 5.44 (d, 1H, J=3.5 Hz), 7.20-7.31 (m, 10H); ¹³C NMR (100 MHz, CDCl₃) □ 28.23, 32.62, 44.50, 50.90, 51.54, 56.74, 59.81, 60.04, 60.20, 70.95, 71.34, 71.42, 72.44, 74.50, 77.52, 81.03, 85.421, 96.61, 97.19, 128.07, 128.16, 128.19, 128.27, 128.46, 128.86, 128.96, 137.94, 138.08; mass spectrum (ESI), m/z 700 (M+Na)⁺ (C₃₀H₃₆N₁₂NaO₇ requires 699).

### Example 21

### Preparation of (27a) and (28a)

General Experamental Procedurure. Part A: Compounds 26a was treated with Y1 in the absence of solvent, or in the presence of an appropriate solvent such as ethanol, at temperatures ranging from 60° C. to 80° C.

Part B: The intermediate was treated with Y2 in an appropriate solvent, such as a 1:10 water-tetrahydrofuran, at ambient temperature.

Part C: Treatment with Y3 and gaseous hydrogen at 1 atmosphere in an appropriate solvent such as a 1:1 mixture of acetic acid and water at ambient temperature.

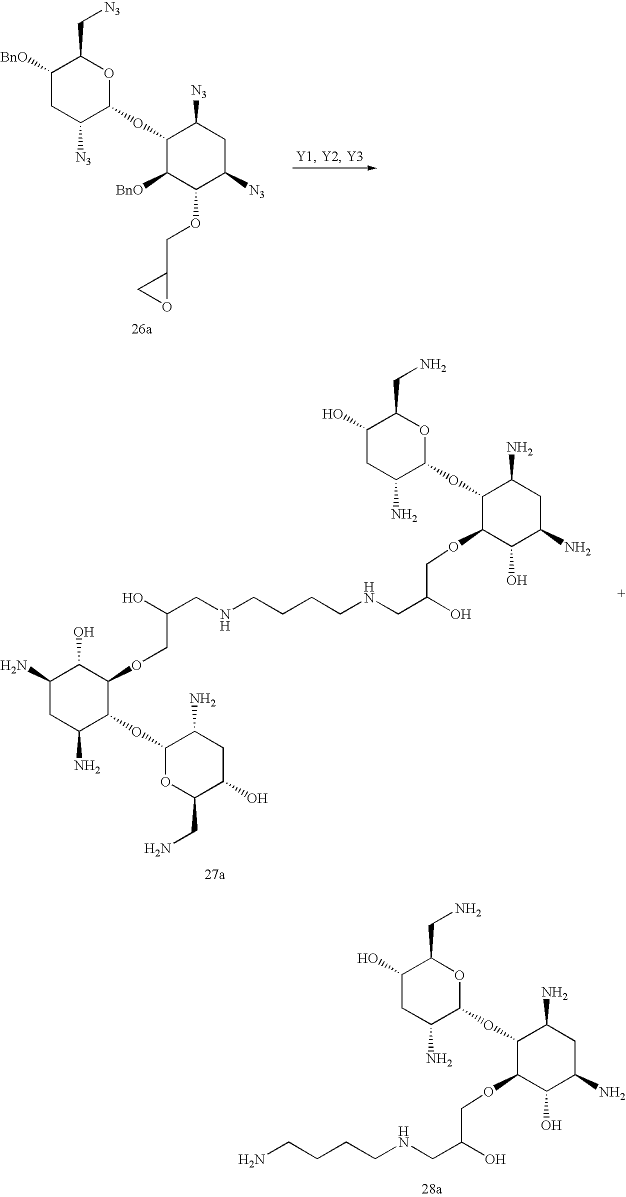

wherein: Y1 is a diamino linker such as 1,3-diaminobutane, 1,4-diaminobutane, 1,5-diaminobutane, or their N-alkylated, linear or branched, cyclic or aromatic analogues, their linear and branched homologues, and related polyamines or aminoethers.

wherein: Y2 is an azide reducing agent such as trimethylphosphine or triphenylphosphine.

wherein: Y3 is a catalyst such as wet palladium hydroxide on carbon or palladium-on-carbon.

Characterization of 27a; silica gel TLC R_{f} 0.68 (12:2:4:5 ammonium hydroxide-chloroform-ethanol-n-propanol); ¹H NMR (400 MHz, D₂O) δ 1.75 (m, 4H), 1.92 (ddd, 2H, J=13, 13, 13 Hz), 2.02 (ddd, 2H, J=9.5, 9.5, 9.5 Hz), 2.21 (ddd, 2H, J 4.5, 4.5, 13.5 Hz), 2.48 (ddd, 2H, J=12.5, 3.5, 3.5 Hz), 3.09 (m, 4H), 3.21-3.34 (m, 12H), 3.52 (m, 2H), 3.60 (dd, 2H, J=9, 9 Hz), 3.67-3.79 (m, 6H), 3.83 (m, 2H), 3.95 (m, 2H), 4.06 (m, 2H), 4.12 (m, 4H), 5.57 (d, 2H, J=3 Hz); ¹³C NMR (100 MHz, D₂O) δ 22.7, 27.9, 28.7, 39.3, 47.2, 47.4, 48.6, 49.7, 49.8, 50.3, 63.9, 65.9, 72.7, 73.9, 76.3, 82.8 and 93.5; mass spectrum (ESI+), m/z 814.4 (M+D)⁺, (C₃₄H₇₂DN₁₀O₁₂ requires 814.6).

### Example 22

In Vitro Antibacterial Activity Determination of Minimum Inhibitory Concentrations (MICs).

The assays are carried out in 96-well flat-bottom plates. The bacterial suspension from an overnight culture grown in appropriate medium is added to a solution of test compound in water. Final bacterial inoculums are approximately 10⁵-10⁶ CFU/well. The percent growth of bacteria in test wells relative to that observed for a well containing no compound is determined by measuring absorbance at 595 nm (A₅₉₅) after 24 h. The MIC (μg/mL) is determined as a range of single compound where the complete inhibition of growth is observed at the higher concentration and cells are viable at the lower concentrations. Tobramycin is used as an antibiotic-positive control in each screening assay for Pseudomonas aeruginosa, Acinetobacter species, Streptococci viridans, Proteus species, Hemophilus influenzae, Citrobacter species, Serratia marcescens, Staphylococcus aureus, Streptococcus pyogenes, Escherichia coli, Enterobacter species, Enterococcus faecalis, Enterococcus faecium Klebsiella pneumoniae and Providencia stuartii. Representative data is shown in Table 1.

TABLE 1
Selected MICs (μg/mL) of Synthetic Aminoglycosides (AMGs)
Against a Panel of Gram-Negative and Gram-Positive Microorganisms

| | | | | S. aureus | S. aureus | | |
|---|---|---|---|---|---|---|---|
| | P. aeruginosa | P. aeruginosa | P. aeruginosa | ATCC 29213 | ATCC 33591 | E. coli | E. faecalis |
| Compound | ATCC 27853 | ATCC 35151 | PAO-1 | (MSSA) | (MRSA) | ATCC 25922 | ATCC 29212 |
| 3a | 1 | 1 | 0.5 | 1 | >64 | 2 | 16 |
| 3b | 16 | 16 | 8 | 32 | >64 | 32 | >64 |
| 3c | 2 | 1 | 1 | 1 | >64 | 2 | 32 |
| 3d | 64 | 16 | 32 | 16 | >64 | 32 | >64 |
| 3e | 32 | 16 | 32 | 32 | >64 | 64 | >64 |
| 4a | >64 | >64 | 64 | 64 | >64 | 64 | >64 |
| 7a | >64 | >64 | >64 | >64 | >64 | >64 | >64 |
| 9a | 64 | 32 | 32 | 8 | 8 | 32 | >64 |
| 13a | 2 | 4 | 2 | 0.5 | 16 | 4 | 32 |
| 14a | 32 | 16 | 16 | 8 | >64 | 16 | >64 |
| 18a | 16 | 32 | 8 | 4 | 32 | 16 | >64 |
| 19a | >32 | >32 | >32 | >32 | >32 | >32 | >32 |
| 27a | 4 | 8 | 4 | 2 | 16 | 16 | 32 |
| Tobramycin | 0.5 | 0.5 | 0.25 | 0.5 | >64 | 1 | 16 |

It is to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. Compounds having the structure of (1), as well as pharmaceutically acceptable salts, prodrugs and solvates thereof: 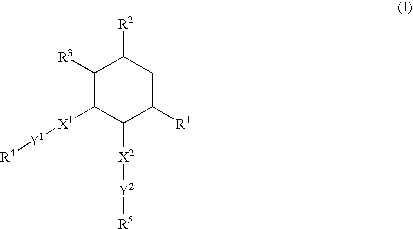
wherein,
R¹ and R² are independently amino, protected amino or modified amino,
X¹ and X² are independently O, S or NH,
Y¹ or Y² is a bond or a divalent linking group,
R³ is selected from the group consisting of the formula (II) or (III): 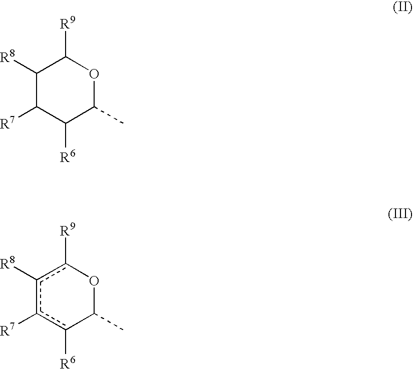
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1). 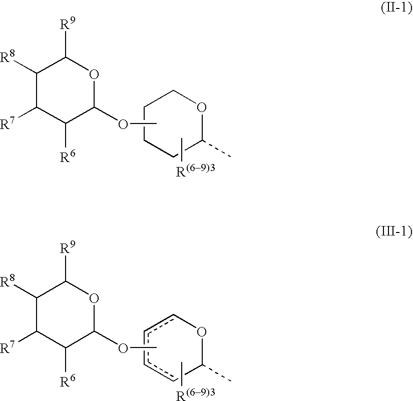
one of R⁴ and R⁵ is hydrogen, hydroxyl protecting or modified hydroxyl group when one of Y¹ or Y² is a bond and the other is selected from a group consisting of formula (II), (III), (IV), (V), (VI) or (VII): 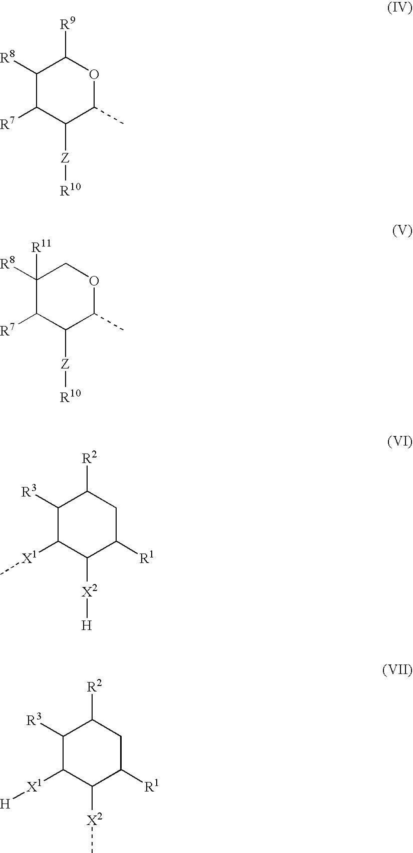
Z can be O, S or NH,
R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another monoe- or disaccharide (II), including disaccharides (IV-1) and (V-1) 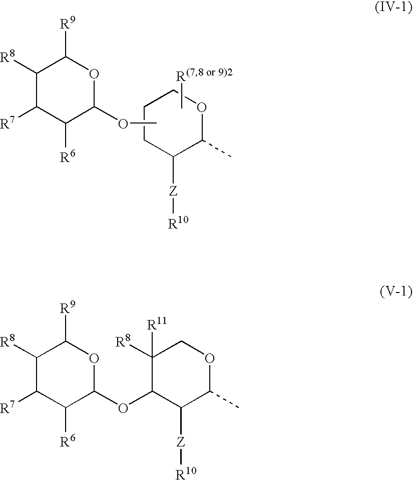
R¹⁰ can be hydrogen, an alkyl group, an amine protecting group, modified amino, hydroxyl protecting or modified hydroxyl group, and
R¹¹ can be a hydrogen, halogen or alkyl group.

2. A method for synthesizing compounds having the structure of (I), as well as pharmaceutically acceptable salts, prodrugs and solvates thereof: 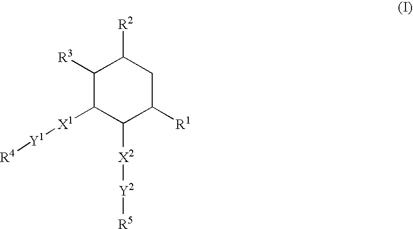
wherein,
R¹ and R² are independently amino, protected amino or modified amino,
X¹ and X² are independently O, S or NH,
Y¹ or Y² is a bond or a divalent linking group,
R³ is selected from the group consisting of the formula (II) or (III): 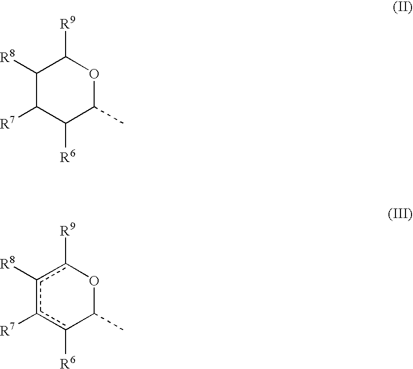
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1). 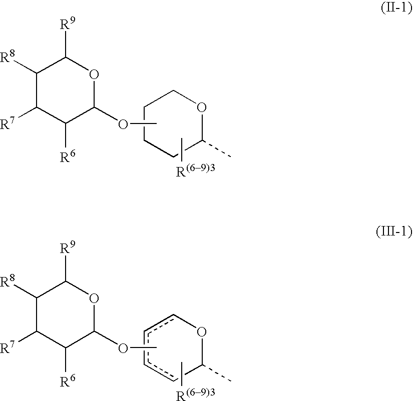
one of R⁴ and R⁵ is hydrogen, hydroxyl protecting or modified hydroxyl group when one of Y¹ or Y² is a bond and the other is selected from a group consisting of formula (II), (III), (IV), (V), (VI) or (VII): 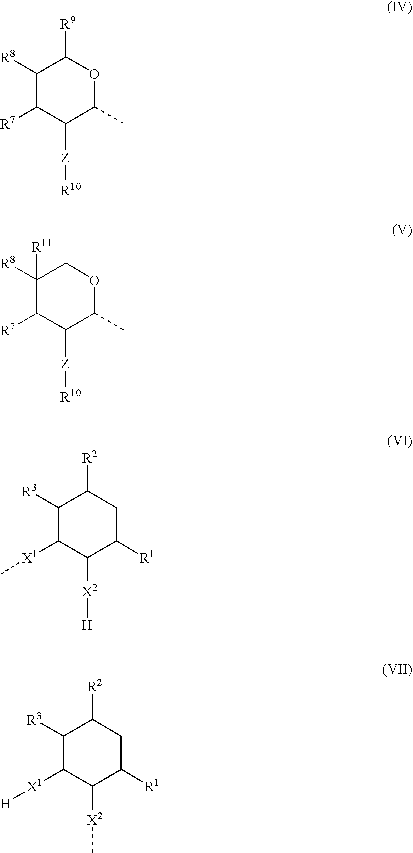
Z can be O, S or NH,
R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1) 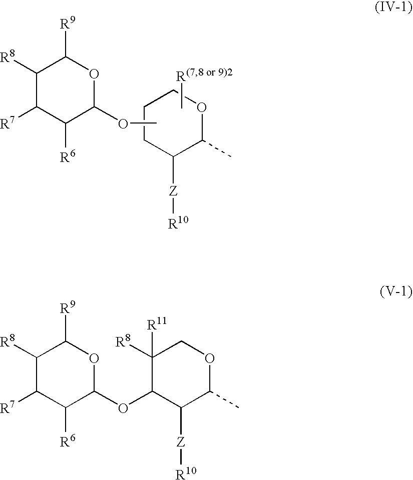
R¹⁰ can be hydrogen, an alkyl group, an amine protecting group, modified amino, hydroxyl protecting or modified hydroxyl group, and
R¹¹ can be a hydrogen, halogen or alkyl group.

3. Compounds having the structure of (Ia), (Ib), (Ic), (Id), (IIa), (IIIa), (IVa) or (Va): 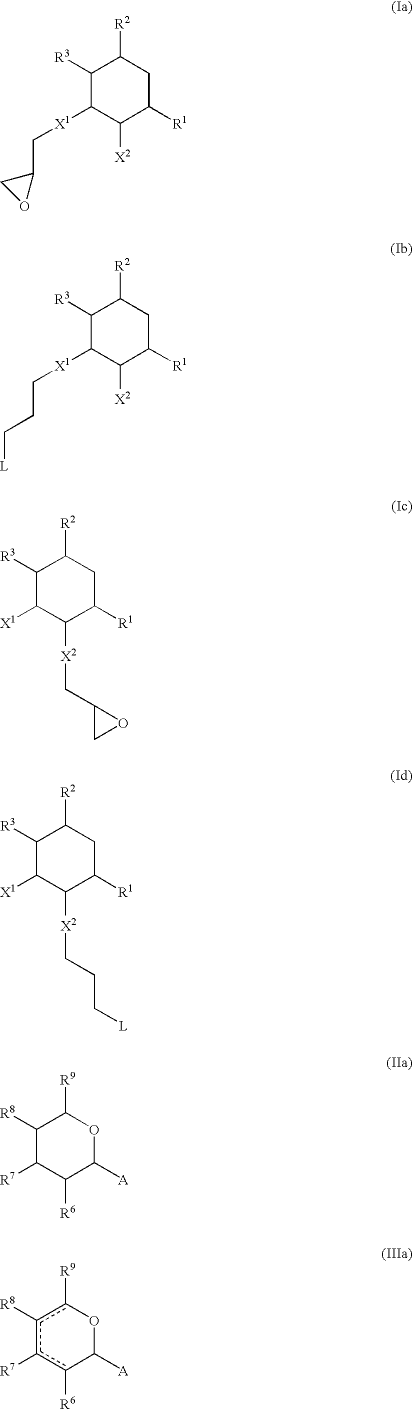 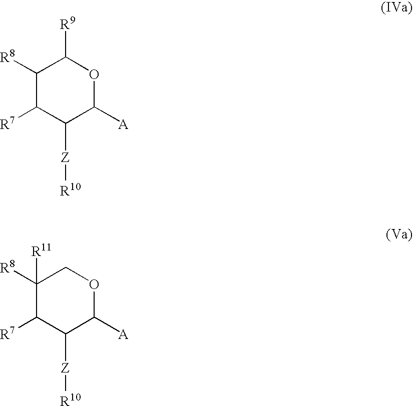
wherein, L is a leaving group,
A is a carbohydrate-activating group,
R¹ and R² are independently amino, protected amino or modified amino,
R³ is selected from the group consisting of the formula (II) or (III),
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen.
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (IIa-1) and (IIIa-1). 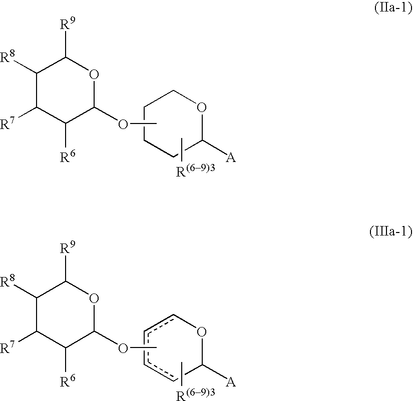
R⁷, R⁸ and R⁹ can also be independently another mono- or disaccharide (II), including disaccharides (IVa-1) and (Va-1) 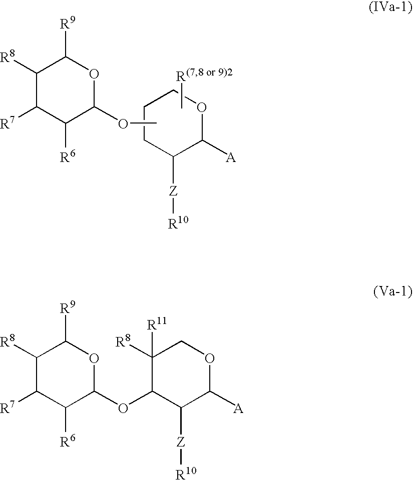
One of X¹ or X² is O, the other can be a protected hydroxyl or modified hydroxyl,
Z can be O, S or NH,
R¹⁰ can be hydrogen, an alkyl group, an amine protecting group, modified amino, hydroxyl protecting or modified hydroxyl group, and
R¹¹ can be a hydrogen, halogen or alkyl group.

4. A method for synthesizing compounds having the structure of (Ia), (Ib), (Ic), (Id), (IIa), (IIIa), (IVa) or (Va): 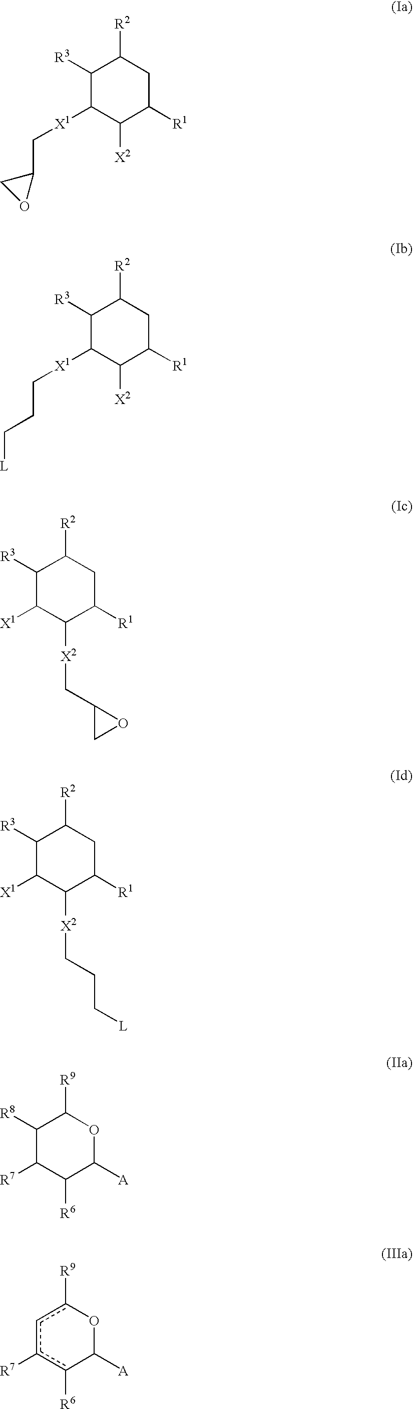 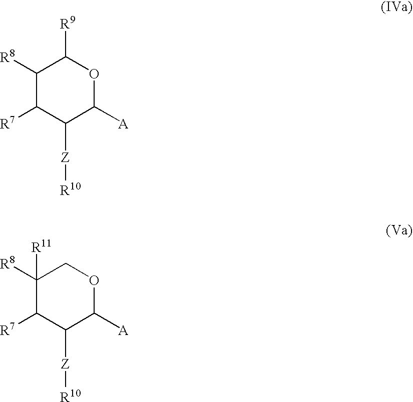
wherein, L is a leaving group,
A is a carbohydrate-activating group,
R¹ and R² are independently amino, protected amino or modified amino,
R³ is selected from the group consisting of the formula (II) or (III),
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen,
R⁶, R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (IIa-1) and (IIIa-1). 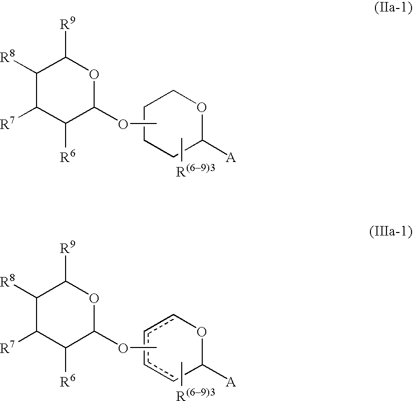
R⁷, R⁸ and R⁹ can also be independently another mono- or disaccharide (II), including disaccharides (IVa-1) and (Va-1) 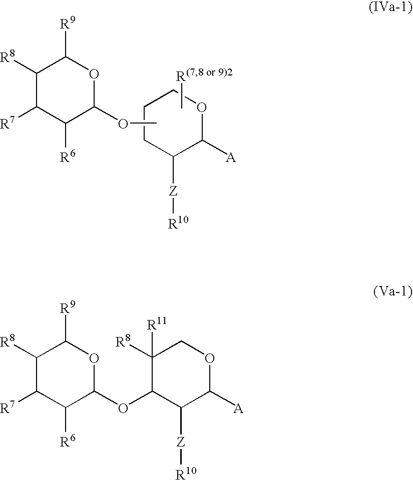
One of X¹ or X² is O, the other can be a protected hydroxyl or modified hydroxyl,
Z can be O, S or NH,
R¹⁰ can be hydrogen, an alkyl group, an amine protecting group, modified amino, hydroxyl protecting or modified hydroxyl group, and
R¹¹ can be a hydrogen, halogen or alkyl group.

5. A pharmaceutical composition for the prophylaxis, amelioration or treatment of a bacterial infection, viral infection, a cancer, or a genetic disorder in mammals avian, fish and reptile species as well as in cell culture, which comprises a therapeutically effective amount of a compound of formula I or a pharmaceutically acceptable salt, prodrug or solvate thereof, 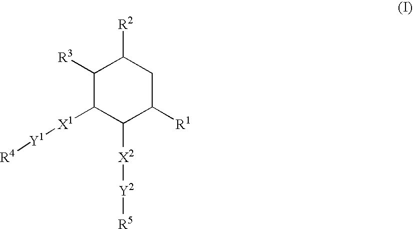
wherein,
R¹ and R² are independently amino, protected amino or modified amino,
X¹ and X² are independently O, S or NH,
Y¹ or Y² is a bond or a divalent linking group,
R³ is selected from the group consisting of the formula (II) or (III): 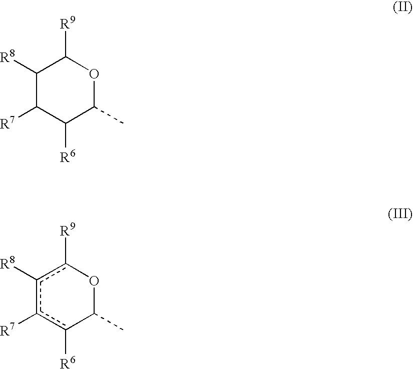
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1). 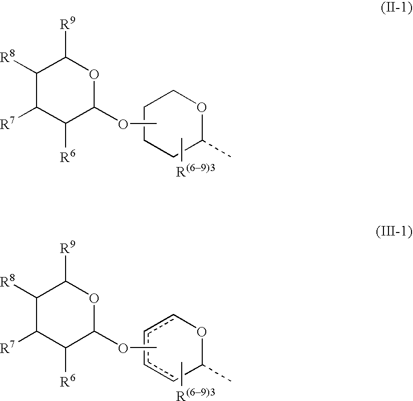
one of R⁴ and R⁵ is hydrogen, hydroxyl protecting or modified hydroxyl group when one of Y¹ or Y² is a bond and the other is selected from a group consisting of formula (II), (III), (IV), (V) or (VI): 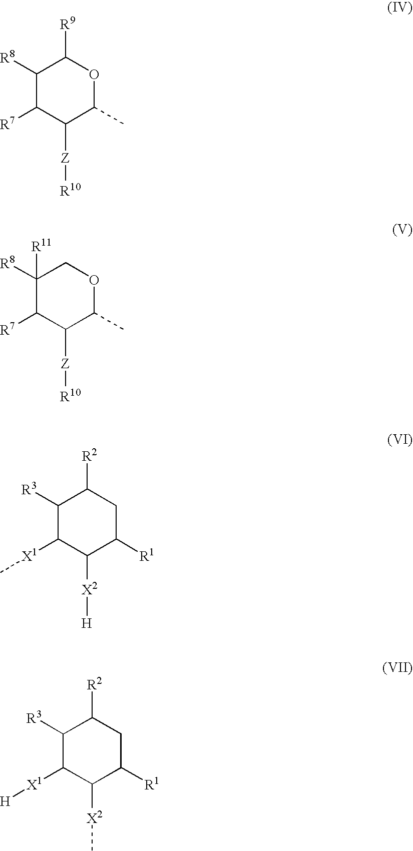
Z can be O, S or NH,
R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1) 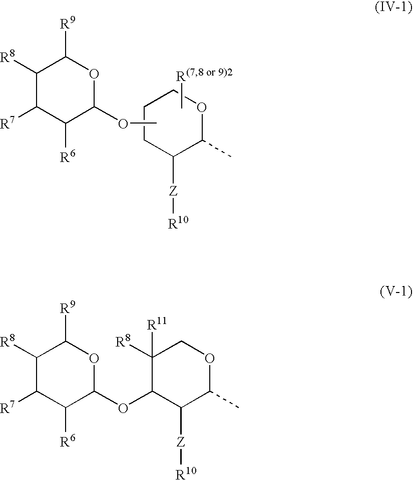
R¹⁰ can be hydrogen, an alkyl group, an amine protecting group, modified amino, hydroxyl protecting or modified hydroxyl group,
R¹¹ can be a hydrogen, halogen or alkyl group,
and a pharmaceutically acceptable carrier.

6. A method for treating, preventing, or ameliorating a bacterial infection, a viral infection, a cancer, or a genetic disorder in mammals avian, fish and reptile species as well as in cell culture, which comprises administering a therapeutically effective amount of a compound of formula I or a pharmaceutically acceptable salt, prodrug or solvate thereof, 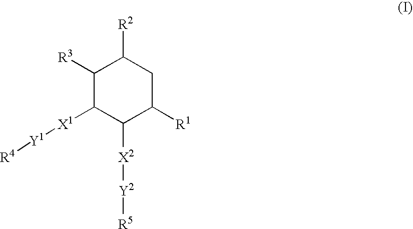
wherein,
R¹ and R² are independently amino, protected amino or modified amino,
X¹ and X² are independently O, S or NH,
Y¹ or Y² is a bond or a divalent linking group,
R³ is selected from the group consisting of the formula (II) or (III): 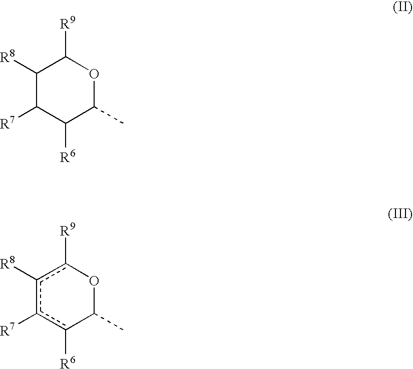
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1). 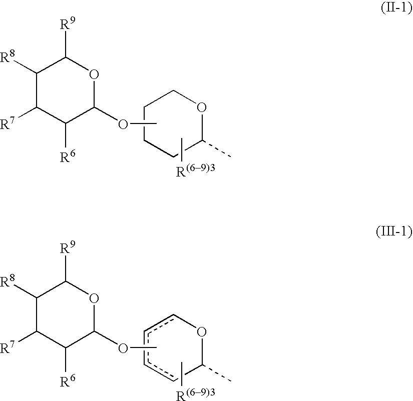
one of R⁴ and R⁵ is hydrogen, hydroxyl protecting or modified hydroxyl group when one of Y¹ or Y² is a bond and the other is selected from a group consisting of formula (II), (III), (IV), (V) or (VI): 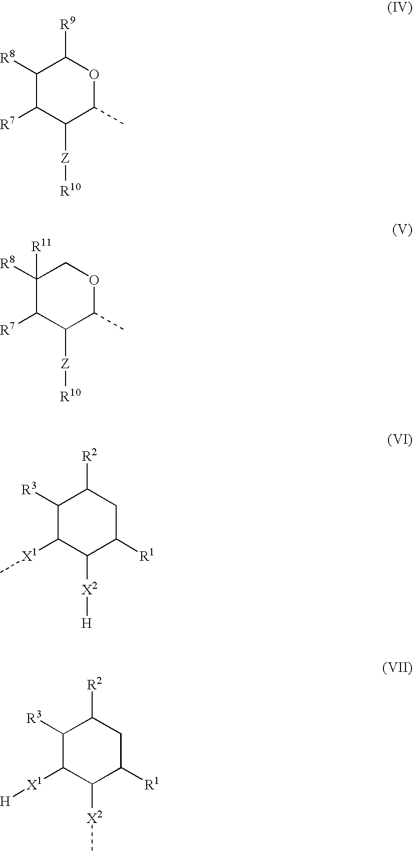
Z can be O, S or NH,
R⁷, R⁸ and R⁹ can also be independently a hydrogen, hydroxyl, protected hydroxyl, modified hydroxyl, amino, protected amino, modified amino, 5 hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1) 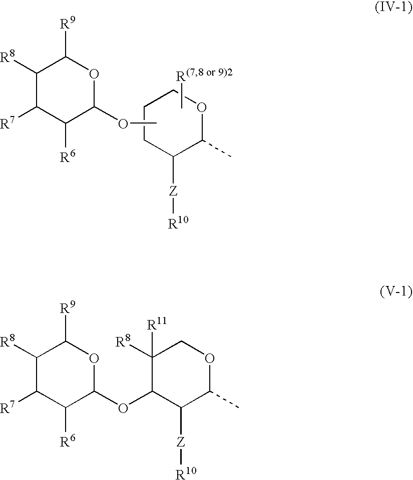
R¹⁰ can be hydrogen, an alkyl group, an amine protecting group, modified amino, hydroxyl protecting or modified hydroxyl group,
R¹¹ can be a hydrogen, halogen or alkyl group,
and a pharmaceutically acceptable carrier.

7. An antibacterial, antiviral or antifungal agent comprising a compound of formula I, 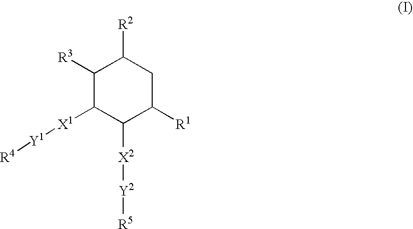
wherein,
R¹ and R² are independently amino, protected amino or modified amino,
X¹ and X² are independently O, S or NH,
Y¹ or Y² is a bond or a divalent linking group,
R³ is selected from the group consisting of the formula (II) or (III): 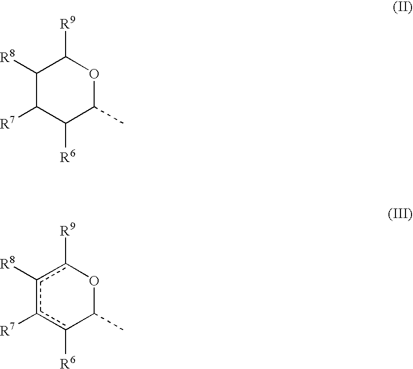
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1). 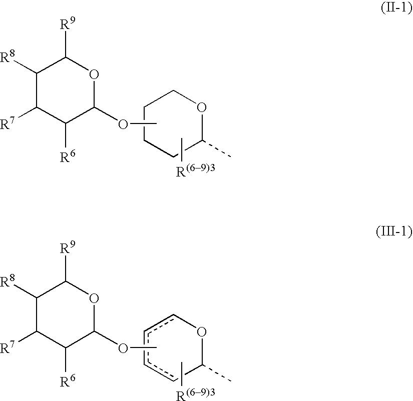
one of R⁴ and R⁵ is hydrogen, hydroxyl protecting or modified hydroxyl group when one of Y¹ or Y² is a bond and the other is selected from a group consisting of formula (II), (III), (IV), (V) or (VI): 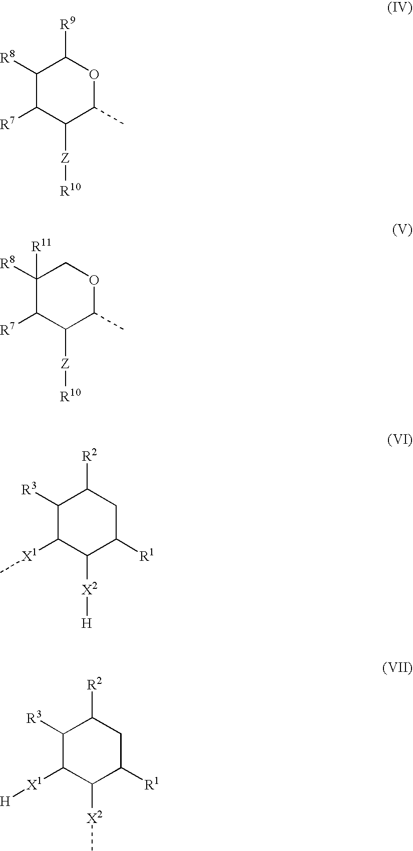
Z can be O, S or NH,
R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1) 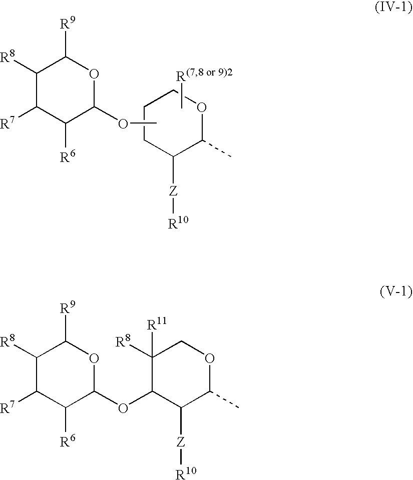
R¹⁰ can be hydrogen, an alkyl group, an amine protecting group, modified amino, hydroxyl protecting or modified hydroxyl group,
R¹¹ can be a hydrogen, halogen or alkyl group,
and an acceptable carrier.

8. A method for preventing, inhibiting, or stopping the growth of bacteria on a surface or within the material of the surface or within the material of the surface, comprising applying to a surface or within the material of the surface an effective amount of an antibacterial agent comprising a compound of formula 1, and an acceptable carrier. 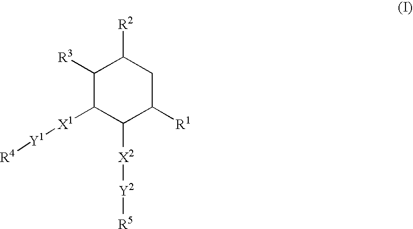
wherein,
R¹ and R² are independently amino, protected amino or modified amino,
X¹ and X² are independently O, S or NH,
Y¹ or Y² is a bond or a divalent linking group,
R³ is selected from the group consisting of the formula (II) or (III): 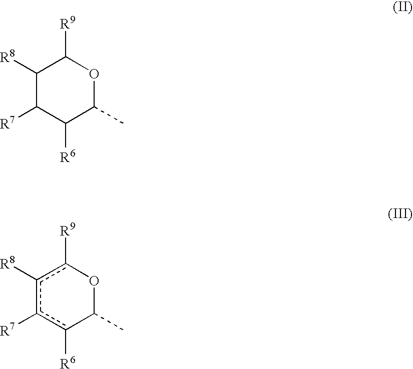
R⁶, R⁷, R⁸ and R⁹ can be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁶, R⁷, R⁸ and R⁹ can be independently another mono- or disaccharide (II), including disaccharides (II-1) and (III-1). 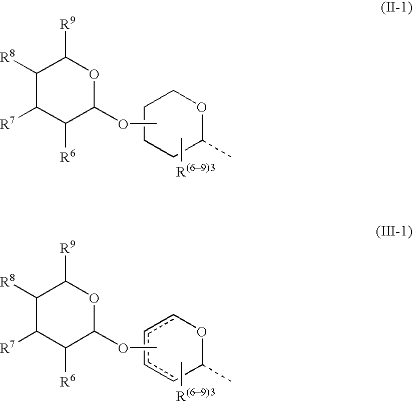
one of R⁴ and R⁵ is hydrogen, hydroxyl protecting or modified hydroxyl group when one of Y¹ or Y² is a bond and the other is selected from a group consisting of formula (II), (III), (IV), (V) or (VI): 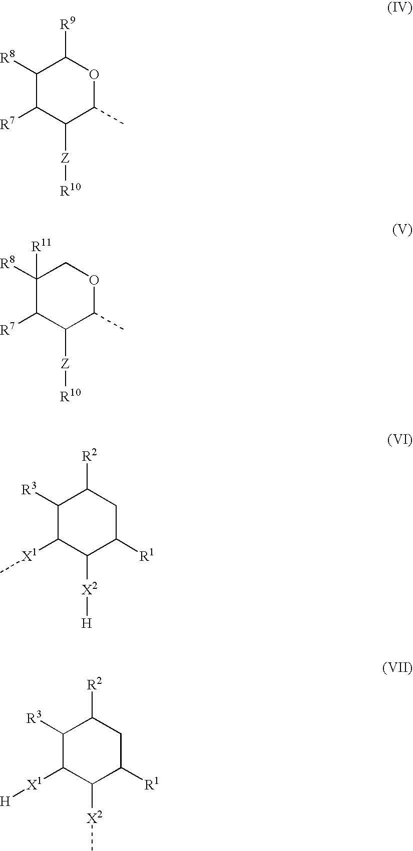
Z can be O, S or NH,
R⁷, R⁸ and R⁹ can also be independently a hydrogen, hydroxyl, protected hydroxyl, modified hydroxyl, amino, protected amino, modified amino, hydroxymethyl, protected hydroxymethyl, aminomethyl, protected aminomethyl, keto or a halogen or R⁷, R⁸ and R⁹ can be independently another mono- or disaccharide (II), including disaccharides (IV-1) and (V-1) 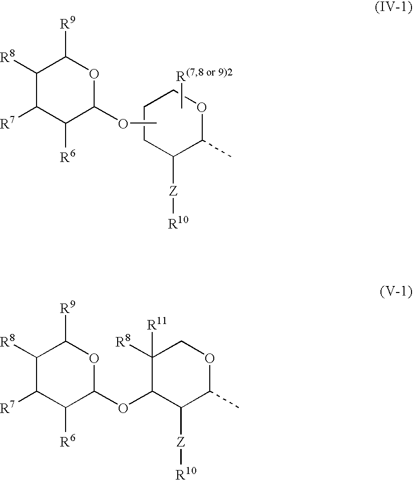
R¹⁰ can be hydrogen, an alkyl group, an amine protecting group, modified amino, hydroxyl protecting or modified hydroxyl group, and
R¹¹ can be a hydrogen, halogen or alkyl group.
